(19) United States
(12) Patent Application Publication
(10) Pub. No.: US 2014/0038181 A1
(43) Pub. Date: Feb. 6, 2014

(54) CHEMICALLY SUBSTITUTED THERMOSENSITIVE PROBES AND COFACTORS FOR HOT START LIGATION

(71) Applicant: TRILINK BIOTECHNOLOGIES, San Diego, CA (US)

(72) Inventors: Alexandre LEBEDEV, San Diego, CA (US); Inna KOUKHAREVA, San Diego, CA (US)

(21) Appl. No.: 13/934,729

(22) Filed: Jul. 3, 2013

Related U.S. Application Data
(63) Continuation of application No. PCT/US2012/020109, filed on Jan. 3, 2012.
(60) Provisional application No. 61/430,133, filed on Jan. 5, 2011.

(51) Int. Cl. C12Q 1/68

(52) U.S. Cl. CPC C12Q 1/6862
USPC 435/6.11; 435/91.2

(57) ABSTRACT

Provided herein are methods for ligase mediated nucleic acid replication and amplification of oligo- and probes containing substituted ligase components, particularly substituted ligase cofactors, substituted oligo- and probe acceptors, substituted oligo- and probe donors, substituted adenylated oligo- and polynucleotide donor intermediates carrying thermolabile group or groups. The substituted ligase components are not active until Hot Start activation step converts them into unsubstituted or natural ligase components, which fully support ligase reaction. The described methods are readily applied to ligation-based assays, especially utilizing Ligase Chain Reaction (LCR), for detection of a nucleic acid sequence where the use of the substituted ligase components improves an overall efficiency of LCR, increase discrimination between matched and mismatched templates and reduces or eliminates appearance of false positive signal. Furthermore, the use of the substituted ligase components reduces or eliminates the false positive signal originated from the template independent and blunt-ended ligation.

This patent application is a continuation of International Application No. PCT/US2012/020109, entitled “Chemically Substituted Thermosensitive Probes and Cofactors for Hot Start Ligation,” filed Jan. 3, 2012, and additionally claims priority to U.S. Provisional Patent Application No. 61/430,133, entitled “Chemically Substituted Thermosensitive Probes and Cofactors for Hot Start Ligation,” filed Jan. 5, 2011, both of which are hereby incorporated by reference in their entirety for all purposes.

### STATEMENT OF GOVERNMENT SUPPORT

The United States Government has certain rights in this invention pursuant to Grant No. GM093562 awarded by the National Institute for General Medical Science.

### FIELD OF THE INVENTION

Provided herein are methods and compositions for ligase mediated nucleic acid replication. In certain particular aspects and embodiments, the methods and compositions are for hot start ligase reaction (HS LR) and hot start ligase chain reaction (HS LCR).

### BACKGROUND OF THE INVENTION

The following description is provided to assist the understanding of the reader. None of the information provided or references cited is admitted to be prior art in the present invention.

Zon et al, US Patent Pub. No. 20070281308 titled Chemically Modified Oligonucleotide Primers For Nucleic Acid Amplification, discloses the use of thermolabile substituted oligonucleotides. Lebedev et al., US Patent Pub. No. 20100003724 titled Chemically Modified Nucleoside 5′-Triphosphates For Thermally Initiated Amplification Of Nucleic Acid, discloses the use of thermolabile substituted nucleoside 5′-triphosphates.

### SUMMARY OF THE INVENTION

Provided herein are methods and compositions for nucleic acid ligation and/or replication. These methods involve the use of nucleic acid ligase, nucleic acid template, ligase cofactor, donor and acceptor probes, and adenylated donor intermediates in template-dependent ligation reactions. In certain aspects, the methods are accomplished by use of a substituted donor probe (SDP) and a substituted acceptor probe (SAP) (collectively referred to herein as “substituted oligonucleotide probes” (SOPs)), substituted adenylated donor intermediate (SADI), and a substituted cofactor (SC), or combinations of any two or more thereof, collectively referred to herein as “substituted ligase components” (SLCs), which provide improved specificity and fidelity in nucleic acid ligation and ligase mediated amplification.

According to one aspect, there are provided methods of ligase mediated nucleic acid replication. In certain embodiments of the aspects provided herein, the method includes replicating nucleic acid using at least one SLC that includes a thermally labile substitution group, where the SLC is one or more ligase components selected from the group consisting of a ligase cofactor, an adenylate-donor intermediate, a donor probe and an acceptor probe.

In a second aspect, there are provided methods for detecting the identity of a nucleic acid residue in a specified position of a target nucleic acid. In certain embodiments of the aspects provided herein, the method includes incubating the target nucleic acid in a reaction mixture including a nucleic acid ligase and an acceptor probe; and (a) a ligase cofactor, and a donor probe, or (b) an adenylated donor intermediate; where at least one of the ligase cofactor, donor probe, adenylated donor intermediate or acceptor probe is a SLC having a thermally labile substitution group; and monitoring ligation, where the amount of ligation is indicative of the presence or absence of the specified position of a target nucleic acid.

In a third aspect, there are provided methods for detecting the presence or absence of one of the alternative bases at a single nucleotide polymorphism (SNP) site in a target nucleic acid. In certain embodiments of the aspects provided herein, the method includes incubating the target nucleic acid in a reaction mixture including a nucleic acid ligase and an acceptor probe; and (a) a ligase cofactor, and a donor probe or (b) an adenylated donor intermediate, where at least one of the ligase cofactor, donor probe, adenylated donor intermediate or acceptor probe is a SLC having a thermally labile substitution group; and monitoring ligation, where the amount of ligation indicates the presence or absence of the one of the alternative bases at the SNP in the target nucleic acid.

In a fourth aspect, there are provided methods for distinguishing the presence of a first nucleic acid sequence or a second nucleic acid sequence in a target nucleic acid. In certain embodiments of the aspects provided herein, the methods include incubating the target nucleic acid in a reaction mixture including a nucleic acid ligase and an acceptor probe; and (a) a ligase cofactor, and a donor probe or (b) an adenylated donor intermediate, where at least one of the ligase cofactor, donor probe, adenylated donor intermediate or acceptor probe is a SLC having a thermally labile substitution group; and monitoring ligation, where the presence or amount of ligated nucleic acid is indicative of the presence or amount of the first nucleic acid sequence in the target nucleic acid and/or the absence of the second nucleic acid sequence in the target nucleic acid and the absence of ligation is indicative of the absence of the first nucleic acid sequence in the target nucleic acid.

In a fifth aspect, there are provided methods determining the presence or absence of a particular nucleotide at a specified position of a target nucleic acid. In certain embodiments of the aspects provided herein, the methods include incubating the target nucleic acid in a reaction mixture including a nucleic acid ligase and an acceptor probe; and (a) a ligase cofactor, and a donor probe or (b) an adenylated donor intermediate, where at least one of the ligase cofactor, donor probe, adenylated donor intermediate or acceptor probe is a SLC having a thermally labile substitution group; and monitoring ligation, where the presence of ligated nucleic acid is indicative of the presence of the particular nucleotide at the specified position of the target nucleic acid and the absence of ligation is indicative of the absence of the particular nucleotide at the specified position of the target nucleic acid.

In a sixth aspect, there are provided kits that include the compositions provided herein and kits for performing the methods provided herein. Kits that include SLCs for performing ligation as described herein are also provided. For example, kits may contain ligase enzyme and SCs to detect common nucleic acid targets such as allele-specific products. The kit containing a SLC may include a container marked for nucleic acid ligation, instructions for performing nucleic acid ligation and/or one or more reagents selected from the group consisting of SC, nucleic acid ligase, and reaction buffer. The kit containing a SLC may also include one or more donor and acceptor probes and/or an adenylate-donor intermediate. In one embodiment, the donor probe, acceptor probe and/or adenylate-donor intermediate are substituted. The kits may include a container marked for nucleic acid ligation, instructions for performing nucleic acid ligation and at least one SLC and/or one or more reagents selected from the group consisting of ligase cofactor, nucleic acid ligase, magnesium, donor probes, acceptor probes, and reaction buffer.

In a seventh aspect, there are provided methods for identifying SLCs for performing ligation. In some embodiments, the methods identify a substituted component that has increased specificity relative to the natural ligation component, unsubstituted ligation component or equivalent thereof, or other SLC. For example, the methods may evaluate the performance of a substituted component in the presence of a matched or mismatched template. In some embodiments, the mismatched region will hybridize to the donor probe, and in other embodiments, the mismatched region will hybridize to the acceptor probe. In some embodiments the performance of a substituted component will be evaluated for reduction or inhibition of ligation activity in the absence of a nucleic acid template. In some embodiments, the methods identify a SLC that has improved ligation specificity relative to the natural or unsubstituted component. In some embodiments, the methods allow identification of a SLC that has a similar efficiency of ligation relative to the natural or unsubstituted component for matched nucleic acid. In other embodiments, the methods allow identification of a SLC that has improved ligation specificity in the presence of matched and mismatched nucleic acid templates relative to the natural or unsubstituted component. In yet other embodiments, the methods evaluate a SLC for ligation amount or yield where there are one or more base-pair mismatches at the ligation junction or within 10, or within 9, or within 8, or within 7, or within 6, or within 5, or within 4, or within 3, or within 2 or within 1 base(s) of the ligation junction.

In an eighth aspect, there are provided methods for detecting the identity of a nucleic acid residue in a specified position of a target nucleic acid. In certain embodiments of the aspects provided herein, the method includes incubating the target nucleic acid in a reaction mixture including a cofactor dependent nucleic acid ligase, a ligase cofactor, a donor probe and an acceptor probe, where at least one of the ligase cofactor, donor probe or acceptor probe is a SLC having a thermally labile substitution group; and monitoring ligation of the donor and acceptor probes, where the amount of ligation is indicative of the presence or absence of the specified position of a target nucleic acid. In certain embodiments, the method includes an adenylate-donor intermediate having a thermally labile substitution group.

In a ninth aspect, there are provided methods for detecting the identity of a nucleic acid residue in a specified position of a target nucleic acid. In certain embodiments of the aspects provided herein, the method includes incubating the target nucleic acid in a reaction mixture including a nucleic acid ligase, an adenylated donor intermediate and an acceptor probe, where at least one of the adenylated donor intermediate and acceptor probe is a SLC having a thermally labile substitution group; and monitoring ligation of the adenylated donor intermediate and acceptor probe, where the amount of ligation is indicative of the presence or absence of the specified position of a target nucleic acid.

In a tenth aspect, there are provided methods for detecting the presence or absence of one of the alternative bases at a SNP site in a target nucleic acid. In certain embodiments of the aspects provided herein, the method includes incubating the target nucleic acid in a reaction mixture including a cofactor dependent nucleic acid ligase, a substituted ligase cofactor, a donor probe and an acceptor probe, where at least one of the ligase cofactor, donor probe or acceptor probe is a SLC having a thermally labile substitution group; and monitoring ligation of the donor and acceptor probes, where the amount of ligation indicates the presence or absence of the one of the alternative bases at the SNP in the target nucleic acid. In certain embodiments, the method includes an adenylate-donor intermediate having a thermally labile substitution group.

In an eleventh aspect, there are provided methods for detecting the presence or absence of one of the alternative bases at a SNP site in a target nucleic acid. In certain embodiments of the aspects provided herein, the method includes incubating the target nucleic acid in a reaction mixture including a nucleic acid ligase, an adenylated donor intermediate and an acceptor probe, where at least one of the adenylated donor intermediate and acceptor probe is a SLC having a thermally labile substitution group; and monitoring ligation of the adenylated donor intermediate and acceptor probe, where the amount of ligation indicates the presence or absence of the one of the alternative bases at the SNP in the target nucleic acid.

In a twelfth aspect, there are provided methods for distinguishing the presence of a first nucleic acid sequence or a second nucleic acid sequence in a target nucleic acid. In certain embodiments of the aspects provided herein, the methods include incubating the target nucleic acid in a reaction mixture including a cofactor dependent nucleic acid ligase, a ligase cofactor, a donor probe and an acceptor probe, where at least one of the ligase cofactor, donor probe or acceptor probe is a SLC having a thermally labile substitution group; and monitoring ligation of the donor and acceptor probes, where the presence or amount of ligated nucleic acid is indicative of the presence or amount of the first nucleic acid sequence in the target nucleic acid and/or the absence of the second nucleic acid sequence in the target nucleic acid and the absence of ligation is indicative of the absence of the first nucleic acid sequence in the target nucleic acid. In certain embodiments, the method includes an adenylate-donor intermediate having a thermally labile substitution group.

In a thirteenth aspect, there are provided methods for distinguishing the presence of a first nucleic acid sequence or a second nucleic acid sequence in a target nucleic acid. In certain embodiments of the aspects provided herein, the method includes incubating the target nucleic acid in a reaction mixture including a nucleic acid ligase, an adenylated donor intermediate and an acceptor probe, where at least one of the adenylated donor intermediate and acceptor probe is a SLC having a thermally labile substitution group; and monitoring ligation of the adenylated donor intermediate and acceptor probe, where the presence or amount of ligated nucleic acid is indicative of the presence or amount of the first nucleic acid sequence in the target nucleic acid and/or the absence of the second nucleic acid sequence in the target nucleic acid and the absence of ligation is indicative of the absence of the first nucleic acid sequence in the target nucleic acid.

In a fourteenth aspect, there are provided methods determining the presence or absence of a particular nucleotide at a specified position of a target nucleic acid. In certain embodiments of the aspects provided herein, the methods include incubating the target nucleic acid in a reaction mixture including a cofactor dependent nucleic acid ligase, a ligase cofactor, a donor probe and an acceptor probe, where at least one of the ligase cofactor, donor probe or acceptor probe is a SLC having a thermally labile substitution group; and monitoring ligation of the donor and acceptor probes, where the presence of ligated nucleic acid is indicative of the presence of the particular nucleotide at the specified position of the target nucleic acid and the absence of ligation is indicative of the absence of the particular nucleotide at the specified position of the target nucleic acid. In certain embodiments, the method includes an adenylate-donor intermediate having a thermally labile substitution group.

In a fifteenth aspect, there are provided methods determining the presence or absence of a particular nucleotide at a specified position of a target nucleic acid. In certain embodiments of the aspects provided herein, the method includes incubating the target nucleic acid in a reaction mixture including a nucleic acid ligase, an adenylated donor intermediate and an acceptor probe, where at least one of the adenylated donor intermediate and acceptor probe is a SLC having a thermally labile substitution group; and monitoring ligation of the adenylated donor intermediate and acceptor probe, where the presence of ligated nucleic acid is indicative of the presence of the particular nucleotide at the specified position of the target nucleic acid and the absence of ligation is indicative of the absence of the particular nucleotide at the specified position of the target nucleic acid.

In conjunction with any of the various aspects, embodiments, compositions and methods disclosed herein, provided are SLCs, particularly SC, SADI, SAP, SDP, and combinations of any two or more thereof. In particular embodiments, the SLCs include those as depicted in Formulas I-V described in further detail herein.

The SLCs of the methods and compositions provided herein have significant advantages. For example, an end user can use the same ligation and amplification protocols and methods already in use with unsubstituted/natural ligation components, i.e., donor and acceptor probes and ligase cofactors such as ATP and NAD+. The SLCs of the methods and compositions provided herein are compatible with existing ligation systems and reagents; no additional enzymes or reagents are needed but can be used.

Ligation performed with Hot Start activation using SLCs provided herein preferably results in at least about the same efficacy for nucleic acid ligation in the presence of complementary target as compared to the unsubstituted ligation component. In preferred embodiments, without Hot Start activation the SLCs do not support ligase reaction. Preferably, ligation is considered impaired when a SLC is at least 50% less efficacious as a reagent in a ligation reaction compared to its corresponding unsubstituted ligation component, preferably at least 60% less efficacious, preferably at least 70% less efficacious, more preferably at least 80% less efficacious, more preferably at least 90% less efficacious, more preferably at least 95% less efficacious, more preferably at least 99% less efficacious and most preferably 100% less efficacious as a SC in a ligation reaction than its corresponding unsubstituted ligation component. One of ordinary skill in the art is able to readily determine the level of ligation activity and efficacy of SLC.

The SLCs of the methods and compositions provided herein preferably have no or reduced efficacy for nucleic acid ligation in without Hot Start activation as compared to the unsubstituted ligation component.

The methods and compositions herein provide improved methods and compositions for nucleic acid ligation, nucleic acid replication and amplification (such as LCR), in general. In particular embodiments, the methods and compositions are directed to the use of SLCs in enzymatic ligation reactions. In certain embodiments, the process of nucleic acid ligation employs one or more SC, SDP, SAP, and/or SADI the presence of which impedes the formation of undesired ligation products in the presence of mismatched nucleic acid template or in the absence of template at all.

SCs with Thermally Labile Substitution Groups

In conjunction with any of the aspects, embodiments, compositions and methods disclosed herein, a SC includes a thermolabile substitution group that is sensitive to the temperature of the reaction mixture and can dissociate or cleave at an elevated temperature resulting in the corresponding natural or unsubstituted ligase cofactor. In certain embodiments, the SC is a substituted ATP, NAD+ or GTP.

In conjunction with any of the various aspects, embodiments, compositions and methods disclosed herein, SCs include derivatives of ATP and/or NAD+ having a thermolabile group at the sugar, base and/or phosphate moiety of the molecules. In certain embodiments, substituted ATP or NAD+ includes a thermolabile substitution at any or all hydroxyl groups of the ribose moiety or moieties, at the 6-amino group of adenine base and/or at the gamma-phosphate of the triphosphate chain of ATP or pyrophosphate chain of NAD+, respectively.

In conjunction with any of the aspects, embodiments, compositions and methods disclosed herein, substituted ATP and/or NAD+ include a thermolabile substitution at a sugar moiety as shown, for example, in Formulas I and II, respectively.

In conjunction with any of the aspects, embodiments, compositions and methods disclosed herein, substituted ATP has a bis-2′,3′-substitution, i.e., groups other than a hydroxyl group at the 2′ and 3′-positions. In some embodiments, substituted ATP has a single 2′- or 3′-substitution, i.e., one group other than a hydroxyl group at either 2′- or 3′-positions (Formula I).

In some embodiments, substituted NAD+ has a bis-2′,3′-substitution at the adenosine moiety, i.e., groups other than a hydroxyl group at the 2′ and 3′-positions of the adenosine moiety of NAD+ molecule. In some embodiment, substituted NAD+ has a single 2′- or 3′-substitution at the adenosine moiety, i.e., a group other than a hydroxyl group at the either 2′- or 3′-positions of the adenosine moiety of the NAD+ molecule. Positions 2′- and 3′-correlate to positions X⁵ and X⁶ of Formula II respectively.

In some embodiments, substituted NAD+ has a bis-2″,3″-substitution at nicotine amide riboside moiety, i.e., groups other than a hydroxyl group at the 2″,3″-positions of the nicotine amide riboside moiety of NAD+ molecule. In preferred embodiments, the substituted NAD+ has a single 2″- or 3″-substitution at the nicotine amide riboside moiety, i.e., a group other than a hydroxyl group at either 2″- or 3″-positions of the nicotine amide riboside moiety of NAD+ molecule. Positions 2″- and 3″-correlate to positions X^{5a} and X^{6a} of Formula II respectively.

In some embodiments, substituted NAD+ has either a single or double substitution at each sugar moiety of the NAD+ molecule, i.e., a group other than a hydroxyl group at either 2′,3′,2″ or 3″ positions of each ribose moiety of the NAD+ molecule (Formula II).

In some embodiments, substituted ATP and NAD+ include a thermolabile substitution group at the adenine base as shown, for example, in Formulas I and II, respectively.

In some embodiments, substituted ATP includes a substitution group attached to the 6-amino group of the adenine base. In some embodiments, the substitution group at the 6-amino group of adenine base dissociates, cleaves or converts to a 6-NH₂ group during the initial denaturation step of a replication reaction. One of skill in the art would be able to determine the parameters in which the initial denaturation step occurs based on the application being performed with the 6-amino substituted ATP provided herein.

In some embodiments, substituted NAD+ includes a substitution at the 6-amino group of the adenine base. In some embodiments, the substitution group at the 6-amino group of the adenine base dissociates, cleaves or converts to a 6-NH₂ group during the initial denaturation step of a replication reaction. One of skill in the art would be able to determine the parameters in which the initial denaturation step occurs based on the application being performed with the 6-amino substituted NAD+ provided herein.

In conjunction with any of the aspects, embodiments, compositions and methods disclosed herein, a SC includes a substitution at a polyphosphate moiety. In some embodiments a SC is ATP or NAD+ substituted with thermolabile group at any position of a phosphate moiety. In some embodiments, ATP is substituted at the gamma position of a triphosphate moiety. In some embodiments, NAD+ is substituted at position P¹ or P² of a pyrophosphate moiety.

In some embodiments, substituted ATP includes a thermolabile substitution group at the gamma-phosphate of the triphosphate chain. In some embodiments, a substituted gamma-phosphate of the triphosphate chain converts to an unsubstituted gamma-phosphate group during the initial denaturation step of the replication reaction generating unsubstituted ATP. One of skill in the art would be able to determine the parameters in which the initial denaturation step occurs based on the application being performed with the gamma-phosphate substituted ATP (Formula I).

In some embodiments, substituted NAD+ includes one or more thermolabile substitution groups at position P¹ or P² of a pyrophosphate moiety. In some embodiments, the substituted pyrophosphate moiety converts to an unsubstituted pyrophosphate moiety during the initial denaturation step of the replication reaction generating unsubstituted NAD+. One of skill in the art would be able to determine the parameters in which the initial denaturation step occurs based on the application being performed with the pyrophosphate substituted NAD+ (Formula II).

In conjunction with any of the aspects, embodiments, compositions and methods disclosed herein, DNA ligase mediated nucleic acid replication, is HS LCR. In certain embodiments the use of SCs impedes nucleic acid ligase mediated phosphodiester bond formation between adjacent 3′-hydroxyl of an acceptor probe and 5′-phosphoryl termini of a donor probe in the presence of nucleic acid template (e.g., RNA or DNA) prior to the initial heat denaturation step, Hot Start. In certain embodiments, a SC does not support nucleic acid ligase mediated non-templated and blunt-ended ligation between 3′-hydroxyl of the acceptor probe and 5′-phosphoryl termini of the donor probe.

In conjunction with any of the aspects, embodiments, compositions and methods disclosed herein, a SC as disclosed herein may convert to unsubstituted cofactors during and after the initial heat denaturation step, Hot Start, of the ligase mediated nucleic acid replication and, where applicable, during a subsequent thermal cycle sequence such as LCR. In some embodiments a partial or complete conversion of the substitution group of SC occurs during incubation at approximately 95° C. for approximately 0.1-120 minutes. Examples of thermolabile substitution groups for nucleosides and nucleotides of the compositions and methods provided herein are described in Beaucage et. al., U.S. Pat. No. 7,355,037; Chmielewski et. al., J. Org. Chem. 68, 10003-10012 (2003); Koukhareva and Lebedev, Analytical Chemistry 81, 4955-4962 (2009); and Lebedev and Koukhareva, US Patent Application 20100003724.

In conjunction with any of the aspects, embodiments, compositions and methods disclosed herein, substitution at a sugar and/or base and/or phosphate group of a ligase cofactor may prevent formation of an activated ligase-adenylate intermediate in which AMP residue, derived from a SC, linked via a phosphoramide bond to the e-amino group of a lysine residue of the ligase. This type of SC represents an “inactive ligase cofactor” (ILC).

In conjunction with any of the aspects, embodiments, compositions and methods disclosed herein, a sugar and/or base and/or phosphate group SC can transfer its substituted adenylyl residue to the e-amino group of a lysine of the ligase forming a ligase-adenylate intermediate. If this ligase-adenylate intermediate is inactive due to the presence of a substitution group at a sugar and/or adenine base of the adenylate moiety, it impedes further transfer of the adenylate moiety to a 5′-phosphate of the donor probe thus preventing formation of adenylated donor probe intermediate. This type of SC represents an “enzyme inactivating cofactor” (EIC).

In certain embodiments a partial or complete conversion of the activatable cofactor from inactive state to active state occurs during incubation at approximately 95° C. for approximately 0.1-120 minutes. In preferred embodiments, conversion occurs with respect to temperature and does not require enzymes, additional chemicals, or modified reaction conditions other than those normally used in or ligation reactions with natural cofactors.

In conjunction with any of the aspects, embodiments, compositions and methods disclosed herein, substituted ATP or NAD+ includes a substitution at the adenine base, e.g. 7-deazaadenine and 8-aza-7-deazaadenine or guanine can replace the adenine base. Substituted ATP and NAD+ derivatives may include one or more of the chemical structures depicted in Formulas I and II further described herein.

In one aspect, the methods and compositions herein provide for methods of synthesis and preparation of SCs as disclosed herein. In yet another aspect, provides are methods of manufacturing of SCs comprising one or more of thermolabile substitution group and one or more thermostable substitution groups depicted in Formulas I and II further described herein.

In conjunction with any of the aspects, embodiments, compositions and methods disclosed herein, substituted ATP and derivatives thereof include compounds of Formula I:

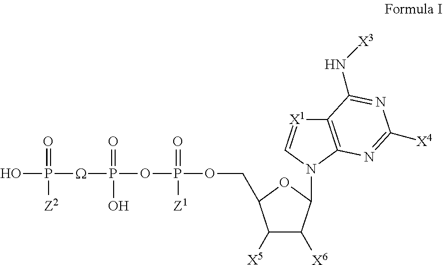

wherein:
X¹ is selected from the group consisting of C—X² and N;
X² is selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
Z¹ is selected from group consisting of OH, OR¹, SH, SR¹, CH₃, CH₂CH₃, Phenyl, BH₃⁻, NH₂, NHR¹, and NR¹R³;
Z² is selected from the group consisting of OH, OR¹, SH, SR¹, NHR¹, NR¹R², F, phosphate, substituted phosphate, substituted polyphosphate, substituted phosphonate, sulfate, sulphonate, O-acyl, S-acyl, NH-acyl, and NR¹-acyl,
   wherein Z² is optionally a thermolabile substitution group;
Ω is selected from the group consisting of O, CR¹R², NR¹, and N—OR³;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl, wherein X³ is optionally a thermolabile substitution group;
X⁴ is selected from the group consisting of hydrogen, NH₂, NHR¹, OH, OR¹, SH, SR¹ and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
X⁵ and X⁶ are each independently selected from the group consisting of hydrogen, OH,

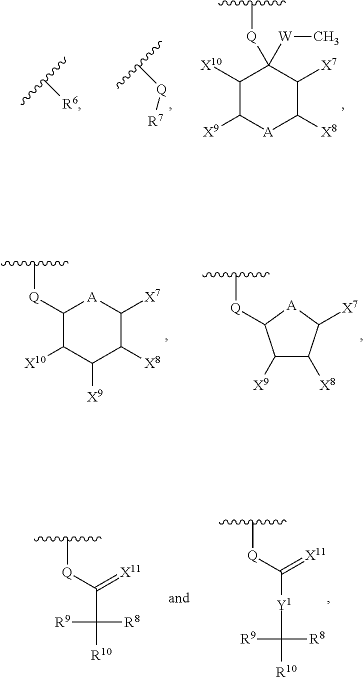

wherein X⁵ and X⁶ are each optionally a thermolabile substitution group;
Q is selected from group consisting of O, S, NH, NR¹, NOR¹, CHR¹, and CR¹R²;
R⁶ is selected from the group consisting of inorganic acid residue, or derivative thereof, with the exception of carbonic acid, where the derivatives may include but are not limited to halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R³, R⁷, R⁸, R⁹ and R¹⁶ is independently selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting O, S, NH, NR¹, NOR¹, CHR¹, and CR¹R²; and
each A, Y¹ and W is independently selected from the group consisting of O, S, NH, NR¹, NOR¹, CHR¹, and CR¹R²;
wherein at least one of Z², X³, X⁵ and X⁶ are each independently a thermolabile substitution group.

Preferred embodiments of substituted ATP and derivatives thereof have the structures of Formulas IA-IF as follows:

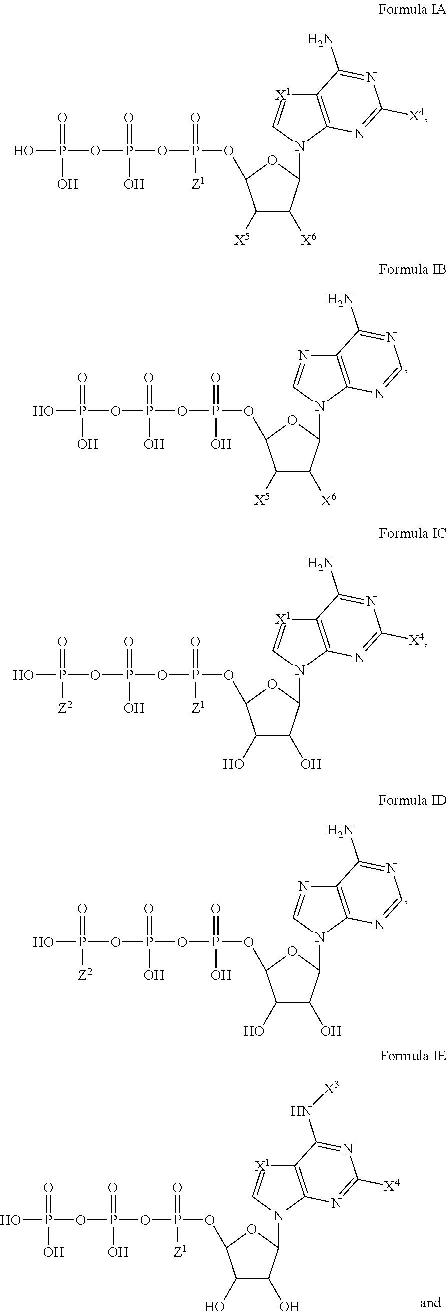

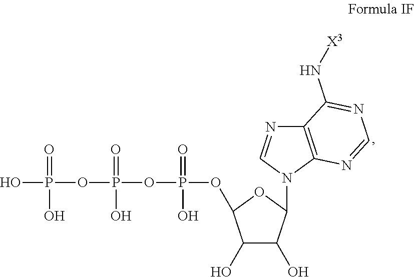

wherein:
Z¹, X¹ and X⁴ are as defined in Formula I; and
at least one of Z², X³, X⁵ and X⁶ is a thermolabile substitution group as defined in Formula I.
   In certain embodiments, both X⁵ and X⁶ are a thermolabile substitution group as defined in Formula I.

Preferred embodiments of substituted ATP and derivatives thereof have the structures of Formulas IBa-IBc as follows:

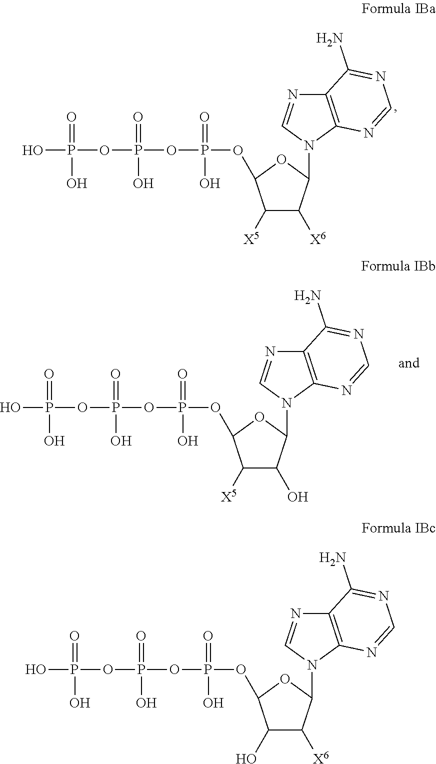

wherein:
at least one of X⁵ and X⁶ is a thermolabile substitution group independently selected from the group consisting of O-[4-methoxy]tetrahydropyranyl; O-tetrahydropyranyl; O-tetrahydrofuranyl; O-phenoxyacetyl; O-methoxyacetyl; O-(p-toluene)sulfonate; O-[4-methoxy]-tetrahydrothiopyranyl; O-tetrahydrothiopyranyl; O-[5-methyl]-tetrahydrofuranyl; O-[2-methyl, 4-methoxy]-tetrahydropyranyl; O-[5-methyl]-tetrahydropyranyl, O-tetrahydrothiofuranyl, O-2-[tert-butoxy]ethyl, O-2-[cyclohexoxy]ethyl, O-2-[isopropoxy]ethyl, O-2-[isobutoxy]ethyl, O-2-[ethoxy]ethyl, O-2-[propoxy]ethyl, O-2-[2-ethylhexoxy]ethyl, O-2-[butoxy]ethyl, O-2-[dodecoxy]ethyl, O-2-methyl-2-[ethoxy]ethyl, and O-2-[tert-pentoxy]ethyl.

Preferred embodiments of substituted ATP and derivatives thereof have the structure of Formula IDa:

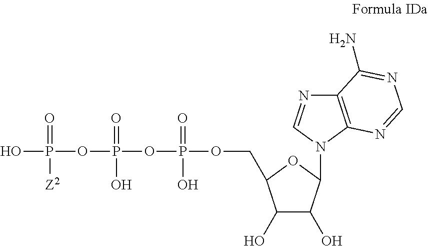

wherein:
Z² is a thermolabile substitution group selected from the group consisting of NH-methyl, NH-ethyl, NH-propyl, NH-butyl, NH-phenyl, NH-p-nitrophenyl, NH-o-nitrophenyl, NH-m-nitrophenyl, NH-[(4-azido-2,3,5,6-tetrafluorobenzoyl)amino]propyl, imidazolyl, triazolyl, O-2-cyanoethyl, O-p-nitrophenyl, O-o-nitrophenyl, O-m-nitrophenyl, S-2-cyanoethyl, S-p-nitrophenyl, S-o-nitrophenyl, S-m-nitrophenyl, O-Acetyl, O-benzoyl, O-2,4,6-trimethylcarbonyl, O-phosphoryl, and O-pyrophosphoryl.

Preferred embodiments of substituted ATP and derivatives thereof have the structure of Formula IFa:

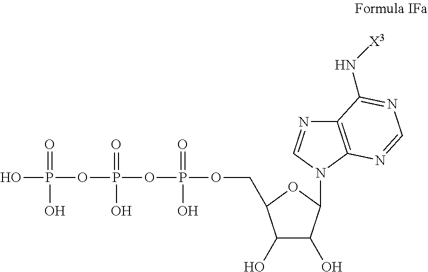

wherein:
X³ is a thermolabile substitution group selected from the group consisting of methoxycarbonyl, ethoxycarbonyl, 9-fluorenylmethoxycarbonyl, 2,2,2-trichloroethoxycarbonyl, 2-trimethylsylylethoxycarbonyl, tert-butoxycarbonyl, allyloxycarbonyl, benzyloxycarbonyl, phenyloxycarbonyl, p-nitrophenyloxycarbonyl, cyclohexyloxycarbonyl, phenoxyacetyl, methoxyacetyl, benzoyl, acetyl, dimethoxytrityl, monomethoxytrityl, trityl, N,N-dimethylaminomethylidene, N,N-diphenylaminomethylidene, and NN-dibenzylaminomethylidene.

In conjunction with any of the aspects, embodiments, compositions and methods disclosed herein, substituted NAD+ and derivatives thereof include compounds of Formula II:

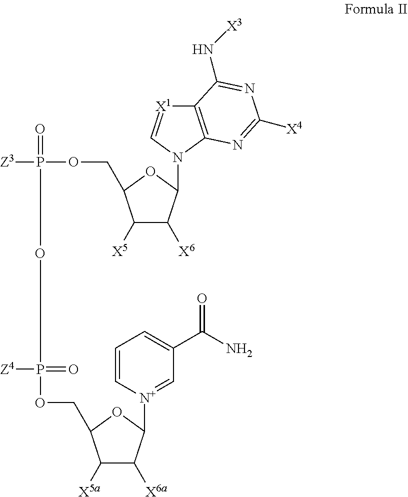

wherein:
X¹ is selected from the group consisting of C—X² and N;
X² is selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
Z³ and Z⁴ are each independently selected from the group consisting of OH, OR¹, SH, SR¹, NHR¹, NR¹R², F, phosphate, substituted phosphate, substituted polyphosphate, substituted phosphonate, sulfate, sulphonate, O-acyl, S-acyl, NH-acyl, NR¹-acyl, CH₃, and BH₃⁻;
   wherein Z³ and Z⁴ are each optionally a thermolabile substitution group;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl, wherein X³ is optionally a thermolabile substitution group;
X⁴ is selected from the group consisting of hydrogen, NH₂, NHR¹, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁵, X⁶, X^{5a} and X^{6a} is independently selected from the group consisting of hydrogen, OH,

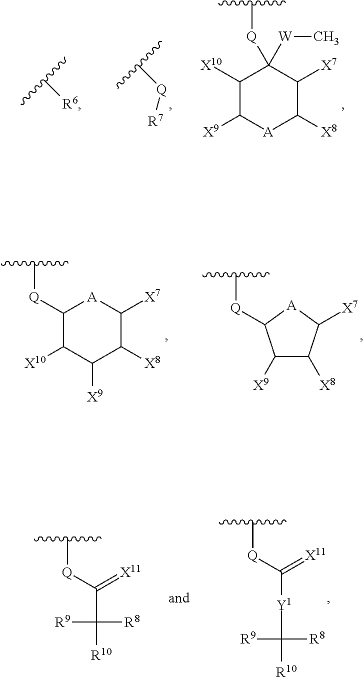

wherein X⁵, X⁶, X^{5a} and X^{6a} are each optionally a thermolabile substitution group;
Q is selected from group consisting of O, S, NH, NR¹, NOR¹, CHR¹, and CR¹R²;
R⁶ is selected from the group consisting of inorganic acid residue, or derivative thereof, with the exception of carbonic acid, where the derivatives may include but are not limited to halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R³, R⁷, R⁸, R⁹ and R¹⁰ is independently selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting O, S, NH, NR¹, NOR¹, CHR¹, and CR¹R²; and
each A, Y¹ and W is independently selected from the group consisting of O, S, NH, NR¹, NOR¹, CHR¹, and CR¹R²;
wherein at least one of Z³, Z⁴, X³, X⁵, X⁶, X^{5a} or X^{6a} are each independently a thermolabile substitution group.

Preferred embodiments of substituted NAD+ and derivatives thereof have the structure of Formulas IIA-IIE as follows:

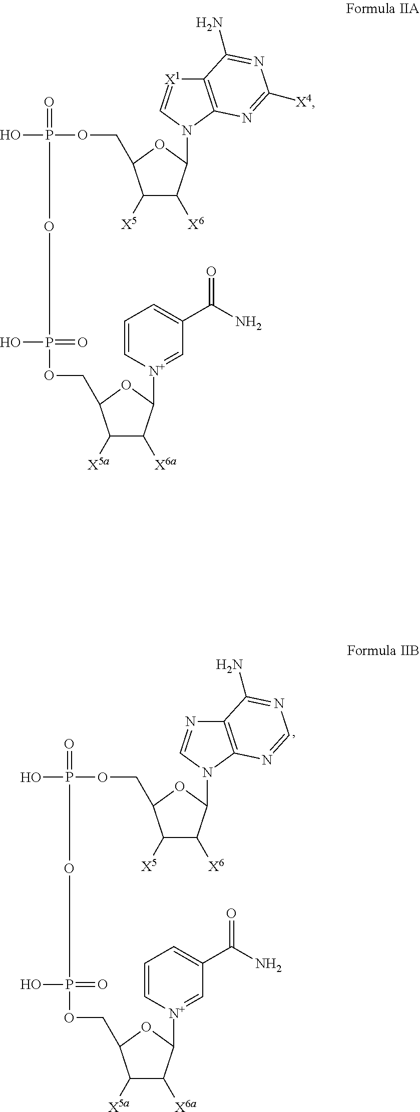

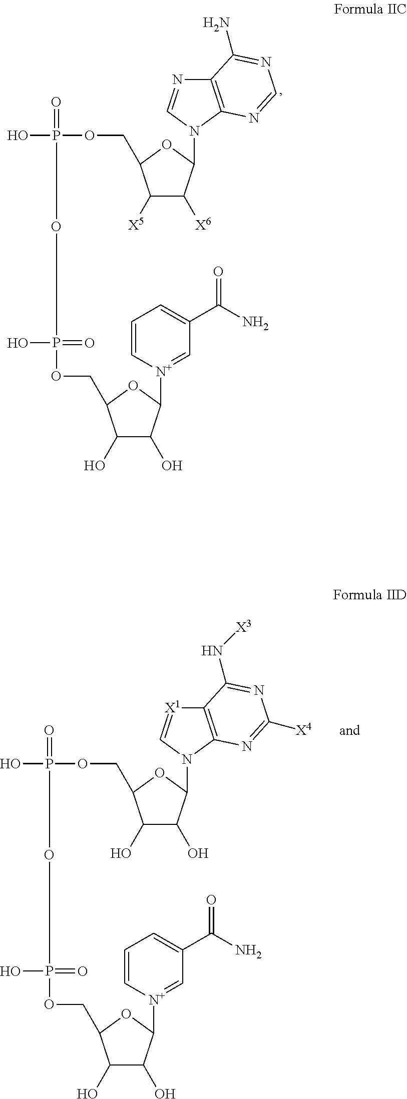

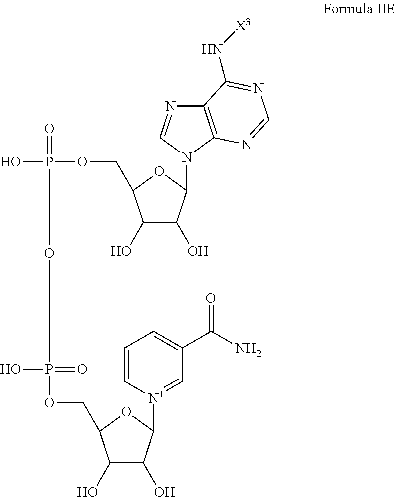

wherein:
X¹ and X⁴ are as defined in Formula II; and
at least one of X³, X⁵, X⁶, X^{5a} and X^{6a} is a thermolabile substitution group as defined in Formula II.
   In certain embodiments, both X⁵ and X⁶ are each a thermolabile substitution group as defined in Formula II.

Preferred embodiments of substituted NAD+ and derivatives thereof have the structures of the following Formulas:

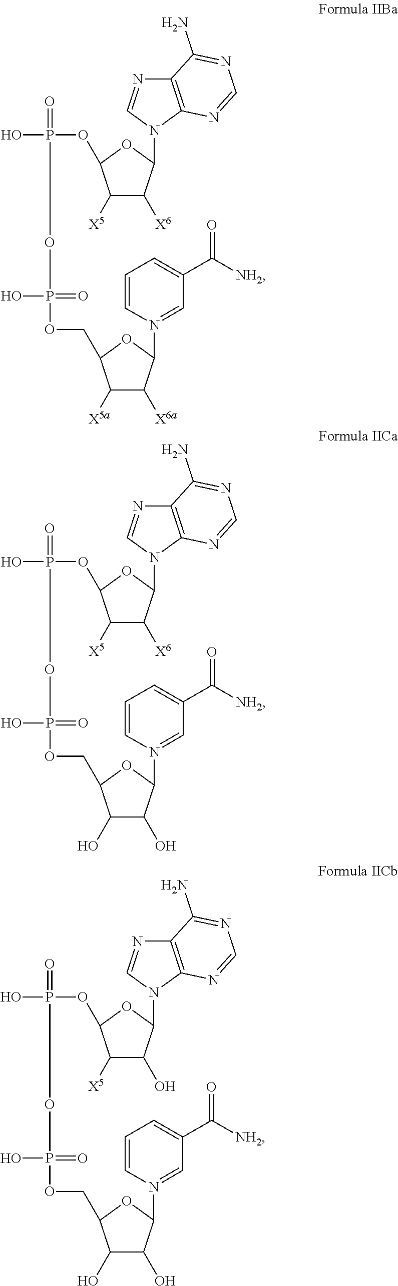

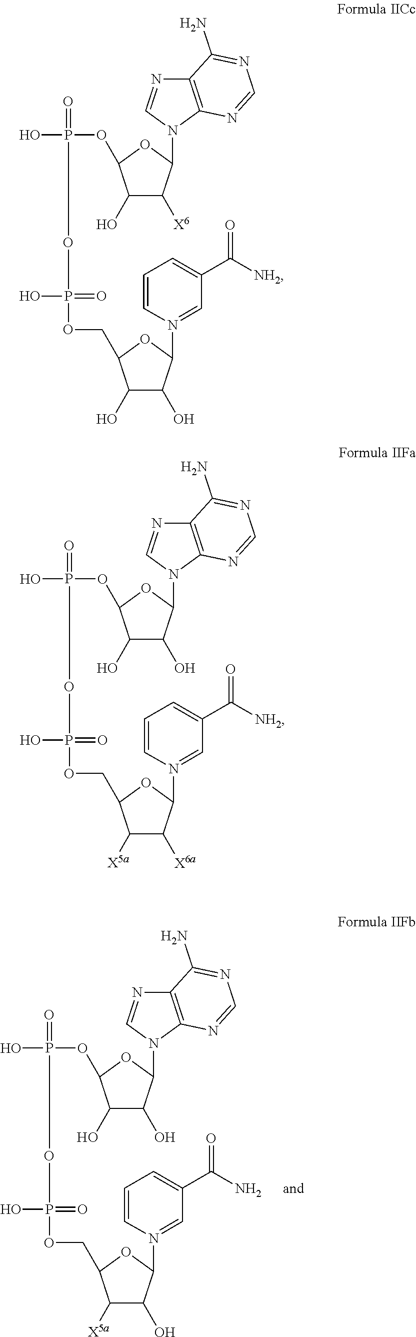

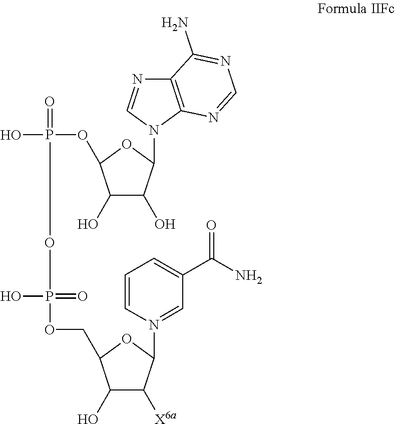

wherein:
at least one of X⁵, X⁶, X^{5a} and X^{6a} is a thermolabile substitution group independently selected from the group consisting of O-[4-methoxy]tetrahydropyranyl; O-tetrahydropyranyl; O-tetrahydrofuranyl; O-phenoxyacetyl; O-methoxyacetyl; O-(p-toluene)sulfonate; O-[4-methoxy]-tetrahydrothiopyranyl; O-tetrahydrothiopyranyl; O-[5-methyl]-tetrahydrofuranyl; O-[2-methyl, 4-methoxy]-tetrahydropyranyl; O-[5-methyl]-tetrahydropyranyl, O-tetrahydrothiofuranyl, O-2-[tert-butoxy]ethyl, O-2-[cyclohexoxy]ethyl, O-2-[isopropoxy]ethyl, O-2-[isobutoxy]ethyl, O-2-[ethoxy]ethyl, O-2-[propoxy]ethyl, O-2-[2-ethylhexoxy]ethyl, O-2-[butoxy]ethyl, O-2-[dodecoxy]ethyl, O-2-methyl-2-[ethoxy]ethyl, and O-2-[tert-pentoxy]ethyl.

Preferred embodiments of substituted NAD+ and derivatives thereof have the structure of Formula IIEa:

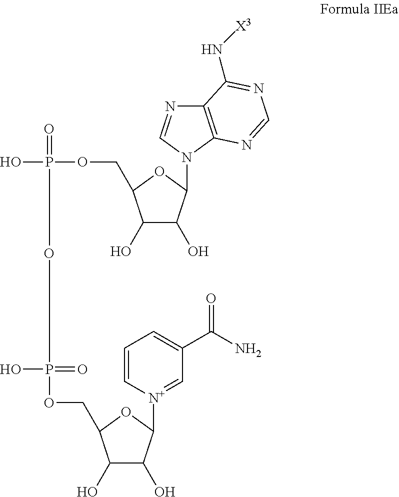

wherein:
X³ is a thermolabile substitution group selected from the group consisting of methoxycarbonyl, ethoxycarbonyl, 9-fluorenylmethoxycarbonyl, 2,2,2-trichloroethoxycarbonyl, 2-trimethylsylylethoxycarbonyl, tert-butoxycarbonyl, allyloxycarbonyl, benzyloxycarbonyl, phenyloxycarbonyl, p-nitrophenyloxycarbonyl, cyclohexyloxycarbonyl, phenoxyacetyl, methoxyacetyl, benzoyl, acetyl, dimethoxytrityl, monomethoxytrityl, trityl, N,N-dimethylaminomethylidene, N,N-diphenylaminomethylidene, and NN-dibenzylaminomethylidene.

### SAPs, SDPs and SADIs Carrying Thermally Labile Group on Internucleotide Linkages

In conjunction with any of the aspects, embodiments, compositions and methods disclosed herein, provided are substituted acceptor and donor oligonucleotide probes (SOPs), and SADI which provide utility in ligase mediated nucleic acid replication and/or ligation. The methods and compositions disclosed herein for ligase mediated nucleic acid replication are useful in applications that employ SOPs and/or SADIs and nucleic acid ligase. In some embodiments, SOPs and SADIs include a substitution group at an internucleotide linkage, for example, an internucleotide phosphotriester (PTE) group. SOPs and SADIs described herein can be used for ligase mediated nucleic acid replication applications, in particular for HS LCR.

In conjunction with any of the aspects, embodiments, compositions and methods disclosed herein, provided are SOPs and/or SADI that include one or more thermolabile substitution groups. The substitution group cleaves or dissociates during and after the initial heat denaturation step of the ligase reaction. In some embodiments, the substitution group includes one or more of the following chemical groups of Formula III, Formula IV and Formula V, further described herein.

In some embodiments, the thermolabile substitution group is attached to a SOP or a SADI creating, for example, a bulky PTE internucleotide linkage near the 3′ end of an acceptor probe or the 5′ end of the donor probe. The bulky PTE group impedes a ligase catalyzed phosphodiester bond formation between adjacent 3′-hydroxyl group of the acceptor probe and 5′-phosphoryl termini of the donor probe or adenylated donor intermediate on a nucleic acid template (e.g., RNA or DNA) prior to the initial heat denaturation step, Hot Start. The SOPs and SADIs disclosed herein can have a single substitution site or multiple substitution sites.

SOPs and SADIs as disclosed herein, can have two states. In the first state, the SOP or SADI is inactive due to the presence of a substitution group which impedes formation of ligation product prior the initial activation temperature is reached, often 95° C. (FIG. 2). Upon reaching the initial activation temperature, the SOP or SADI releases the substitution group by a thermally induced intra- or intermolecular fragmentation reaction and transforms to a second state. The second state of the SOP or SADI is equal to a corresponding unsubstituted oligonucleotide probe or adenylate-donor intermediate which has an unsubstituted internucleotide linkage and is usable by ligase. Partial or complete dissociation or cleavage of the substitution group preferably occurs after incubation of the SOP or SADI at approximately 95° C. for approximately 0.1-120 minutes. In certain embodiments, dissociation of the substitution group from the SOP or SADI occurs with respect to temperature and does not require other enzymes, chemicals, or specific ligation reaction conditions. Thermolabile substituted internucleotide linkages are described in Beaucage et. al., U.S. Pat. No. 6,762,298; Zon et al., US Patent application 20070281308; Lebedev, Current Protocols in Nucleic Acid Chemistry 2009, unit 4.35.; Ashrafi et al., Current Protocols in Molecular Biology 2009, unit 15.9. Hidalgo-Ashrafi, et al., BioTechniques 2009, 47(3): 789-90; Lebedev, et. al., Nucleic Acids Research 2008, 36(20): 131; Shum et al., Analytical Biochemistry 2009, 388: 266-272; and Hidalgo-Ashrafi et. al. BSC Molecular Biology 2009, 10: 113.

The SOPs and SADIs disclosed herein have significant advantages. The use of SOPs and/or SADIs can reduce the appearance of a “false positive” signal in ligase mediated nucleic acid detection applications and improves an overall performance of the applications. The end user can use the same protocols and methods already in use with unsubstituted oligonucleotide probes and/or adenylate-donor intermediates. SOPs and SADIs disclosed herein are compatible with existing systems and reagents, no additional enzymes or reagents are needed, but can be used.

Ligase mediated nucleic acid replication applications which employ oligonucleotide probes requiring high specificity and/or fidelity can be used with SOPs and SADIs of the present invention. The applications that involve nucleic acid amplification include but are not limited LCR, Gap-LCR, GEXL PCR, Ligase mediated PCR, HS LCR, ligase mediated PCR, multiplex LCR, quantitative LCR, Real Time LCR, nucleic acid sequencing or other nucleic acid amplification methods based on nucleic acid ligation known in the art.

In certain aspects, there are ligase based applications that do not require nucleic acid amplification. These applications can be used with the SOPs and SADIs disclosed herein. The applications include but not limited to ligase mediated gene assembly, ligase detection reaction, oligonucleotide ligation assay, HS LR, proximity ligation and other nucleic acid replication methods based on nucleic acid ligation known in the art.

Existing oligonucleotide synthesis methods can be used to synthesize the SOPs and SADIs disclosed herein. Other embodiments of the invention include commercial products for this technology including modified phosphoramidites, modified solid support for oligonucleotide synthesis, SOP and/or SADI sets for common targets, and custom synthesized SOP and SADI sequences.

In yet another aspect, provided are methods of manufacturing of SOPs and SADIs including performing oligonucleotide synthesis with modified phosphoramidites where the modified phosphoramidites comprise one or more of the following substitution groups of Formula III, Formula IV and Formula V, further described herein.

SAPs with Thermolabile Groups

In conjunction with any of the aspects, embodiments, compositions and methods disclosed herein, SAPs include one or more thermolabile substitution groups. In some embodiments, SAPs suitable for use with the methods and compositions described herein include modified oligonucleotides as described in the art, for example, PNA-DNA chimeric probes in Egholm, M., et al., U.S. Pat. No. 6,297,016.

In conjunction with any of the aspects, embodiments, compositions and methods disclosed herein, SAPs and derivatives thereof include compounds of Formula III:

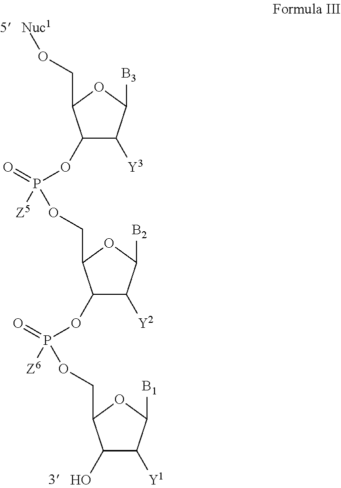

wherein:
B¹, B², and B³ are each independently selected from the group consisting of a substituted or non-substituted purine or pyrimidine, any aza or deaza derivative thereof, and any “universal base” or “degenerate base” of any nucleoside analog, which is preferably recognizable by a nucleic acid ligase and/or polymerase;
Nuc¹ is an oligonucleotide;
Y¹, Y², and Y³ are each independently selected from the group consisting of H, F, OH and OCH₃;
at least one of Z⁵ and Z⁶ is independently a thermolabile substitution group of structure U-Φ U is selected from group consisting of O, S, Se, NR¹¹, and CR¹¹R¹²;
R¹¹ and R¹² are each independently hydrogen or optionally substituted straight or branched hydrocarbyl having from 1-20 carbon atoms, wherein each may independently include at least one substituent selected from halo, oxo, hydroxyl, alkoxy, aryloxy, amino, amido or a detectable label; and
Φ is one or more groups selected from the group consisting of:

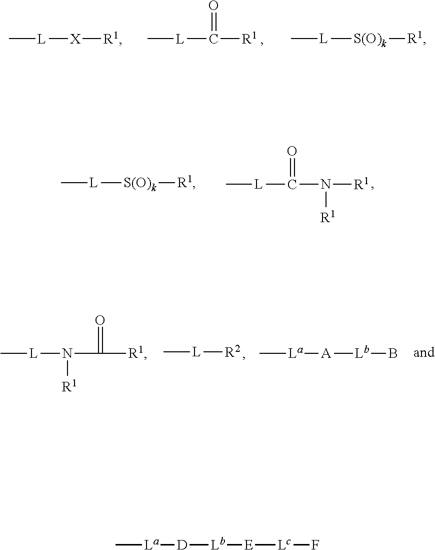

wherein:
L is a straight or branched hydrocarbylene group having between 1-10 carbon atoms;
X is O, S, S(O), S(O)₂, C(O), C(S) or C(O)NH; and
R¹ is hydrogen or a straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl;
k is an integer from 0-2;
R² is an optionally substituted carbocycle, heterocycle, aryl or heteroaryl having between 5-10 atoms;
L^{a}, L^{b} and L^{c} are each independently selected from a bond or a straight or branched hydrocarbylene group having between 1-8 carbon atoms;
A is O, S, S(O), S(O)₂, Se, CR³R⁴, NR³, C(O), C(S) or CNR³;
B is C(O)R³, C(S)R³, C(O)NR³R⁴, OR³ or SR³; and
R³ and R⁴ are each independently hydrogen or straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl; and
D is O, S, S(O), S(O)₂, CR⁵R⁶ or NR^{S};
E is O, S, S(O), S(O)₂, CR⁵R⁶ or NR⁶;
F is hydrogen, C(O)R⁷, C(S)R⁷, C(O)NR⁷R⁸, OR⁷ or SR⁷;
R⁵ and R⁶ can each independently be hydrogen, aryl, alkyl, halo, oxo, hydroxyl, alkoxy, aryloxy or amino, or R⁵ and R⁶ can cooperate to form a mono or bicyclic ring consisting 5-10 atoms and including D, R⁵, R⁶, E and L^{b}, provided that when R⁵ and R⁶ cooperate to form a ring; and
R⁷ and R⁸ are each independently selected from the group consisting of aryl, alkyl, halo, oxo, hydroxyl, alkoxy, aryloxy, amino, amido, optionally substituted cycloalkyl, optionally substituted heterocycloalkyl, optionally substituted aryl, optionally substituted aryloxy, and optionally substituted heteroaryl.

Preferred embodiments of SAPs and derivatives thereof have the structure of Formula IIIA:

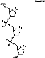

wherein:
Nuc¹ is a deoxyribooligonucleotide;
B₁, B₂ and B₃ are each nucleoside bases independently selected from the group consisting of adenine, guanine, thymine, cytosine, their derivatives and analogs; and
Z⁶ is a thermolabile substitution group of the structure O-Φ,
where Φ is one or more groups selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

In some embodiments, Φ, comprises one or more chemical formulas selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

SDPs with Thermolabile Groups

In conjunction with any of the aspects, embodiments, compositions and methods disclosed herein, SDPs include one or more thermolabile substitution groups. In some embodiments, SDPs suitable for use with the methods and compositions described herein may be used in combination with substitutions described in the art, for example, use of 5′-thiophosphates instead of 5′-phosphate in the donor strand (Bandaru, R., et al. U.S. Pat. Nos. 6,811,986 and 6,635,425).

In conjunction with any of the aspects, embodiments, compositions and methods disclosed herein, SDPs and derivatives thereof include compounds of Formula IV:

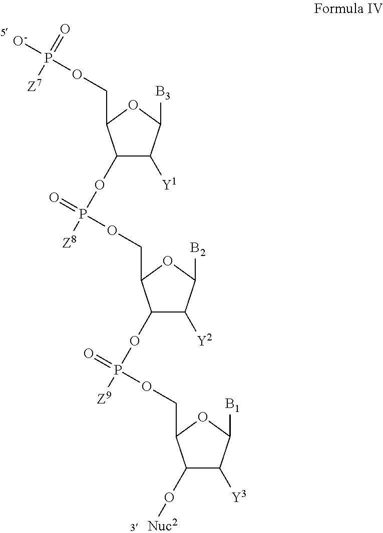

wherein:
B¹, B², and B³ are each independently selected from the group consisting of a substituted or non-substituted purine or pyrimidine, any aza or deaza derivative thereof, and any “universal base” or “degenerate base” of any nucleoside analog, which is preferably recognizable by a nucleic acid ligase and/or polymerase;
Nuc² is an oligonucleotide;
Y¹, Y², and Y³ are each independently selected from the group consisting of H, F, OH and OCH₃;
Z⁷, Z⁸, and Z⁹ are each independently selected from group consisting of OH and SH
   wherein Z⁸ and Z⁹ are each optionally a thermolabile substitution group;
      at least one of Z⁸ and Z⁹ is independently a thermolabile substitution group of structure U-Φ U is selected from group consisting of O, S, Se, NR¹¹, and CR¹¹R¹²;
R¹¹ and R¹² are each independently hydrogen or optionally substituted straight or branched hydrocarbyl having from 1-20 carbon atoms, wherein each may independently include at least one substituent selected from halo, oxo, hydroxyl, alkoxy, aryloxy, amino, amido or a detectable label; and
Φ is one or more groups selected from the group consisting of:

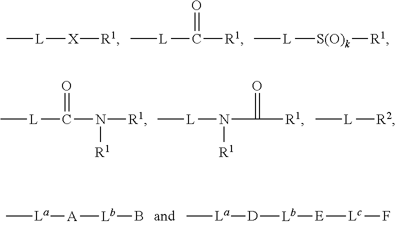

wherein:
L is a straight or branched hydrocarbylene group having between 1-10 carbon atoms;
X is O, S, S(O), S(O)₂, C(O), C(S) or C(O)NH; and
R¹ is hydrogen or a straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl;
k is an integer from 0-2;
R² is an optionally substituted carbocycle, heterocycle, aryl or heteroaryl having between 5-10 atoms;
L^{a}, L^{b} and L^{c} are each independently selected from a bond or a straight or branched hydrocarbylene group having between 1-8 carbon atoms;
A is O, S, S(O), S(O)₂, Se, CR³R⁴, NR³, C(O), C(S) or CNR³;
B is C(O)R³, C(S)R³, C(O)NR³R⁴, OR³ or SR³; and
R³ and R⁴ are each independently hydrogen or straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl; and
D is O, S, S(O), S(O)₂, CR⁵R⁶, or NR⁵;
E is O, S, S(O), S(O)₂, CR⁵R⁶, or NR⁶;
F is hydrogen, C(O)R⁷, C(S)R⁷, C(O)NR⁷R⁸, OR⁷ or SR⁷;
R⁵ and R⁶ can each independently be hydrogen, aryl, alkyl, halo, oxo, hydroxyl, alkoxy, aryloxy or amino, or R⁵ and R⁶ can cooperate to form a mono or bicyclic ring consisting 5-10 atoms and including D, R⁵, R⁶, E and L^{b}, provided that when R⁵ and R⁶ cooperate to form a ring; and
R⁷ and R⁸ are each independently selected from the group consisting of aryl, alkyl, halo, oxo, hydroxyl, alkoxy, aryloxy, amino, amido, optionally substituted cycloalkyl, optionally substituted heterocycloalkyl, optionally substituted aryl, optionally substituted aryloxy, and optionally substituted heteroaryl.

Preferred embodiments of SDPs and derivatives thereof have the structure of Formula IVA:

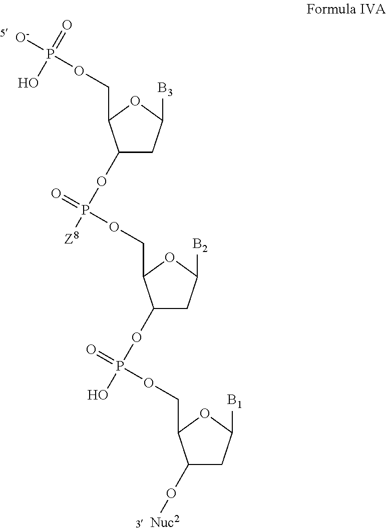

wherein:
Nuc² is a deoxyribooligonucleotide;
B₁, B₂ and B₃ are each nucleoside bases independently selected from the group consisting of adenine, guanine, thymine, cytosine, their derivatives and analogs; and
Z⁸ is a thermolabile substitution group of the structure O-Φ,
wherein Φ is one or more groups selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

In preferred embodiments, thermolabile substitution group, Φ, comprises one or more chemical formulas selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

SADIs with Thermolabile Groups

In conjunction with any of the aspects, embodiments, compositions and methods disclosed herein, SADIs include one or more thermolabile substitution groups attached to adenylate residue and/or oligonucleotide moiety. In some embodiments, SADIs suitable for use with the methods and compositions described herein may be used in combination with chemical substitutions described in the art, for example, use of 5′-thiophosphates instead of 5′-phosphate in the donor strand (Bandaru, R., et al. U.S. Pat. Nos. 6,811,986 and 6,635,425).

In conjunction with any of the aspects, embodiments, compositions and methods disclosed herein, SADIs and derivatives thereof include compounds of Formula V:

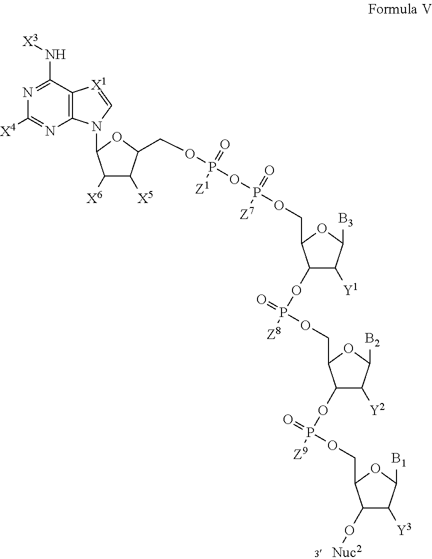

wherein:
B¹, B², and B³ are each independently selected from the group consisting of a substituted or non-substituted purine or pyrimidine, any aza or deaza derivative thereof, and any “universal base” or “degenerate base” of any nucleoside analog, which is preferably recognizable by a nucleic acid ligase and/or polymerase;
Nuc² is an oligonucleotide;
Y¹, Y², and Y³ are each independently selected from the group consisting of H, OH, F and OCH₃;
X¹ is selected from the group consisting of C—X² and N;
X² is selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
Z¹ and Z⁷ are each independently selected from the group consisting of OH, OR¹, SH, SR¹, CH₃, BH₃⁻, NH₂, NHR¹, NR¹R², F, phosphate, substituted phosphate, substituted polyphosphate, substituted phosphonate, sulfate, sulphonate, O-acyl, S-acyl, NH-acyl, and NR¹-acyl;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl,
   wherein X³ is optionally a thermolabile substitution group;
X⁴ is selected from the group consisting of hydrogen, NH₂, NHR¹, OH, OR¹, SH, SR¹ and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
      each X⁵ and X⁶ is independently selected from the group consisting of OH,

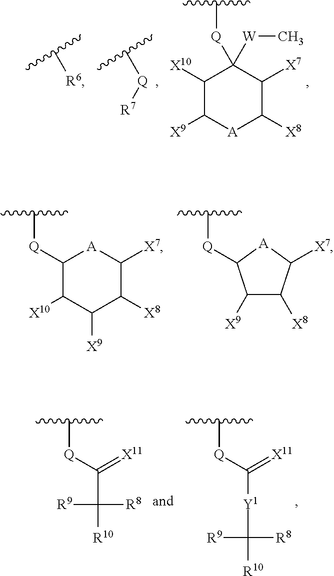

wherein X⁵ and X⁶ are each optionally a thermolabile substitution group;
Q is selected from group consisting O, S, NH, NR¹, NOR¹, CHR¹, and CR¹R²;
R⁶ is selected from the group consisting of inorganic acid residue, or derivative thereof, with the exception of carbonic acid, where the derivatives may include but are not limited to halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R⁷, R⁸, R⁹ and R¹⁰ is independently selected from the group consisting of hydrogen, and a straight or branched optionally substituted hydrocarbyl group having from 1-20 carbon atoms, preferably 1-10 carbon atoms, preferably 1-6 carbon atoms,
   wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting of O, S, NH, NR¹, NOR¹, CHR¹, and CR¹R²;
each A, Y¹ and W is independently selected from the group consisting of O, S, NH, NR¹, NOR¹, CHR¹, and CR¹R²;
R¹¹ and R¹² are each independently hydrogen or optionally substituted straight or branched hydrocarbyl having from 1-20 carbon atoms,
   wherein each may independently include at least one substituent selected from halo, oxo, hydroxyl, alkoxy, aryloxy, amino, amido or a detectable label; and
Z⁸ and Z⁹ are each independently OH, SH or a thermolabile substitution group having the structure U-Φ;
   wherein U is selected from group consisting of O, S, Se, NR¹¹, and CR¹¹R¹²; and
   Φ is one or more substitution groups selected from the group consisting of:

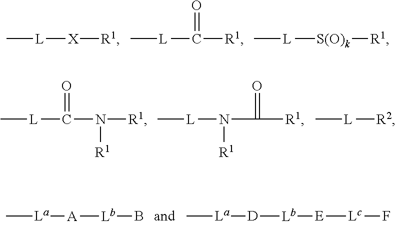

wherein:
L is a straight or branched hydrocarbylene group having between 1-10 carbon atoms;
X is O, S, S(O), S(O)₂, C(O), C(S) or C(O)NH; and
R¹ is hydrogen or a straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl;
k is an integer from 0-2;
R² is an optionally substituted carbocycle, heterocycle, aryl or heteroaryl having between 5-10 atoms;
L^{a}, L^{b} and L^{c} are each independently selected from a bond or a straight or branched hydrocarbylene group having between 1-8 carbon atoms;
A is O, S, S(O), S(O)₂, Se, CR³R⁴, NR³, C(O), C(S) or CNR³;
B is C(O)R³, C(S)R³, C(O)NR³R⁴, OR³ or SR³; and
R³ and R⁴ are each independently hydrogen or straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl; and
D is O, S, S(O), S(O)₂, CR⁵R⁶, and NR^{S};
E is O, S, S(O), S(O)₂, CR⁵R⁶, and NR⁶;
F is hydrogen, C(O)R⁷, C(S)R⁷, C(O)NR⁷R⁸, OR⁷ and SR⁷;
R⁵ and R⁶ can each independently be hydrogen, aryl, alkyl, halo, oxo, hydroxyl, alkoxy, aryloxy or amino, or R⁵ and R⁶ can cooperate to form a mono or bicyclic ring consisting 5-10 atoms and including D, R⁵, R⁶, E and L^{b}, provided that when R⁵ and R⁶ cooperate to form a ring; and
R⁷ and R⁸ are each independently selected from the group consisting of aryl, alkyl, halo, oxo, hydroxyl, alkoxy, aryloxy, amino, amido, optionally substituted cycloalkyl, optionally substituted heterocycloalkyl, optionally substituted aryl, optionally substituted aryloxy, and optionally substituted heteroaryl;
wherein at least one of Z⁸, Z⁹, X³, X⁵ and X⁶ are each independently a thermolabile substitution group.

Preferred embodiments of SADIs and derivatives thereof have the structure of Formula VA:

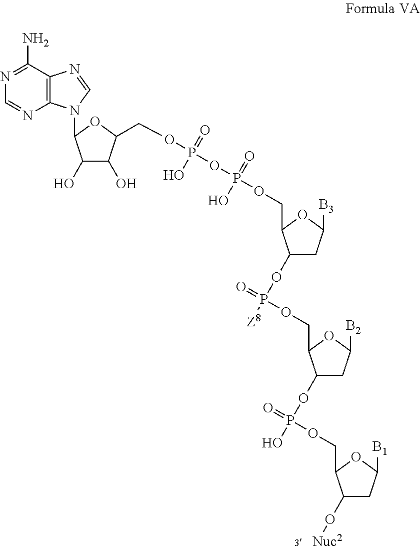

wherein:
Nuc² is a deoxyribooligonucleotide;
B₁, B₂ and B₃ are each nucleoside bases independently selected from the group consisting of adenine, guanine, thymine, cytosine, their derivatives and analogs; and
Z⁸ is a thermolabile substitution group of the structure O-Φ,
   where Φ is one or more groups selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

Preferred embodiments of SADIs and derivatives thereof have the structures of the following Formulas:

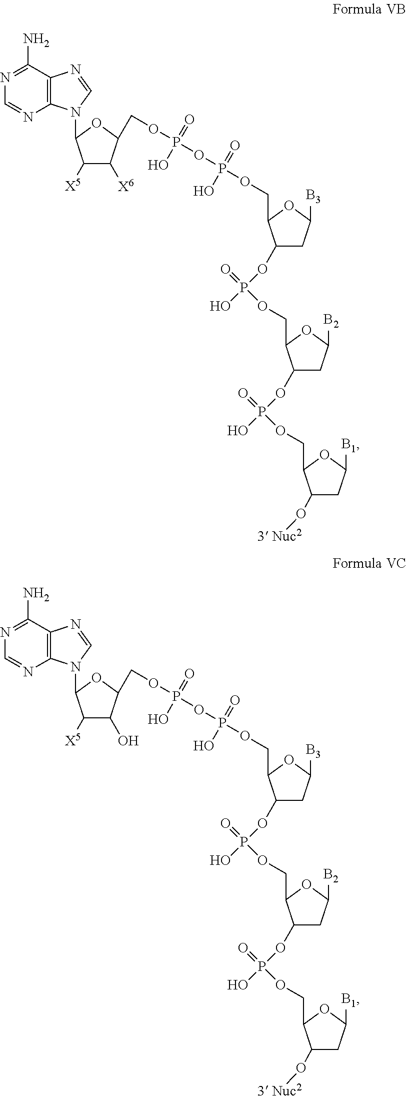

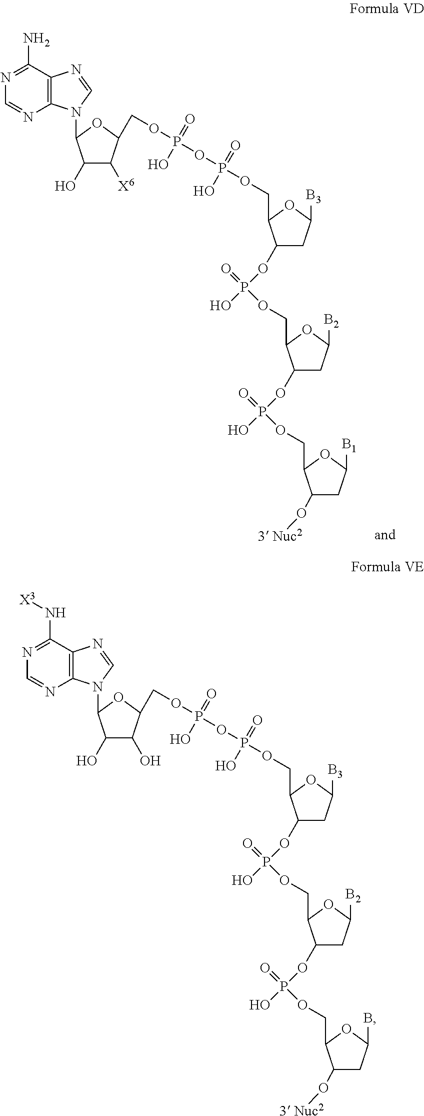

wherein:
Nuc² is a deoxyribooligonucleotide;
B₁, B₂ and B₃ are each nucleoside bases independently selected from the group consisting of adenine, guanine, thymine, cytosine, their derivatives and analogs; and
X³ is a thermolabile substitution group selected from the group consisting of methoxycarbonyl, ethoxycarbonyl, 9-fluorenylmethoxycarbonyl, 2,2,2-trichloroethoxycarbonyl, 2-trimethylsylylethoxycarbonyl, tert-butoxycarbonyl, allyloxycarbonyl, benzyloxycarbonyl, phenyloxycarbonyl, p-nitrophenyloxycarbonyl, cyclohexyloxycarbonyl, phenoxyacetyl, methoxyacetyl, benzoyl, acetyl, dimethoxytrityl, monomethoxytrityl, trityl, N,N-dimethylaminomethylidene, N,N-diphenylaminomethylidene, and NN-dibenzylaminomethylidene.
X⁵ and X⁶ are each thermolabile substitution groups independently selected from the group consisting of O-[4-methoxy]tetrahydropyranyl; O-tetrahydropyranyl; O-tetrahydrofuranyl; O-phenoxyacetyl; O-methoxyacetyl; O-(p-toluene)sulfonate; O-[4-methoxy]-tetrahydrothiopyranyl; O-tetrahydrothiopyranyl; O-[5-methyl]-tetrahydrofuranyl; O-[2-methyl, 4-methoxy]-tetrahydropyranyl; O-[5-methyl]-tetrahydropyranyl, O-tetrahydrothiofuranyl, O-2-[tert-butoxy]ethyl, O-2-[cyclohexoxy]ethyl, O-2-[isopropoxy]ethyl, O-2-[isobutoxy]ethyl, O-2-[ethoxy]ethyl, O-2-[propoxy]ethyl, O-2-[2-ethylhexoxy]ethyl, O-2-[butoxy]ethyl, O-2-[dodecoxy]ethyl, O-2-methyl-2-[ethoxy]ethyl, and O-2-[tert-pentoxy]ethyl.

In certain embodiments, SAP, SDP and/or SADI include a thermolabile phosphotriester internucleotide linkage. In certain embodiments, SAP includes two or more thermolabile substitution groups at the n−1, n−2, n−3, n−4, n−5 or n−6 position; wherein “n−1” position is the 3′ terminal internucleotide linkage or linkages. In certain embodiments, SDP or SADI include two or more thermolabile substitution groups at the n+1, n+2, n+3, n+4, n+5 or n+6 position; wherein “n+1” is the 5′ terminal internucleotide linkage. The ligation junction is defined as position “n”.

In certain embodiments, a thremolabile substitution group impairs hybridization of the SAP, SDP or SADI to a nucleic acid sequence prior to Hot Start activation step. In certain embodiments the presence of a thermolabile substitution group inhibits or impedes ligase reaction of the SAP, SDP or SADI.

Adenylate-Donor Intermediate with Thermally Labile Group on an AMP Fragment

In conjunction with any of the aspects, embodiments, compositions and methods disclosed herein, a substituted adenylate moiety of a SC is transferred by ligase onto the 5′-phosphate end of a substituted or unsubstituted donor probe forming yet another type of SADI. In certain embodiments, SADI is inactive and cannot be used by ligase due to the presence of a substitution group on the adenylate moiety. Upon reaching an initial denaturation temperature, often 95° C., inactive SADI can be converted to an active state by thermally induced intra- and/or intermolecular cleavage of any or all thermolabile substitutions at adenylate residue. This active state of the SADI corresponds to an unsubstituted adenylate-donor intermediate which possesses substrate properties for nucleic acid ligase and supports ligase mediated nucleic acid replication (FIG. 2).

In certain embodiments a pre-synthesized substituted adenylate residue can be chemically attached to the 5′-phosphate end of a substituted or unsubstituted donor probe resulting in yet another type of SADI. In certain embodiments, SADI is inactive and cannot be used by ligase due to the presence of substitution group on adenylate moiety. Upon reaching an initial denaturation temperature, often 95° C., inactive SADI can be converted to an active state by thermally induced intra- and/or intermolecular cleavage of any or all thermolabile substitutions at adenylate residue. This active state of the SADI corresponds to an unsubstituted adenylate-donor intermediate which possesses substrate properties for nucleic acid ligase and supports ligase mediated nucleic acid replication (FIG. 2).

In certain embodiments, a SADI including a thermally labile substitution group at an adenylate residue, may carry an additional thermolabile substitution group at a donor oligonucleotide moiety.

In certain embodiments, a SADI may include additional thermostable substitutions or modifications in the adenylate or oligonucleotide portions of the adenylate-donor intermediate, for example, nucleoside residues with modified sugar, base, (5′-3′)-internucleotide linkages, or any combination thereof in addition to containing an 5′-adenylate with thermolabile substitution group or groups. In certain embodiments, the SADI contains a thermolabile substitution group at 5′-adenylate residue depicted in Formula V further described herein.

In certain embodiments partial or complete conversion of SADI from inactive state to active state occurs after incubation at approximately 95° C. for approximately 0.1-120 minutes. In preferred embodiments, conversion of SADI from inactive state to active state occurs with respect to temperature and does not require enzymes, additional chemicals, or modified reaction conditions other than those normally used in ligase based replication reactions.

In certain embodiments, methods of synthesis of SADI derivatives are disclosed herein using chemical and/or enzymatic procedures known in art.

Common Properties of SLCs with Thermolabile Groups

In conjunction with any of the aspects, embodiments, compositions and methods disclosed herein, ligase mediated nucleic acid replication are useful in applications that employ synthetic and/or natural SC, unsubstituted cofactors, SOPs, unsubstituted oligonucleotide probes, SADI, unsubstituted adenylated donor intermediates and ligase for ligation of nucleic acid. The SLCs may optionally have additional sites at sugar, nucleoside base or phosphate moiety that are modified with thermostable groups. Standard chemical and enzymatic synthesis methods can be used to synthesize the SLCs of the methods and compositions provided herein.

In certain embodiments, the SLC may have one or more detectable labels. Thus, following ligation, a labeled nucleic acid or any ligase intermediate may be identified by size, mass, affinity capture and/or color. Detectable labels include, but are not limited to, chromophores, fluorescent dyes, enzymes, antigens, heavy metals, magnetic probes, phosphorescent groups, radioactive materials, chemiluminescent moieties and electrochemical detecting moieties. The detectable label is preferably a fluorescent dye; a preferable affinity capture label is biotin.

In certain embodiments, at least one SOP or SADI in a replication or amplification reaction is labeled with a detectable label. Thus, following ligation, the target segment can be identified by size, affinity capture or color. The detectable label is preferably a fluorescent dye. In some embodiments, different pairs of probes or cofactors in a multiplex LCR may be labeled with different distinguishable detectable labels. In other embodiments, the acceptor probe is labeled with one detectable label, while the donor probe or SADI is labeled with a different detectable label. Use of different detectable labels is useful in multiplex assays for discriminating between ligated products which are of the same length or are very similar in length. Thus, in certain embodiments, at least two different fluorescent dyes are used to label different probes used in a single ligase mediated replication reaction.

Other therolabile protecting groups suitable for substitution groups of the SLC compositions and methods described herein (e.g., 2′,3′ substitutions for ATP; internucleotide linkages substitutions for oligonucleotides) have been described in literature for use in the nucleotide and oligonucleotide synthesis processes. See Koukhareva et al., Analytical Chemistry 81, 4955-4962 (2009); Lebedev et al., US Patent Application 20100003724; Le, et al., BioTechniques 2009, 47(5): 972-3; Le et al., BioTechniques 2009, 47(3): 789-90; Koukhareva et al., Nucleic Acids Symposium Series 2008, No. 52: 259-260; Koukhareva et al., Collection Symposium Series 2008, Academy of Sciences of the Czech Republic, 10: 259-263; Grajkowski, et al., Org. Lett., 1287-1290 (2001); Wilk, A., et al., Tetrahedron Lett., 5635-5439 (2001); Wilk, A., et al., J. Org. Chem., 6430-6438 (2002); Cieslak, J., et al., J. Org. Chem., 10123-10129 (2003); Cieslak, J., et al., J. Org. Chem., 2509-2515 (2004); Beaucage, et al., U.S. Patent Appl. No. 20050020827; Beaucage, et al., U.S. Pat. No. 6,762,298; Beaucage et al., U.S. Pat. No. 6,762,298; Zon et al., US Patent application 20070281308; Lebedev, Current Protocols in Nucleic Acid Chemistry 2009, 4.35.; Ashrafi et al., Current Protocols in Molecular Biology 2009, 15.9; Hidalgo Ashrafi et al., BioTechniques 47, 789-90 (2009); Lebedev, et al., Nucleic Acids Research 36, e131 (2008); Shum et al., Analytical Biochemistry 388, 266-272 (2009); Hidalgo Ashrafi et. al. BMC Molecular Biology 10, 113 (2009). Non thermolabile substitution groups for sugar, base and phosphates of nucleosides, nucleotides and oligonucleotides of the compositions and methods provided herein are described, for example, in Greene, T. W. et al., P.G.M., Protective groups in organic synthesis, John Wiley & Sons, Inc. (1999).

The methods and compositions herein are directed to the use of heat activatable SLCs containing thermolabile substitution groups in temperature dependent ligase mediated nucleic acid replication reaction. Any thermolabile substitution group of SLC that accomplishes the purposes of the methods and compositions provided herein may be utilized. The substitution group should be one that leads to reduction or elimination of undesired formation of ligation product at low stringency conditions of ligation reaction in which the SLC is to be employed.

In another aspect, provided herein are methods of synthesis of SLCs having a chemical structure as depicted in Formulas I-V further described herein. The thermolabile substitution group can be integrated into a ligase cofactor, acceptor, donor or adenylate-donor intermediate by using existing synthetic or enzymatic methods. The SLCs of the methods and compositions provided herein may be synthesized by any methods well-known in the art. Following synthesis and purification of a SLC, several different procedures may be utilized to determine the acceptability of the SAP in terms of structure and purity. Examples of such procedures are Nuclear Magnetic Resonance Spectroscopy, Mass Spectrometry, Fluorescent Spectroscopy, Ultra Violet Spectroscopy, High Performance Liquid Chromatography. These procedures are well known to those skilled in the art. Current methods employed for separation, purification and analysis in the art are applicable to the SLCs of the methods and compositions provided herein as well.

The SLCs of the methods and compositions provided herein preferably have no efficacy or reduced efficacy in ligase mediated replication of nucleic acid at ambient conditions prior to Hot Start activation step, as compared to the unsubstituted ligase components. The replication reaction with SLC is considered impeded when, it is at least 50% less efficacious as replication reaction as compared to its corresponding unsubstituted ligase component, preferably at least 60% less efficacious, preferably at least 70% less efficacious, more preferably at least 80% less efficacious, more preferably at least 90% less efficacious, more preferably at least 95% less efficacious, more preferably at least 99% less efficacious and most preferably 100% less efficacious at ligase mediated replication reaction than its corresponding unsubstituted ligase component. One of ordinary skill in the art is able to readily determine the level of ligation activity and efficacy for SLC before Hot Star activation. After Hot Start activation, the use SLCs of the methods and compositions provided herein preferably results, in at least about the same efficacy for ligase mediated replication of nucleic acid as compared to the unsubstituted or natural ligase component.

In some embodiments, the use of heat activatable SLCs improves specificity of ligase reaction compared with the corresponding unsubstituted or natural ligase components. Improving ligation refers to the ability of the ligase reaction in conjunction with Hot Start activation to discriminate between matched and mismatched nucleic acid and sequences reduce or eliminate template independent ligation, in particular blunt-ended ligation. Preferably, the use of the SLCs without heat activation step prevents or impedes any non-templated ligation when nucleic acid target is either present or absent in reaction mixture. Most preferably, the use of the SLC without heat activation step prevents or impedes ligation when there is a mismatch or mismatches in the donor and/or acceptor strands as compared to the target nucleic acid sequence. In other embodiments, the use of SLCs with heat activation step improves ligation specificity in favor of perfectly matched complexes when mismatched (or non-complementary) nucleic acid and matched (complementary) nucleic acid sequences are present simultaneously. In preferred embodiments, Hot Start ligation with a SLC improves ligation specificity by at least 1%, at least 2%, at least 5%, at least 10%, at least 15%, at least 20%, at least 30%, at least 50%, at least 70%, at least 90%, at least 100%, at least 200%, at least 500%, at least 1000%, at least 5000%, at least 10000%, at least 50000% or at least 100000% or more.

In some ligation reactions, not all ligase components in the ligase reaction mixture will contain a thermolabile substitution group. Preferably, even a mixture of both substituted and unsubstituted ligase components improves efficacy and specificity of Hot Start ligation in a mixed population, as compared to not using SLC at all. Preferably, prior to incubation at an initial denaturation temperature, or Hot Start, SLC molecules make up at least 1% of total ligase component molecules, preferably at least 5% of total ligase component molecules, preferably at least 10% of total ligase component molecules, preferably at least 25% of total ligase component molecules, preferably at least 50% of total ligase component molecules, preferably at least 75% of total ligase component molecules and preferably at least 90% of total ligase component molecules, preferably at least 95% of total ligase component molecules, preferably at least 98% of total ligase component molecules, more preferably at least 99% of total ligase component molecules, and most preferably 100% of total SLC molecules. In another embodiment, two, three, four or more types of SLC molecules may be employed in a ligation reaction.

In some embodiments, the SLCs may have only one thermolabile substitution group. In other embodiments, the SLCs may contain more than one thermolabile substitution group at the nucleoside base, at internucleotide phosphate, triphosphate or pyrophosphate moieties, at nucleoside sugar, or combinations of any two or more thereof. In other embodiments, the SLCs may contain more than one type of thermolabile substitution group. The SLCs may have the chemical structure of Formulas I-V described herein.

In some embodiments, the SLCs may have thermolabile substitution group and additional thermostable substitution group or modification. In some embodiments, the SLCs may contain more than one thermolabile substitution group. The SLCs may have the chemical structure of Formulas I-V described herein.

In some embodiments, only one type SLC is present in the ligation reaction. In other embodiments different types of SLC may be present in the same ligation reaction. In certain embodiments, two or more types of SLCs may be present in the same ligation reaction. In certain embodiments, three or more types of SLCs may be present in the same ligation reaction. In certain embodiment, four or more types of SLCs may be present in the same ligation reaction.

### Combinations of SCs, SAPs, SADs and SADIs Carrying Thermolabile Groups

Certain embodiments and embodiments of the compositions and methods provided herein include the use of combinations of SCs, SAPs, SDPs and SADIs. Any possible combination of two or more may be used. In some embodiments, more than one type of SC, SAP, SDP and SADI may be used.

Exemplary combinations include combinations of two or more of SCs, SAPs, SDPs and SADIs selected from the Formulas I, II, III, IV and V.

As used herein, the term “ligase cofactor” refers to chemical compound (e.g., ATP or NAD+) that reacts with the e-amino group of lysine of the nucleic acid ligase forming an activated adenylate-ligase with covalent phosphoramidate linkage (e.g., as shown in FIG. 4). In preferred embodiments, the ligase cofactor is ATP, NAD+ or GTP. Generally ligases are ATP-dependent or NAD+-dependent.

As used herein, the term “activatable cofactor” refers to a SC that has one or more thermolabile substitution groups at a sugar and/or base and/or phosphate group. In some embodiments, the activatable cofactor does not support two-step ligase-assisted transfer of its adenylyl moiety onto the 5′-phosphate end of the donor probe prior to a heat activation step, e.g., Hot Start (FIG. 2). An activatable cofactor of the methods and compositions provided herein has two states. The activatable cofactor is in an inactive state due to the presence of one or more substitution group and it does not support ligase reaction. Upon reaching an initial denaturation temperature, e.g., 95° C., inactive activatable cofactor can be converted to an active state by thermally induced intra- and/or intermolecular cleavage of any or all thermolabile substitution groups. This active state of the activatable cofactor corresponds to a natural or unsubstituted cofactor, or a functional derivative thereof, which possesses cofactor properties for nucleic acid ligase and supports ligase mediated nucleic acid replication.

As used herein, the term “substituted cofactor”, “SC” or “substituted ligase cofactor” refers to a ligase cofactor with a thermolabile substitution group attached. Preferably, the substitution group impedes the ability of the cofactor to support ligase reaction between donor and acceptor probes. In preferred embodiments, the substituted cofactor is substituted ATP or substituted NAD+. In some embodiments, a substituted cofactor has more than one thermolabile substitution group. Substituted cofactors include those depicted herein, for example, Formulas I and II.

As used herein, the term “sugar substituted cofactor” refers to a ligase cofactor (e.g., ATP or NAD+) with a thermolabile substitution group attached to a sugar moiety as depicted herein, for example, Formulas I and II.

As used herein, the term “base substituted cofactor” refers to a ligase cofactor (e.g., ATP or NAD+) with thermolabile substitution group attached to an adenine base moiety as depicted herein, for example, Formulas I and II.

As used herein, the term “polyphosphate substituted ligase cofactor” refers to a ligase cofactor with a thermolabile substitution group attached to a 5′-triphosphate moiety of ATP or pyrophosphate moiety of NAD+ as depicted herein, for example, Formulas I and II.

As used herein, the term “unsubstituted ligase cofactor” or “natural ligase cofactor” in relation to a “substituted ligase cofactor” refers to the corresponding natural or ligase cofactor without the substitution group, or equivalent thereof. For example, the natural or unsubstituted ligase cofactor relative to substituted ATP is ATP.

As used herein, the term “acceptor,” “acceptor probe,” “acceptor polynucleotide probe” or “acceptor oligonucleotide probe” refers to an oligonucleotide or polynucleotide with a 3′ OH group capable of being ligated to a donor probe. An acceptor probe may be suitable for ligation when hybridized in close proximity to a donor probe or adenylate-donor intermediate on a complementary target nucleic acid in conditions suitable for nucleic acid ligation; preferably an acceptor probe hybridizes adjacent to donor probe or adenylate-donor intermediate on a complementary target nucleic acid. In some embodiments, an acceptor probe has at least one nucleic acid site that is not complementary (mismatch) to a target nucleic acid. In particular embodiments, the mismatch is at a nucleotide position of interest (e.g., SNP site). Additional alternative acceptor probes suitable for the methods and compositions provided herein include, but are not limited to, substituted, unsubstituted and modified nucleic acids, substituted, unsubstituted and modified ribonucleotides, substituted, unsubstituted and modified deoxyribonucleotides, substituted, unsubstituted and modified deoxyribooligonucleotides, substituted, unsubstituted and modified ribooligonucleotides, phosphate-sugar-backbone modified oligonucleotides, nucleotide analogs and mixtures thereof.

As used herein, the term “substituted acceptor probe,” “SAP”, “substituted acceptor polynucleotide probe” or “substituted acceptor oligonucleotide probe” refers to an acceptor probe with any thermolabile substitution group. In some embodiments, a SAP has more than one thermolabile substitution group. SAPs include those depicted herein, for example, Formula III.

As used herein, the term “donor,” “donor polynucleotide probe,” “donor oligonucleotide probe” “5′-phosphorylated donor polynucleotide probe,” “5′-phosphorylated donor oligonucleotide probe” or “donor probe” refers to a polynucleotide or oligonucleotide with a 5′ phosphate capable of being ligated to an acceptor probe. A donor probe may be suitable for ligation when hybridized in close proximity to an acceptor probe on a complementary target nucleic acid in conditions suitable for nucleic acid ligation; preferably donor and acceptor probes hybridize adjacent to each other on a complementary target nucleic acid. In some embodiments, a donor probe has at least one nucleic acid site that is not complementary (mismatch) to a target nucleic acid. In particular embodiments, the mismatch is at a nucleotide position of interest (e.g., SNP site). Additional alternative donor probes suitable for the methods and compositions provided herein include, but are not limited to, substituted, unsubstituted and modified nucleic acids, substituted, unsubstituted and modified ribonucleotides, substituted, unsubstituted and modified deoxyribonucleotides, substituted, unsubstituted and modified deoxyribooligonucleotides, substituted, unsubstituted and modified ribooligonucleotides, phosphate-sugar-backbone modified oligonucleotides, nucleotide analogs and mixtures thereof. In preferred embodiments, the donor probe is an oligonucleotide.

As used herein, the term “substituted donor probe”, “SDP”, “substituted donor polynucleotide probe,” “substituted donor oligonucleotide” or “substituted donor oligonucleotide probe” refers to a donor probe with thermolabile substitution group. In some embodiments, a SDP has more than one thermolabile substitution group. SDPs include those depicted herein, for example, Formula IV.

As used herein, the term “adenylated donor intermediate,” “adenylate-donor intermediate,” “adenylate-donor polynucleotide intermediate,” or “adenylate-donor oligonucleotide intermediate” refers to a polynucleotide or oligonucleotide with an adenylate residue attached by pyrophosphate linkage to 5′ phosphate of the donor intermediate and is capable of being ligated to an acceptor probe. An adenylate-donor intermediate may be suitable for ligation when hybridized in close proximity to an acceptor probe on a complementary target nucleic acid in conditions suitable for nucleic acid ligation; preferably an acceptor probe and adenylate-donor intermediate hybridize adjacent to each other on a complementary target nucleic acid. In some embodiments, adenylate-donor intermediate has at least one nucleic acid site that is not complementary (mismatch) to a target nucleic acid. In particular embodiments, the mismatch is at a nucleotide position of interest (e.g., SNP site). Additional alternative polynucleotide or oligonucleotide adenylate-donor intermediate suitable for the methods and compositions provided herein include, but are not limited to, substituted, unsubstituted and modified nucleic acids, substituted, unsubstituted and modified ribonucleotides, substituted, unsubstituted and modified deoxyribonucleotides, substituted, unsubstituted and modified deoxyribooligonucleotides, substituted, unsubstituted and modified ribooligonucleotides, phosphate-sugar-backbone modified oligonucleotides, nucleotide analogs and mixtures thereof. In preferred embodiments, the adenylate-donor intermediate is an oligonucleotide. As used herein, the term “substituted adenylate-donor intermediate”, “SADI”, “substituted adenylate-donor polynucleotide intermediate,” or “substituted adenylate-donor oligonucleotide intermediate” refers to an adenylated donor intermediate with a thermolabile substitution group. In some embodiments, a substituted adenylate-donor probe has more than one thermolabile substitution group on adenylate and/or donor oligonucleotide moieties. SADIs include those depicted herein, for example, Formula V.

As used herein, the term “adenylate,” “adenylate residue” or “adenylate moiety” refers to any AMP moiety, or equivalent thereof, that is transferred from ligase cofactor forming an activated or adenylated enzyme. The term “adenylate,” “adenylate residue” or “adenylate moiety” also refers to any AMP moiety, or equivalent thereof, that is transferred from an activated or adenylated enzyme to a 5′-phosphate of a donor probe. The adenylate can be substituted, unsubstituted and/or modified with a thermolabile group.

As used herein, the term “substitution group” refers to any chemical group or function that can be substituted for one or more atoms of a ligase component (e.g., replacement of a hydrogen with a substitutent such as alkyl, halo or the like, or replacement of one heteroatom of another (e.g., O for N)). In some embodiments, the chemical substitution group is attached enzymatically or chemically.

As used herein, the term “thermolabile substitution group,” “thermally labile group” or “thermolabile group” in relation to SLCs refers to a substitution group as disclosed herein, which is stable at ambient temperature but dissociate, cleave or otherwise is removed by incubating SLC at elevated temperature in a buffer which is compatible with enzymatic reaction. As a result of heat treatment the SLC transforms to a natural or unsubstituted ligase component, or equivalent of thereof, which fully supports ligase reaction. The thermolabile substitution group may be attached to SLC at any position, which includes but are not limited to the sugar, polyphosphate moiety, nucleoside base or internucleotide linkage. The thermolabile substitution group may be a group of any chemical nature, which makes SLC incompatible with the process of nucleic acid ligation, replication and amplification until this thermolabile substitution group is removed by heat. In preferred embodiments, the SLC containing thermolabile substitution group does not support or impedes or inhibits ligation, replication or amplification. In preferred embodiments, the thermolabile substitution group, when attached to ligase component results in SLC, which does not support, reduces, inhibits, impedes or eliminates formation of ligation product in matched and mismatched nucleic acid complexes as compared with ligation in the presence of unsubstituted ligase component.

As used herein, the term “thermostable irreversible substitution” or “thermostable substitution,” “thermostable substitution,” “thermostable group” and “thermostable substitution group” in relation to SLC refers to a chemical group of present invention which is stable and does not dissociates, cleaves or otherwise is removed by incubating SLC at elevated temperature in buffer which is compatible with enzymatic reaction. Thermostable substitution group may be attached to SLC at any position which includes but are not limited to the sugar, polyphosphate moiety, nucleoside base or internucleotide linkage.

As used herein, the term “ligase component” refers to ligase cofactor, acceptor probe, donor probe and/or adenylate-donor intermediate collectively, each individually, or combinations of any two or more thereof. For example, ligase component may refer to substituted or unsubstituted: ligase cofactor; ligase cofactor and donor probe; ligase cofactor and acceptor probe; or ligase cofactor, acceptor probe and donor probe; acceptor probe and adenylate-donor intermediate; or acceptor probe and donor probe.

As used herein, the term “substituted ligase component” (SLC) refers SLC, SAP, SDP or SADI, collectively to each individually, or combinations of any two or more thereof. For example, SLCs may refer to SC only; SC having one type of substitution; SC having more than one type of substitution; SC and SDP; SC and SAP; SC; SAP and SDP; SAP and SADI; SAP and SDP.

As used herein, the term “ligation” or “ligate” refers to methods for joining oligonucleotides and polynucleotide probes. Preferably ligation refers to joining the 3′-end of an acceptor probe to the 5′-end of a donor probe. In some embodiments, ligation refers to joining a nicked nucleic acid duplex. Typically, a nicked nucleic acid duplex consists of a 3′-hydroxyl acceptor oligonucleotide probe hybridized to a complementary nucleic acid template, with a 5′-phosphorylated donor oligonucleotide probe hybridized immediately 3′-downstream of an acceptor oligonucleotide probe. In some embodiments, a nicked nucleic acid duplex consists of a 3′-hydroxyl acceptor oligonucleotide probe hybridized to a complementary nucleic acid template, with 5′-adenylated donor oligonucleotide intermediate hybridized immediately 3′-downstream of an acceptor oligonucleotide probe. In some embodiments, a nick in duplex nucleic acid is ligated to form a phosphodiester linkage or equivalent internucleotide linkage, thereby forming a longer, complementary copy of the template nucleic acid sequence. Ligation involving the compositions and methods provided herein may employ one or more SC, one or more SAPs, one or more SDPs, one or more SADIs joining by nucleic acid ligase. Ligation of donor and acceptor probes or adenylated-donor intermediate and acceptor probe upon a target nucleic acid may occur with or without turnover of the probes. Preferably, ligation occurs with turnover, which is mediated by temperature cycling protocol such as LCR. A template nucleic acid may be deoxyribonucleic acid (DNA), ribonucleic acid (RNA), complementary DNA (cDNA), peptide nucleic acid (PNA), locked nucleic acid (LNA), hexitol nucleic acids (HNA), and/or any modified nucleic acid template. While the exemplary methods described hereinafter relate to ligation, numerous other methods suitable for the methods and compositions provided herein are known in the art for enzymatic ligation of nucleic acids. As used herein, the term “ligation junction” refers to a position on nucleic acid template where donor and acceptor probes ligate and form internucleotide linkage.

As used herein, the term “ligase” or “nucleic acid ligase” refers to an enzyme that is capable of template dependent and/or template independent ligation of nucleic acid. Preferably a ligase is capable of ligating the 5′-end of phosphorylated donor probe to the 3′-end of an acceptor probe in the presence of ligase cofactor, or is capable of ligating the 5′-phosphate of adenylated donor intermediate to the 3′-end of an acceptor probe without ligase cofactor. In other embodiments, the ligation may involve DNA, RNA, cDNA, PNA, LNA, HNA, and/or other modified nucleic acids. In some embodiments, the ligase is one of the following: bacteriophage T4 DNA ligase, E. coli DNA ligase, Aquifex aeolicus DNA ligase, Taq DNA ligase, 9° N™ DNA ligase, Methanobacterium thermoautotrophicum RNA ligase, Ferroplasma acidiphilum DNA ligase, Human DNA ligase I, Human DNA ligase II, Human DNA ligase III, Human DNA ligase IV, Vaccinia virus DNA ligase, Chlorella virus DNA ligase, Pyrococcus furiosis DNA ligase, Haloferax volcanii DNA ligase, Acidianus ambivalens DNA ligase, Archaeoglobus fulgidus DNA ligase, Aeropyrum pernix DNA ligase, Cenarcheon symbiosum DNA ligase, Haloarcula marismortui DNA ligase, Ferroplasma acidarmanus DNA ligase, Natronomonas pharaosis DNA ligase, Haloquadratum walsbyi DNA ligase, Halobacterium salinarum DNA ligase, Methanosarcina acetivorans DNA ligase, Methanosarcina barkeri DNA ligase, Methanococcoides burtonii DNA ligase, Methanospirillum hungatei DNA ligase, Methanocaldococcus jannaschii DNA ligase, Methanopyrus kandleri DNA ligase, Methanosarcina mazei DNA ligase, Methanococcus maripaludis DNA ligase, Methanosaeta thermophila DNA ligase, Methanosphaera stadtmanae DNA ligase, Methanothermobacter thermautotrophicus DNA ligase, Nanoarchaeum equitans DNA ligase, Pyrococcus abyssi DNA ligase, Pyrobaculum aerophilum DNA ligase, Pyrococcus horikoshii DNA ligase, Picrophilus torridus DNA ligase, Sulfolobus acidocaldarius DNA ligase, Sulfolobus shibatae DNA ligase, Sulfolobus solfataricus DNA ligase, Sulfolobus tokodaii DNA ligase, Thermoplasma acidophilum DNA ligase, Thermococcus fumicolans DNA ligase, Thermococcus kodakarensis DNA ligase, Thermococcus sp. NA1 DNA ligase, Thermoplasma volcanium DNA ligase, Staphylococcus aureus DNA ligase, Thermus scotoductus NAD⁺-DNA ligase, T4 RNA ligase, Staphylococcus aureus DNA ligase, Methanobacterium thermoautotrophicum DNA ligase, Thermus aquaticus DNA ligase, Thermus species AK16D DNA ligase, Haemophilus influenzae DNA ligase, Thermus thermophilus DNA ligase, bacteriophage T7 DNA ligase, Haemophilus influenzae DNA ligase, Mycobacterium tuberculosis DNA ligase, Deinococcus radiodurans RNA ligase, Methanobacterium thermoautotrophicum RNA ligase, Rhodothermus marinus RNA ligase, Trypanosoma brucei RNA ligase, marine archaea Thermococcus sp. (strain 9° N) DNA ligase, bacteriophage T4 RNA ligase 1, Ampligase, or bacteriophage T4 RNA ligase 2.

As used herein, the term “monitoring ligation” refers to detecting the presence, detecting the absence and/or measuring the amount of ligated nucleic acid. Ligation may be monitored, for example, by detecting and/or quantifying the amount of ligation product using a detectable label or by correlating the presence and/or amount of a product of a subsequent process to the presence and/or amount of ligation product (e.g., by directly correlating the presence and/or amount of subsequent amplification of ligated products to the amount of ligation product). Monitoring ligation also includes any method of assessing the size of nucleic acids to indicate whether ligation has occurred or not or to assess what portion of total nucleic acid present in a sample has ligated and what portion has not; such results may be expressed in terms of a percentage or a ratio. Monitoring ligation includes any of the methods disclosed herein (e.g., gel electrophoresis) as well as methods known in the art.

As used herein, the term “ligase mediated replication,” “replication” or “replicate” in respect to ligase reaction refers to one or more methods for copying a target nucleic acid, thereby increasing the number of copies of a selected nucleic acid sequence when at least one step of the process involved in replication of nucleic acid uses ligase or other enzyme capable of ligating nucleic acid. Replication of the present invention employs natural and/or synthetic oligo- or polynucleotide probes, natural and/or synthetic adenylated donor intermediate, natural and/or synthetic cofactor and natural and/or artificial nucleic acid ligase. Replication is equivalent to amplification when multiple copies of target nucleic acid sequence are generated. Replication may be exponential or linear. A target nucleic acid may be DNA, RNA, cDNA or any modified nucleic acid template. While the exemplary methods described hereinafter relate to replication using ligase reaction and ligase chain reaction (LCR), numerous other methods are known in the art for replication of nucleic acids. For example methods include isothermal methods, rolling circle methods, Allele-specific LCR, Assembly LCR or Ligase Cycling Assembly (LCA), Asymmetric LCR, Colony LCR, Emulsion LCR, Fast LCR, Gap Extension Ligation PCR (GEXL-PCR), Gap Ligation Chain Reaction (Gap LCR), Hot Start LCR, Ligation-mediated PCR, Linear-After-The-Exponential-LCR (LATE-LCR), Methylation-specific LCR (MSL), Multiplex Ligation-dependent Probe Amplification, (MLPA), Multiplex LCR, Nested LCR, Quantitative LCR (Q-LCR), Quantitative real-time LCR (QRT-LCR), Real-Time LCR, Hot Start real-Time LCR, Reverse Transcription LCR (RT LCR), Single molecule amplification LCR(SMA LCR), Touchdown LCR, nucleic acid ligation, ligase mediated DNA sequencing, OLA, LDR, and ligase mediated PCR, MLPA, DNA sequencing and other applications that involve nucleic acid ligase reaction. The skilled artisan will understand that other methods may be used either in place of, or together with ligation and LCR methods.

As used herein, the term “amplification” or “amplify” refers to one or more methods known in the art for copying a target nucleic acid, thereby increasing the number of copies of a selected nucleic acid sequence. Amplification of the present invention employs natural and/or synthetic oligo- or polynucleotide nucleotide probes, adenylated donor intermediate, ligase cofactor, natural and/or artificial nucleic acid ligase and may include other enzymes such as nucleic acid polymerase and/or reverse transcriptase (RT). Amplification may be exponential or linear. A target nucleic acid may be DNA, RNA, cDNA or any modified nucleic acid template. While the exemplary methods described hereinafter relate to amplification using the LCR, numerous other methods are known in the art for amplification of nucleic acids. For example methods include isothermal methods, rolling circle methods, ligase mediated PCR, MLPA, GEXL PCR, GAP LCR, real-time LCR, quantitative LCR, multiplex LCR, DNA sequencing and other applications that involve nucleic acid ligase reaction. The skilled artisan will understand that other methods may be used either in place of, or together with ligation and LCR methods. See, Wiedmann, et al., Genome Res. 3, S51-S64 (1994); Barany, Proc Natl Acad Sci USA 88, 189-193 (1991).

As used herein, the term “nucleic acid” refers to a polynucleotide, an oligonucleotide, or any fragment thereof, any ribo or deoxyribo derivatives and to naturally occurring or synthetic molecules containing natural and/or modified nucleotide residues and internucleotide linkages. These term also refer to DNA or RNA of natural (e.g., genomic) or synthetic origin which may be single-stranded, double-stranded, triple-stranded or tetra-stranded and may represent the sense or the antisense strand, or to any DNA-like or RNA-like material. An “RNA equivalent,” in reference to a DNA sequence, is composed of the same linear sequence of nucleotides as the reference DNA sequence with the exception that all or most occurrences of the nitrogenous base thymine are replaced with uracil, and the sugar backbone is composed of ribose instead of 2′-deoxyribose. Additional alternative nucleic acid backbones suitable for the methods and compositions provided herein include but are not limited to phosphorothioate, phosphoroselenoate, alkyl phosphotriester, aryl phosphotriester, alkyl phosphonate, aryl phosphonate, Locked Nucleic Acids (LNA), and Peptide Nucleic Acids (PNA) and phosphoboronate. RNA may be used in the methods described herein and/or may be converted to cDNA by reverse-transcription for use in the methods described herein.

As used herein, the term “polynucleotide” refers to a nucleic acid chain, usually single stranded, which may be naturally occurring or synthetic and may contain up to millions of nucleotides. Throughout this application, nucleic acids are designated by the 5′-terminus to the 3′-terminus Standard nucleic acids, e.g., DNA and RNA, are typically synthesized chemically in 3′ to 5′ direction by the addition of nucleotides to the 5′-terminus of a growing nucleic acid or in reverse 5′ to 3′ direction by the addition of nucleotides to the 3′-terminus of a growing nucleic acid. Otherwise, standard nucleic acids, e.g., DNA and RNA, are synthesized enzymatically by the addition of nucleotides to the 3′-terminus of a growing nucleic acid. Polynucleotides may be DNA, RNA, PNA, LNA, HNA and may include other modified nucleosides, or combinations of any two or more thereof. In some embodiments, a polynucleotide is an oligonucleotide. As used herein, the term “nucleotide” refers to a subunit of a nucleic acid consisting of a phosphate group, a 5-carbon sugar and a nitrogenous base. The 5-carbon sugar found in RNA is ribose. In DNA, the 5-carbon sugar is 2′-deoxyribose. The term “polynucleotide” also includes analogs of such subunits.

As used herein, the term “oligonucleotide” refers to a polynucleotide having a sequence of between 2 to about 70 nucleotides, more preferably about 5 to about 50 nucleotides, more preferably about 10 to about 30 nucleotides or more preferably about 15 to about 25 nucleotides. In some embodiments, an oligonucleotide includes a sequence of at least 5 nucleotides, at least 10 nucleotides, at least 15 nucleotides, at least 20 nucleotides, at least 25 nucleotides, at least 30 nucleotides, at least 35 nucleotides, at least 40 nucleotides, at least 50 nucleotides, at least 55 nucleotides or at least 60 nucleotides in length; or less than 70 nucleotides, less than 65 nucleotides, less than 60 nucleotides, less than 55 nucleotides, less than 50 nucleotides, less than 45 nucleotides, less than 40 nucleotides, less than 35 nucleotides, less than 30 nucleotides, less than 25 nucleotides, less than 20 nucleotides, less than 15 nucleotides; less than 10 nucleotides, less than 5 nucleotides, or combinations of any two or more thereof, in length.

As used herein, the term “probe” or “oligonucleotide probe” in relation to ligase reaction refers to a polynucleotide or oligonucleotide suitable for a ligase mediated replication. The skilled artisan is capable of designing and preparing probes that are appropriate for replication of a target sequence. The length of probes for use in the methods and compositions provided herein depends on several factors including the nucleotide sequence identity and the temperature at which these nucleic acids are hybridized or used during in vitro nucleic acid replication, ligation or amplification. The considerations necessary to determine a preferred length for the probe of a particular sequence identity are well known to the person of ordinary skill. For example, the length of a short nucleic acid or oligonucleotide can relate to its hybridization specificity or selectivity. As used herein, the term “probe binding sequence” or “PBS” refers to a nucleic acid region that specifically hybridizes or anneals to a specified probe.

As used herein, the term “detection probe” also refers to a polynucleotide or oligonucleotide suitable for detecting the presence or absence of specified nucleic acid.

As used herein, the term “target nucleic acid” refers to any nucleic acid of interest.

As used herein, the term “template nucleic acid” refers to a nucleic acid capable of binding to a donor and/or acceptor probes. Preferably the template nucleic acid includes a target nucleic acid.

As used herein, the term “mismatch” refers to a nucleoside residue in oligonucleotide or polynucleotide probe that is not complementary to a respective nucleoside residue in nucleic acid target sequence. As used herein, the term “mismatch template” or “mismatched template” refers to a nucleic acid sequence where at least one nucleoside residue on the template strand is not complementary with respective nucleoside residue, or paired with an incorrect nucleoside of oligonucleotide or polynucleotide probe. Complementary nucleosides are A and T (or U) and C and G. A ligation reaction on mismatched template occurs typically with less than 100% fidelity/specificity. As used herein, the term “matched template” refers to a target nucleic acid sequence where all bases are complementary to oligonucleotide probe or probes.

As used herein, the term “single nucleotide polymorphism” or “SNP” refers to a single base genetic sequence variation between different individuals of a species or other specified population. In some embodiments, SNPs are single base pair positions at a specified nucleic acid site in genomic DNA at which different sequence alternatives (alleles) exist in normal individuals in some population(s) where the least frequent allele has an abundance of 1% or greater; or 0.8% or greater; or 0.5% or greater; or 0.4% or greater; or 0.3% or greater; or 0.2% or greater; or 0.1% or greater. In some embodiments, a SNP of interest is known by one of ordinary skill in the art, for example, a particular SNP is published in a scientific journal such as those accessible through Pubmed (available on the world wide web at ncbi.nlm.nih.gov/pubmed/) such as Science, Nature, PNAS and NEJM. In some embodiments, a SNP can be found in a database of polymorphisms such as those found at Entrez SNP (available on the world wide web at ncbi.nlm.nih.gov/sites/entrez?db=snp) or a human SNP database (available on the world wide web at ncbi.nlm.nih.gov/projects/SNP/). In some embodiments, a population includes all humans as a whole or a subset of humans, such as a group of people of a particular race, nationality, geographical region, family lineage, gender, age, or from a particular period of time or era.

As used herein, the term “single nucleotide polymorphism site,” “SNP site,” or “SNP position” refers to a nucleic acid position where a SNP is known to occur.

As used herein, the term “terminus” with respect to an oligonucleotide or polynucleotide refers to the last nucleotides at the 3′ or 5′ end of polynucleotide or oligonucleotide. Preferably the terminus of oligo- or polynucleotide includes the terminal 6 nucleotides, more preferably the terminal 5 nucleotides, more preferably the terminal 4 nucleotides, more preferably the terminal 3 nucleotides, more preferably the terminal 2 nucleotides, or more preferably the very terminal nucleotide.

As used herein, the term “label” or “detectable label” refers to any compound or combination of compounds that may be attached or otherwise associated with a molecule so that the molecule can be detected directly or indirectly by detecting the label. A detectable label can be a radioisotope (e.g., carbon, phosphorus, iodine, indium, sulfur, tritium etc.), a mass isotope (e.g., H², C¹³ or N¹⁵), a dye or fluorophore (e.g., cyanine, fluorescein or rhodamine), a hapten (e.g., biotin) or any other agent that can be detected directly or indirectly. After incorporation of a labeled nucleotide into a nucleic acid the label may be detected.

As used herein, the term “hybridize” or “specifically hybridize” refers to a process where two or more complementary nucleic acid strands anneal to each other under appropriately stringent conditions. Hybridizations to target nucleic acids are typically and preferably conducted with oligonucleotide probe molecules, preferably 10-100 nucleotides in length. Nucleic acid hybridization techniques are well known in the art. See, e.g., Sambrook, et al., Molecular Cloning: A Laboratory Manual, Second Edition, Cold Spring Harbor Press, Plainview, N.Y. (1989); Ausubel, F. M., et al., Current Protocols in Molecular Biology, John Wiley & Sons, Secaucus, N.J. (1994).

As used herein, the term “stringent hybridization condition,” “high stringency hybridization condition” or “stringent conditions” refers to hybridization conditions which do not allow for hybridization of two nucleic acids sequences which are not completely complementary. As used herein, the term “non-stringent hybridization condition” or “low stringency hybridization condition” or “low stringent conditions” refers to hybridization conditions which allow for hybridization of two nucleic acids sequences which are not completely complementary or contain mismatch nucleotides at one or more position.

As used herein, the term “sample” or “test sample” refers to any liquid or solid material believed to include nucleic acid of interest. A test sample may be obtained from any biological source (i.e., a biological sample), such as cells in culture or a tissue sample or synthetically produced including a chemically synthesized template.

As used herein, the term “complement,” “complementary,” or “complementarity” in the context of an oligonucleotide or polynucleotide (i.e., a sequence of nucleotides such as an oligonucleotide probe or a target nucleic acid) refers to standard Watson/Crick base pairing rules. A complement sequence can also be a sequence of DNA or RNA complementary to the DNA sequence or its complement sequence, and can also be a cDNA. For example, the sequence “5′ A G T C 3′” is complementary to the sequence “3′ T C A G 5′.” Certain nucleotides not commonly found in natural nucleic acids or chemically synthesized comply with complementary rules and may be included in the nucleic acids described herein; these include but are not limited to base and sugar modified nucleosides and nucleotides, such as inosine, 7-deazaguanosine, 8-aza-7-deazaguanosine 2′-O-methylguanosine, 2′-fluoro-2′-deoxycytidine, Locked nucleosides and nucleotides (LNA), and components of Peptide Nucleic Acids (PNA). Those skilled in the art of nucleic acid technology can determine duplex stability empirically considering a number of variables including, for example, the length of the oligonucleotide, base composition and sequence of the oligonucleotide, incidence of mismatched base pairs, ionic strength, other hybridization buffer components and conditions.

Complementarity may be complete or may be partial in which only some of the nucleotide bases of two nucleic acid strands are matched according to the base pairing rules. Complementarity may be complete or perfect or total where all of the nucleotide bases of two nucleic acid strands are matched according to the base pairing rules. Complementarity may be incomplete or imperfect where duplexes may contain mismatched base pairs, degenerative, missing or unmatched nucleotides. Complementarity may be absent where none of the nucleotide bases of two nucleic acid strands are matched according to the base pairing rules. The degree of complementarity between nucleic acid strands has significant effects on the efficiency and strength of hybridization between nucleic acid strands. This is of particular importance in replication, ligation and amplification reactions, as well as detection methods that depend upon binding between nucleic acids. The terms may be used in reference to individual nucleotides, especially within the context of nucleic acid duplexes. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand in the duplex.

As used herein, the term “substantially complementary” refers to two sequences that hybridize under non-stringent hybridization conditions. The skilled artisan will understand that substantially complementary sequences need not hybridize along their entire length. In particular, substantially complementary sequences may include a contiguous sequence of nucleotides that do not hybridize to a target sequence, positioned 3′ or 5′ to a contiguous sequence of nucleotides that hybridize under stringent hybridization conditions to a target sequence.

As used herein, a polynucleotide probe or an oligonucleotide probe is “perfectly specific” for a nucleic acid sequence if the polynucleotide or oligonucleotide probe hybridization sequence of the polynucleotide probe or oligonucleotide probe has 100% sequence identity with a target nucleic acid sequence when the polynucleotide probe or oligonucleotide probe and the nucleic acid sequences are aligned. A polynucleotide probe or oligonucleotide probe is “specific” for a nucleic acid sequence if under the appropriate hybridization or washing conditions, is capable of hybridizing to the target of interest and not substantially hybridizing to nucleic acids sequences which are not of interest. Higher levels of sequence identity are preferred and include at least 75%, at least 80%, at least 85%, at least 90%, at least 95%, at least 99%, and more preferably 100% sequence identity.

As used herein, the term “nucleoside” includes all naturally occurring nucleosides, including all forms of nucleoside bases and furanosides found in natural nucleic acids. Base rings most commonly found in naturally occurring nucleosides are purine and pyrimidine rings. Naturally occurring purine rings include, for example, adenine, guanine, and N⁶-methyladenine. Naturally occurring pyrimidine rings include, for example, cytosine, thymine, uracyl and 5-methylcytosine. Naturally occurring nucleosides for example include but not limited to ribo and 2′-deoxyribo derivatives of adenosine, guanosine, cytidine, thymidine, uridine, inosine, 7-deazaguanosine, and 7-methylguanosine.

As used herein, the terms “nucleoside analogs,” “modified nucleosides,” or “nucleoside derivatives” include synthetic unnatural nucleosides as described herein. Nucleoside analogs and derivatives include nucleosides having modified base or/and sugar moieties or containing protecting groups. Such analogs include, for example, 2′-deoxy-2′-fluorouridine, 3′-O-methyluridine and the like. The compounds and methods provided herein include such synthetic analogs thereof, as well as heterocycle-substituted sugars, and even acyclic substituted bases. Moreover, nucleoside derivatives include other purine and pyrimidine analogs, for example, halogen-substituted purines (e.g., 6-fluoropurine), halogen-substituted pyrimidines (e.g. 5-iodouracyl), N⁶-ethyladenine, N⁴-(alkyl)-cytosines, 5-ethylcytosine, and the like. Nucleoside derivatives and analogs encompass a wide variety of substitutions, such as those described in U.S. Pat. No. 6,762,298.

As used herein, the terms “universal base nucleoside,” “degenerate base nucleoside,” “universal base nucleoside analog” and “degenerate base nucleoside analog” include, for example, a nucleoside analog with an artificial base which is preferably recognizable by nucleic acid enzyme as a substitute for any natural nucleoside such as adenosine, guanosine, cytidine, thymidine, uridine and other. For example, nucleoside 5′-triphosphates with universal bases or degenerate bases can be found in Loakes, D., Nucleic Acids Res. 29, 2437-2447 (2001); Crey-Desbiolles, C., et al., 33 Nucleic Acids Res. 1532-1543 (2005); Kincaid, K., et al., 33 Nucleic Acids Res. 2620-2628 (2005); Preparata, F P, Oliver, J S, 11 J. Comput. Biol. 753-765 (2004); and Hill, F., et al., Proc. Natl. Acad. Sci. USA 95, 4258-4263 (1998).

As used herein, the term “internucleotide linkage” refers to the bond or bonds that connect two nucleosides of an oligonucleotide probe or nucleic acid and may be a natural phosphodiester linkage or substituted internucleotide linkage.

As used herein, the term “hydrocarbyl” refers to any organic radical where the backbone thereof comprises carbon and hydrogen only. Thus, hydrocarbyl embraces alkyl, cycloalkyl, alkenyl, cycloalkenyl, alkynyl, aryl, alkylaryl, arylalkyl, arylalkenyl, alkenylaryl, arylalkynyl, alkynylaryl, and the like.

As used herein, the term “substituted hydrocarbyl” refers to any of the above-referenced hydrocarbyl groups further bearing one or more substituents selected from hydroxy, hydrocarbyloxy, substituted hydrocarbyloxy, alkylthio, substituted alkylthio, arylthio, substituted arylthio, amino, alkylamino, substituted alkylamino, carboxy, —C(S)SR, —C(O)SR, —C(S)NR₂, where each R is independently hydrogen, alkyl or substituted alkyl, nitro, cyano, halo, —SO₃M or —OSO₃M, where M is H, Na, K, Zn, Ca, or meglumine, guanidinyl, substituted guanidinyl, hydrocarbyl, substituted hydrocarbyl, hydrocarbylcarbonyl, substituted hydrocarbylcarbonyl, hydrocarbyloxycarbonyl, substituted hydrocarbyloxycarbonyl, hydrocarbylcarbonyloxy, substituted hydrocarbylcarbonyloxy, acyl, acyloxy, heterocyclic, substituted heterocyclic, heteroaryl, substituted heteroaryl, heteroarylcarbonyl, substituted heteroarylcarbonyl, carbamoyl, monoalkylcarbamoyl, dialkylcarbamoyl, arylcarbamoyl, a carbamate group, a dithiocarbamate group, aroyl, substituted aroyl, organosulfonyl, substituted organosulfonyl, organosulfinyl, substituted alkylsulfinyl, alkylsulfonylamino, substituted alkylsulfonylamino, arylsulfonylamino, substituted arylsulfonylamino, a sulfonamide group, sulfuryl, and the like, including two or more of the above-described groups attached to the hydrocarbyl moiety by such linker/spacer moieties as —O—, —S—, —NR—, where R is hydrogen, alkyl or substituted alkyl, —C(O)—, —C(S)—, —C(═NR′)—, —C(═CR′₂)—, where R′ is alkyl or substituted alkyl, —O—C(O)—, —O—C(O)—O—, —O—C(O)—NR— (or —NR—C(O)—O—), —NR—C(O)—, —NR—C(O)—NR—, —S—C(O)—, —S—C(O)—O—, —S—C(O)—NR—, —O—S(O)₂—, —O—S(O)₂—O—, —O—S(O)₂—NR—, —O—S(O)—, —O—S(O)—O—, —O—S(O)—NR—, —O—NR—C(O)—, —O—NR—C(O)—O—, —O—NR—C(O)—NR—, —NR—O—C(O)—, —NR—O—C(O)—O—, —NR—O—C(O)—NR—, —O—NR—C(S)—, —O—NR—C(S)—O—, —O—NR—C(S)—NR—, —NR—O—C(S)—, —NR—O—C(S)—O—, —NR—O—C(S)—NR—, —O—C(S)—, —O—C(S)—O—, —O—C(S)—NR— (or —NR—C(S)—O—), —NR—C(S)—, —NR—C(S)—NR—, —S—S(O)₂—, —S—S(O)₂—O—, —S—S(O)₂—NR—, —NR—O—S(O)—, —NR—O—S(O)—O—, —NR—O—S(O)—NR—, —NR—O—S(O)₂—, —NR—O—S(O)₂—O—, —NR—O—S(O)₂—NR—, —O—NR—S(O)—, —O—NR—S(O)—O—, —O—NR—S(O)—NR—, —O—NR—S(O)₂—O—, —O—NR—S(O)₂—NR—, —O—NR—S(O)₂—, —O—P(O)R₂—, —S—P(O)R₂—, or —NR—P(O)R₂—, where each R is independently hydrogen, alkyl or substituted alkyl, and the like.

As used herein, the term “alkane” refers to an organic compound that includes carbon atoms and hydrogen atoms, and includes C—H bonds and additionally includes C—C single bonds in alkanes other than methane. The term “alkane” includes straight-chain alkanes such as alkanes having from 1 to 20 carbon atoms. In some embodiments, alkanes include straight-chain alkanes such as alkanes having from 1 to 8 carbon atoms such as methane, ethane, propane, butane, pentane, hexane, heptane, and octane. The term “alkane” also includes branched-chain alkanes such as, but not limited to branched chain alkanes having from 1 to 20, and in some embodiments from 1 to 8 carbon atoms such as, but not limited to, 2-methylpropane, 2,2-dimethylpropane, 2-methylbutane, 2,3-dimethylbutane, 2,2-dimethylbutane, 2-methylpentane, 3-methylpentane, 2,3-dimethylpentane, 2,4-dimethylpentane, 2,2-dimethylpentane, 3,3-dimethylpentane, 2-methylhexane, 3-methylhexane, 2,2-dimethylhexane, 2,3-dimethylhexane, 2,4-dimethylhexane, 2,5-dimethylhexane, 3,3-dimethylhexane, 3,4-dimethylhexane, 2-methylheptane, 3-methylheptane, 4-methylheptane, 3-ethylpentane, 3-ethyl-2-methylpentane, 3-ethylhexane, and the like. A C—C or a C—H bond of an alkane may be replaced with a bond to another group such as a hydroxyl group, a halogen such as F, Cl, Br, or I, a sulfhydryl group, or an amine group. Alkanes replaced with such groups may respectively be named as hydroxyalkanes, haloalkanes such as fluoroalkanes, chloroalkanes, bromoalkanes, iodoalkanes, mercaptoalkanes, and aminoalkanes.

As used herein, the term “alkyl” refers to a single bond chain of hydrocarbons usually ranging from 1-20 carbon atoms, preferably 1-8 carbon atoms, examples include methyl, ethyl, propyl, isopropyl, and the like. Examples of such alkyl radicals include methyl, ethyl, propyl, isopropyl, n-butyl, sec-butyl, isobutyl, tert-butyl, pentyl, isoamyl, hexyl, octyl, dodecanyl, and the like.

As used herein, the term “lower alkyl” refers to a straight chain or a branched chain of hydrocarbons usually ranging from 1-6 carbon atoms, preferably 2-5 carbon atoms. Examples include ethyl, propyl, isopropyl, and the like.

As used herein, the term “hydrocarbylene” refers to any divalent organic radical wherein the backbone thereof comprises carbon and hydrogen only. Thus, hydrocarbylene embraces alkylene, cycloalkylene, alkenylene, cycloalkenylene, alkynylene, arylene, alkylarylene, arylalkylene, arylalkenylene, alkenylarylene, arylalkynylene, alkynylarylene, and the like, and “substituted hydrocarbylene” refers to any of the above-referenced hydrocarbylene groups further bearing one or more substituents as set forth herein.

As used herein, the term “alkylene” refers to a divalent hydrocarbyl containing 1-20 carbon atoms, preferably 1-15 carbon atoms, straight chain or branched, from which two hydrogen atoms are taken from the same carbon atom or from different carbon atoms. Examples of alkylene include, but are not limited to, methylene (—CH₂—), ethylene (—CH₂CH₂—), and the like.

As used herein, the term “alkynyl” refers to a straight-chain or branched-chain hydrocarbyl, which has one or more triple bonds and contains from about 2-20 carbon atoms, preferably from about 2-10 carbon atoms, more preferably from about 2-8 carbon atoms, and most preferably from about 2-6 carbon atoms. Examples of alkynyl radicals include ethynyl, propynyl (propargyl), butynyl, and the like.

As used herein, the term “alkynylaryl” refers to alkynyl-substituted aryl groups and “substituted alkynylaryl” refers to alkynylaryl groups further bearing one or more substituents as set forth herein.

As used herein, the term “hydrocarbyloxy” denotes —O-hydrocarbyl groups containing 2-20 carbon atoms and “substituted hydrocarbyloxy” refers to hydrocarbyloxy groups further bearing one or more substituents as set forth herein.

As used herein, the term “alkoxy” denotes the group —OR^{c}, where R^{c} is lower alkyl, substituted lower alkyl, aryl, substituted aryl, aralkyl, substituted aralkyl, heteroalkyl, heteroarylalkyl, cycloalkyl, substituted cycloalkyl, cycloheteroalkyl, or substituted cycloheteroalkyl as defined.

As used herein, the term “lower alkoxy” denotes the group —OR^{d}, where R^{d} is lower alkyl.

As used herein, the term “acyl” denotes the group —C(O)R^{a}, where R^{a} is hydrogen, lower alkyl, cycloalkyl, heterocyclyl, aryl, heteroaryl, and the like.

As used herein, the term “substituted acyl” denotes the group —C(O)R^{a′}, where R^{a′} is substituted lower alkyl, substituted cycloalkyl, substituted heterocyclyl, substituted aryl, substituted heteroaryl, and the like.

As used herein, the term “acyloxy” denotes the group —OC(O)R^{b}, where R^{b} is hydrogen, lower alkyl, substituted lower alkyl, cycloalkyl, substituted cycloalkyl, heterocyclyl, substituted heterocyclyl, aryl, substituted aryl, heteroaryl, substituted heteroaryl, and the like.

As used herein, the term “alkenyl” refers to a straight-chain or branched-chain hydrocarbyl, which has one or more double bonds and, unless otherwise specified, contains from about 2 to about 20 carbon atoms, preferably from about 2 to about 10 carbon atoms, more preferably from about 2 to about 8 carbon atoms, and most preferably from about 2 to about 6 carbon atoms. Examples of alkenyl radicals include vinyl, allyl, 1,4-butadienyl, isopropenyl, and the like.

As used herein, the term “alkenylaryl” refers to alkenyl-substituted aryl groups and “substituted alkenylaryl” refers to alkenylaryl groups further bearing one or more substituents as set forth herein.

As used herein, the term “alkenylene” refers to divalent straight or branched chain hydrocarbyl groups having at least one carbon-carbon double bond, and typically containing 2-20 carbon atoms, preferably 2-12 carbon atoms, preferably 2-8 carbon atoms, and “substituted alkenylene” refers to alkenylene groups further bearing one or more substituents as set forth herein.

As used herein, the term “alkylaryl” refers to alkyl-substituted aryl groups and “substituted alkylaryl” refers to alkylaryl groups further bearing one or more substituents as set forth herein.

As used herein, the term “alkylcarbonylamino” denotes the group —NR^{e}C(O)R^{f}, where R^{e} is optionally substituted alkyl, and R^{f} is hydrogen or alkyl.

As used herein, the term “alkylsulfinyl” denotes the group —S(O)R^{g}, where R^{g} is optionally substituted alkyl.

As used herein, the term “alkylsulfonyl” denotes the group —S(O)₂R^{g}, where R^{g} is optionally substituted alkyl.

As used herein, the term “alkylsulfonylamino” denotes the group —NR^{e}S(O)₂R^{f}, where R^{e} is optionally substituted alkyl, and R^{f} is hydrogen or alkyl.

As used herein, the term “alkylthio” refers to the group —S—R^{h}, where R^{h} is alkyl.

As used herein, the term “substituted alkylthio” refers to the group —S—Rⁱ, where Rⁱ is substituted alkyl.

As used herein, the term “alkynylene” refers to divalent straight or branched chain hydrocarbyl groups having at least one carbon-carbon triple bond, and typically having in the range of about 2-12 carbon atoms, preferably about 2-8 carbon atoms, and “substituted alkynylene” refers to alkynylene groups further bearing one or more substituents as set forth herein.

As used herein, the term “amido” denotes the group —C(O)NR^{j}R^{j′}, where R^{j} and R^{j′} may independently be hydrogen, lower alkyl, substituted lower alkyl, alkyl, substituted alkyl, aryl, substituted aryl, heteroaryl, or substituted heteroaryl.

As used herein, the term “substituted amido” denotes the group —C(O)NR^{k}R^{k′}, where R^{k} and R^{k′} are independently hydrogen, lower alkyl, substituted lower alkyl, aryl, substituted aryl, heteroaryl, or substituted heteroaryl, provided, however, that at least one of R^{k} and R^{k′} is not hydrogen. R^{k}R^{k′} in combination with the nitrogen may form an optionally substituted heterocyclic or heteroaryl ring.

As used herein, the term “amidino” denotes the group —C(═NR^{m})NR^{m′}R^{m″}, where R^{m}, R^{m′}, and R^{m″} are independently hydrogen or optionally substituted alkyl, aryl, or heteroaryl.

As used herein, the term “amino” or “amine” denotes the group —NRⁿR^{n′}, where Rⁿ and R^{n′} may independently be hydrogen, lower alkyl, substituted lower alkyl, alkyl, substituted alkyl, aryl, substituted aryl, heteroaryl, or substituted heteroaryl as defined herein. A “divalent amine” denotes the group —NH—. A “substituted divalent amine” denotes the group —NR— where R is lower alkyl, substituted lower alkyl, alkyl, substituted alkyl, aryl, substituted aryl, heteroaryl, or substituted heteroaryl.

As used herein, the term “substituted amino” or “substituted amine” denotes the group —NR^{p}R^{p′}, where R^{p} and R^{p′} are independently hydrogen, lower alkyl, substituted lower alkyl, alkyl, substituted alkyl, aryl, substituted aryl, heteroaryl, substituted heteroaryl, provided, however, that at least one of R^{p} and R^{p′} is not hydrogen. R^{p}R^{p′} in combination with the nitrogen may form an optionally substituted heterocyclic, or heteroaryl ring.

As used herein, the term “arylalkynyl” refers to aryl-substituted alkynyl groups and “substituted arylalkynyl” refers to arylalkynyl groups further bearing one or more substituents as set forth herein.

As used herein, the term “aralkyl” refers to alkyl as defined herein, where an alkyl hydrogen atom is replaced by an aryl as defined herein. Examples of aralkyl radicals include benzyl, phenethyl, 1-phenylpropyl, 2-phenylpropyl, 3-phenylpropyl, 1-naphthylpropyl, 2-naphthylpropyl, 3-naphthylpropyl, 3-naphthylbutyl, and the like.

As used herein, the term “aroyl” refers to aryl-carbonyl species such as benzoyl and “substituted aroyl” refers to aroyl groups further bearing one or more substituents as set forth herein.

As used herein, the term “arylalkyl” refers to aryl-substituted alkyl groups and “substituted arylalkyl” refers to arylalkyl groups further bearing one or more substituents as set forth herein.

As used herein, the term “aryl” alone or in combination refers to phenyl, naphthyl or fused aromatic heterocyclic optionally with a cycloalkyl of preferably 5-7, more preferably 5-6, ring members and/or optionally substituted with 1 to 3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like.

As used herein, the term “arylcarbonylamino” denotes the group —NR^{q}C(O)R^{r}, wherein R^{q} is hydrogen or lower alkyl or alkyl and R^{r} is optionally substituted aryl.

As used herein, the term “arylene” refers to divalent aromatic groups typically having in the range of 6 up to 14 carbon atoms and “substituted arylene” refers to arylene groups further bearing one or more substituents as set forth herein.

As used herein, the term “aryloxy” denotes the group —OAr, where Ar is an aryl, or substituted aryl group.

As used herein, the term “arylsulfonylamino” denotes the group —NR^{q}S(O)₂R^{r}, where R^{q} is hydrogen or lower alkyl, or alkyl and R^{r} is optionally substituted aryl.

As used herein, the term “a carbamate group” denotes the group —O—C(O)—NR₂, where each R is independently H, alkyl, substituted alkyl, aryl, or substituted aryl as set forth herein.

As used herein, the term “alkoxycarbonyl” denotes the group —C(O)—OR, where each R is independently H, alkyl, substituted alkyl, aryl, or substituted aryl as set forth herein.

As used herein, the term “dithiocarbamate group” denotes the group —S—C(S)—NR₂, where each R is independently H, alkyl, substituted alkyl, aryl, or substituted aryl as set forth herein.

As used herein, the term “carbocycle” refers to a saturated, unsaturated, or aromatic group having a single ring or multiple condensed rings composed of linked carbon atoms. The ring(s) can optionally be unsubstituted or substituted with, e.g., halogen, lower alkyl, alkoxy, alkylthio, acetylene, amino, amido, carboxyl, hydroxyl, aryl, aryloxy, heterocycle, hetaryl, substituted hetaryl, nitro, cyano, thiol, sulfamido, and the like.

As used herein, the term “cycloalkenyl” refers to cyclic ring-containing groups containing in the range of 3-20 carbon atoms and having at least one carbon-carbon double bond, and “substituted cycloalkenyl” refers to cycloalkenyl groups further bearing one or more substituents as set forth herein.

As used herein, the term “cycloalkyl” refers to a monocyclic or polycyclic alkyl group containing 3-15 carbon atoms, and “substituted cycloalkyl” refers to cycloalkyl groups further bearing one or more substituents as set forth herein.

As used herein, the term “cycloalkylene” refers to divalent ring-containing groups containing in the range of about 3-12 carbon atoms, and “substituted cycloalkylene” refers to cycloalkylene groups further bearing one or more substituents as set forth herein.

As used herein, the term “guanidinyl” denotes the group —N═C(NH₂)₂ and “substituted guanidinyl” denotes the group —N═C(NR₂)₂, where each R is independently H, alkyl, substituted alkyl, aryl, or substituted aryl as set forth herein.

As used herein, the term “halo” or “halogen” refers to all halogens, i.e., chloro (Cl), fluoro (F), bromo (Br), and iodo (I).

As used herein, the term “heteroaryl” refers to a monocyclic aromatic ring structure containing 5 or 6 ring atoms, or a bicyclic aromatic group having 8-10 atoms, containing one or more, preferably 1-4, more preferably 1-3, even more preferably 1-2 heteroatoms independently selected from the group O, S, and N, and optionally substituted with 1-3 groups or substituents of halo, hydroxy, alkoxy, alkylthio, alkylsulfinyl, alkylsulfonyl, acyloxy, aryloxy, heteroaryloxy, amino optionally mono- or di-substituted with alkyl, aryl or heteroaryl groups, amidino, urea optionally substituted with alkyl, aryl, heteroaryl, or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, or the like. Heteroaryl is also intended to include oxidized S or N, such as sulfinyl, sulfonyl, and N-oxide of tertiary ring nitrogen. A carbon or nitrogen atom is the point of attachment of the heteroaryl ring structure such that a stable aromatic ring is retained. Examples of heteroaryl groups are phthalimide, pyridinyl, pyridazinyl, pyrazinyl, quinazolinyl, purinyl, indolyl, quinolinyl, pyrimidinyl, pyrrolyl, oxazolyl, thiazolyl, thienyl, isoxazolyl, oxathiadiazolyl, isothiazolyl, tetrazolyl, imidazolyl, triazinyl, furanyl, benzofuryl, indolyl, and the like. A substituted heteroaryl contains a substituent attached at an available carbon or nitrogen to produce a stable compound.

As used herein, the term “substituted heteroaryl” refers to a heterocycle optionally mono or poly substituted with one or more functional groups, e.g., halogen, lower alkyl, lower alkoxy, alkylthio, acetylene, amino, amido, carboxyl, hydroxyl, aryl, aryloxy, heterocycle, substituted heterocycle, hetaryl, substituted hetaryl, nitro, cyano, thiol, sulfamido, and the like.

As used herein, the term “heteroarylcarbonylamino” denotes the group —NR^{q}C(O)R^{r}, where R^{q} is hydrogen or lower alkyl, and R^{r} is optionally substituted aryl.

As used herein, the term “heteroaryloxy” denotes the group —OHet, where Het is an optionally substituted heteroaryl group.

As used herein, the term “heteroarylsulfonylamino” denotes the group —NR^{q}S(O)₂R^{s}, where R^{q} is hydrogen or lower alkyl and R^{s} is optionally substituted heteroaryl.

As used herein, the term “heterocycle” refers to a saturated, unsaturated, or aromatic group having a single ring (e.g., morpholino, pyridyl or furyl) or multiple condensed rings (e.g., naphthpyridyl, quinoxalyl, quinolinyl, indolizinyl or benzo[b]thienyl) and having carbon atoms and at least one hetero atom, such as N, O or S, within the ring, which can optionally be unsubstituted or substituted with, e.g., halogen, lower alkyl, lower alkoxy, alkylthio, acetylene, amino, amido, carboxyl, hydroxyl, aryl, aryloxy, heterocycle, hetaryl, substituted hetaryl, nitro, cyano, thiol, sulfamido, and the like.

As used herein, the term “substituted heterocycle” refers to a heterocycle substituted with 1 or more, e.g., 1, 2, or 3, substituents selected from the group consisting of optionally substituted alkyl, optionally substituted alkenyl, optionally substituted alkynyl, halo, hydroxy, alkoxy, alkylthio, alkylsulfinyl, alkylsulfonyl, acyloxy, aryl, substituted aryl, aryloxy, heteroaryloxy, amino, amido, amidino, urea optionally substituted with alkyl, aryl, heteroaryl or heterocyclyl groups, aminosulfonyl optionally N-mono- or N,N-di-substituted with alkyl, aryl or heteroaryl groups, alkylsulfonylamino, arylsulfonylamino, heteroarylsulfonylamino, alkylcarbonylamino, arylcarbonylamino, heteroarylcarbonylamino, acyl, carboxyl, heterocycle, substituted heterocycle, hetaryl, substituted hetaryl, nitro, cyano, thiol, sulfonamido, and oxo, attached at any available point to produce a stable compound.

As used herein, the term “hydrocarbylcarbonyl” refers to —C(O)-hydrocarbyl groups containing 2-20 carbon atoms and “substituted hydrocarbylcarbonyl” refers to hydrocarbylcarbonyl groups further bearing one or more substituents as set forth herein.

As used herein, the term “hydrocarbyloxycarbonyl” refers to —C(O)—O-hydrocarbyl containing 2-20 carbon atoms and “substituted hydrocarbyloxycarbonyl” refers to hydrocarbyloxycarbonyl groups further bearing one or more substituents as set forth herein.

As used herein, the term “hydrocarbylcarbonyloxy” refers to —O—C(O)-hydrocarbyl groups 2-20 carbon atoms and “substituted hydrocarbylcarbonyloxy” refers to hydrocarbylcarbonyloxy groups further bearing one or more substituents as set forth herein.

As used herein, the term “hydroxyl” or “hydroxy” refers to the group —OH.

As used herein, the term “organosulfinyl” denotes the group —S(O)-organo, where organo embraces alkyl-, alkoxy-, alkylamino-, and aryl moieties, as well as substituted alkyl-, alkoxy-, alkylamino-, and aryl moieties.

As used herein, the term “organosulfonyl” denotes the group —S(O)₂-organo, where organo embraces alkyl-, alkoxy- and alkylamino-moieties, as well as substituted alkyl-, alkoxy- or alkylamino-moieties.

As used herein, the term “oxo” refers to an oxygen substituent double bonded to the attached carbon.

As used herein, the term “sulfinyl” denotes the group —S(O)—.

As used herein, the term “substituted sulfinyl” denotes the group —S(O)R^{t}, where R^{t} is lower alkyl, substituted lower alkyl, cycloalkyl, substituted cycloalkyl, cycloalkylalkyl, substituted cycloalkylalkyl, heterocyclyl, substituted heterocyclyl, heterocyclylalkyl, substituted hetereocyclylalkyl, aryl, substituted aryl, heteroaryl, substituted heteroaryl, heteroaralkyl, substituted heteroaralkyl, aralkyl, or substituted aralkyl.

As used herein, the term “sulfonyl” denotes the group —S(O)₂—.

As used herein, the term “substituted sulfonyl” denotes the group —S(O)₂R^{t}, where R^{t} is lower alkyl, substituted lower alkyl, cycloalkyl, substituted cycloalkyl, cycloalkylalkyl, substituted cycloalkylalkyl, heterocyclyl, substituted heterocyclyl, heterocyclylalkyl, substituted hetereocyclylalkyl, aryl, substituted aryl, heteroaryl, substituted heteroaryl, heteroaralkyl, substituted heteroaralkyl, aralkyl, or substituted aralkyl.

As used herein, the term “sulfonylamino” denotes the group —NR^{q}S(O)₂— where R^{q} is hydrogen or lower alkyl.

As used herein, the term “substituted sulfonylamino” denotes the group —NR^{q}S(O)₂R^{u}, where R^{q} is hydrogen or lower alkyl and R^{u} is lower alkyl, substituted lower alkyl, cycloalkyl, substituted cycloalkyl, heterocyclyl, substituted heterocyclyl, aryl, substituted aryl, heteroaryl, substituted heteroaryl, heteroaralkyl, substituted heteroaralkyl, aralkyl, or substituted aralkyl.

As used herein, the term “sulfuryl” denotes the group —S(O)₂—.

As used herein in connection with numerical values, the term “approximately” or “about” means 10% of the indicated value. For example, “about 3%” would encompass 2.7-3.3% and “about 10%” would encompass 9-11%. Moreover, where “about” is used herein in conjunction with a quantitative term it is understood that in addition to the value plus or minus 10%, the exact value of the quantitative term is also contemplated and described. For example, the term “about 3%” expressly contemplates, describes and includes exactly 3%.

### BRIEF DESCRIPTION OF THE FIGURES

FIG. 1 is a schematic representation of the mechanism of Ligase Chain Reaction (LCR).

FIG. 2 is a schematic representation of a possible mechanism illustrating how nucleic acid ligation can be impaired by substituted ligase cofactors ATP or NAD+ prior to Hot Start activation. In FIG. 2, Z represents a thermolabile substitution group; A^{Z}TP or N^{Z}AD+ represent SCs; ATP or NAD+ represent unsubstituted cofactors; solid lines represent unsubstituted acceptor and donor probes; the circle represents DNA ligase; and the striped line represents DNA template.

FIG. 3 is a schematic representation of a possible mechanism illustrating how nucleic acid ligation can be impaired by SAPs prior to HS LCR activation. In FIG. 3, a vertical line represents a thermolabile substitution group; a vertical bar intersecting a horizontal line at the left hand side represents a SAP; solid lines represent unsubstituted acceptor and donor probes; a circle represents DNA ligase; and a striped line represents DNA template.

FIG. 4 is a schematic representation of the mechanism of phosphodiester bond formation by ATP-dependent and NAD+-dependent DNA ligase reactions.

FIG. 5 is a schematic representation of the scheme of synthesis of substituted ATP.

FIG. 6 is a schematic representation of the scheme of synthesis of sugar substituted NAD+ derivatives using Route 1 described herein.

FIG. 7 shows phosphoramidite monomers, of compounds 1(a-d) having nucleoside bases (a) N⁶-phenoxyacetyladenine; (b) N²-phenoxyacetylguanine; (c) N⁴-acetylcytosine; and (d) thymine, for incorporation of an OXT substitution group in acceptor and donor probes.

FIG. 8 shows kinetic curves of cleavage of 2′- and 3′-THF substitution groups from 2′,3′-bis-THF ATP. In FIG. 8, (∘) represents the kinetic curve of bis-THF-modified 2′,3′-THF ATP; (□) represents the kinetic curve of mono-THFmodified 2′- and 3′-THF ATPs; (e) represents the kinetic curve of unmodified ATP.

FIG. 9 shows kinetic curves of cleavage of 2′- and 3′-TBE modification groups from 2′,3′-bis-TBE ATP. In FIG. 9, (∘) represents the kinetic curve of bis-modified 2′,3′-THF ATP; (□) represents the kinetic curve of monomodified 2′- and 3′-THF ATPs; (•) represents the kinetic curve of unmodified ATP.

FIG. 10 shows kinetic curves of cleavage of the OXT group from OXT-SAPs in ligase buffer. In FIG. 10, (∘) represents the kinetic curve of OXT-substituted probe; (•) represents the kinetic curve of phosphodiester unsubstituted probe.

FIG. 11 shows SYBR gold stained PAGE analysis of the ligase reaction with acceptor and donor probes, 2′,3′-bis-TBE-substituted ATP cofactor and T4 DNA ligase for 10 and 60 min at room temperature. All oligonucleotides are in equimolar ratio. Gel: TBE-Urea 15% polyacrylamide gel, run at 60-70° C. Lanes: 1 is 50 bp ladder; lanes 3-6 are preheated TBE-ATP; lanes 7-8 are original TBE-ATP; lanes 9-10 are no cofactor added; and lanes 11-12 are unsubstituted ATP.

FIG. 12 shows SYBR gold stained PAGE analysis of the ligase reaction with Tth DNA ligase, donor probe, A-T-matched (lanes: 1-4,7,8,11,12) or G-T-mismatched (lanes: 5,6,9,10,13,14) template and OXT-substituted (*) or unsubstituted acceptor probe. All oligonucleotides are in equimolar ratio. Gel: TBE-Urea 15% polyacrylamide gel, run at 60-70° C. (Bands of short OXT-substituted and unsubstituted acceptor probes are significantly diffused due to high gel temperature and therefore are not stained efficiently). Ligation conditions: 1 μM synthetic template, 1 μM of each of donor and acceptor probes, 50 U Tth DNA ligase, volume: 12 μL. Lanes 1, 3, 5, 7, 9, 11 and 13: unmodified phosphodiester acceptor probe; lanes 2, 4, 6, 8, 10, 12, and 14: OXT-modified acceptor probe.

FIG. 13 shows blunt-ended and shift-ended probe geometries for LCR and possible “false-positive” ligation product for blunt-ended probe geometry.

FIG. 14 shows PAGE analysis of ligation product for matched (M) and mismatched (MM) DNA templates. Ligation conditions: 40 units Taq DNA Ligase and 1× NEB buffer in 12 μL at 60° C. for 1 hour.

FIG. 15 shows a typical temperature cycling sequence for Hot Start Real-Time LCR.

FIG. 16 shows a Real-Time LCR with PDE or OXT WT probes with WT (match) and G551D (mismatch) templates. Ligation conditions: NEB 1.25×Taq DNA Ligase buffer, 300 000 copies of synthetic dsDNA template (19+21 or 20+22), 0.1 μM of each of donor and acceptor probes, 20 U Taq DNA ligase, volume: 20 mL; SYBR Green detection. Thermal cycling conditions: 95° C. (5 min); [95° C. (30 sec), 55° C. (5 sec), 74° C. (30 sec)] 50×.NTC—no template control

FIG. 17 shows a Real-Time LCR with shifted PDE or OXT acceptor probes on WT (match) and G551D (mismatch) templates. Ligation conditions: NEB 1× Taq DNA Ligase buffer, 3 millions copies of synthetic dsDNA template (19+21 or 20+22), 0.1 μM of each of donor and acceptor probes, 20 U Taq DNA ligase, volume: 20 μL; SYBR Green detection. Thermal cycling conditions: 95° C. (5 min); [95° C. (30 sec), 55° C. (5 sec), 74° C. (30 sec)] 50×. NTC—no template control.

FIG. 18 shows Hot Start Real-Time ligation reaction with TBE-substituted NAD⁺ and unmodified probes. Ligation conditions: 300,000 copies of synthetic dsDNA template (19+21), 0.1 uM of each unmodified probe (1+8+12+16; PDE set), 20 units of Taq DNA ligase, 1 mM TBE-substituted NAD⁺, volume: 20 uL; SYBR Green detection; NTC: no template control. Thermal cycling conditions: 95° C. (5 min); [95° C. (30 sec); 55° C. (5 sec); 74° C. (30 sec)]×40 cycles. NTC—no template control.

FIG. 19 shows the synthesis of 2′,3′-bis-substituted-N⁶-phenoxyacetyl-adenosine 5′-triphosphates and NAD⁺ analogues.

FIG. 20 shows the structure of a substituted ATP cofactor analog containing a thermolabile group on the sugar and gamma-phosphate moieties wherein R and R′ are a tetrahydrofuranyl (THF), [2-(tert-butoxy)]ethyl (TBE) and/or [2-cyclohexoxy)]ethyl (CHE) groups).

### DETAILED DESCRIPTION OF THE INVENTION

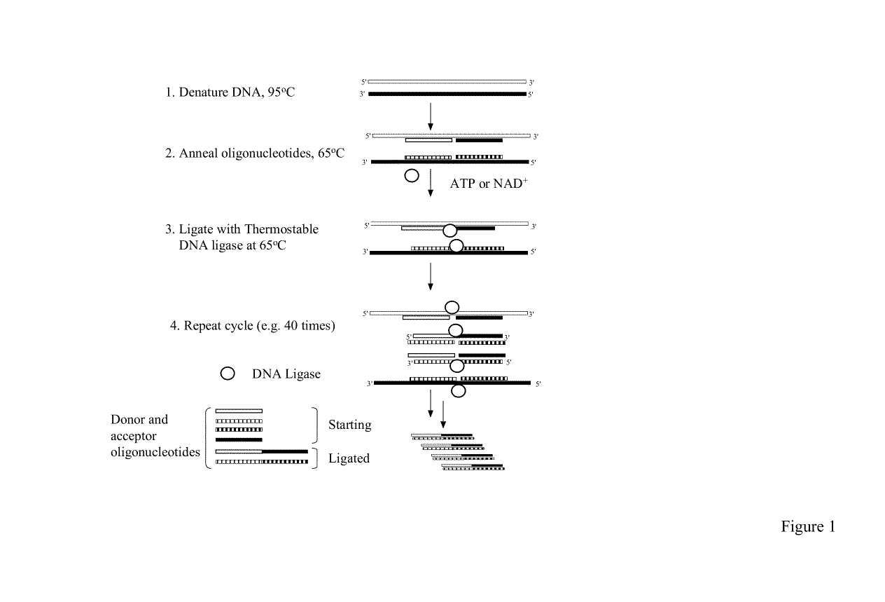

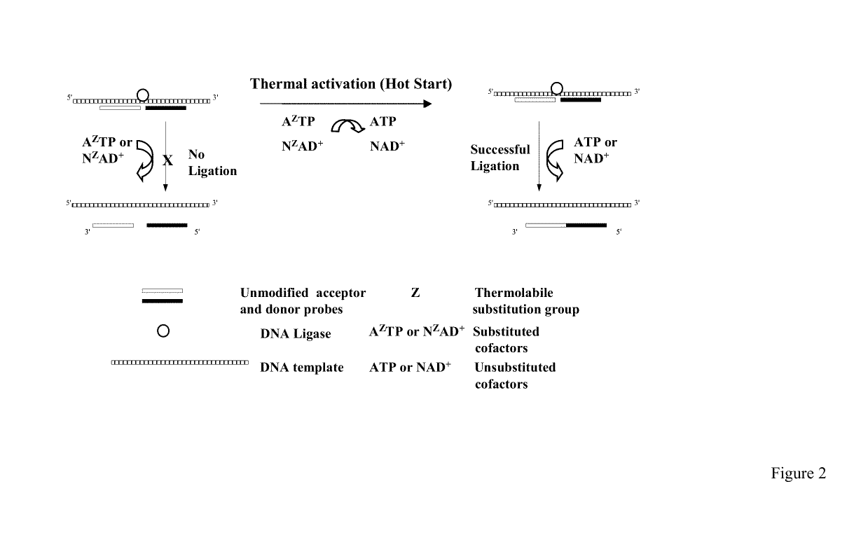

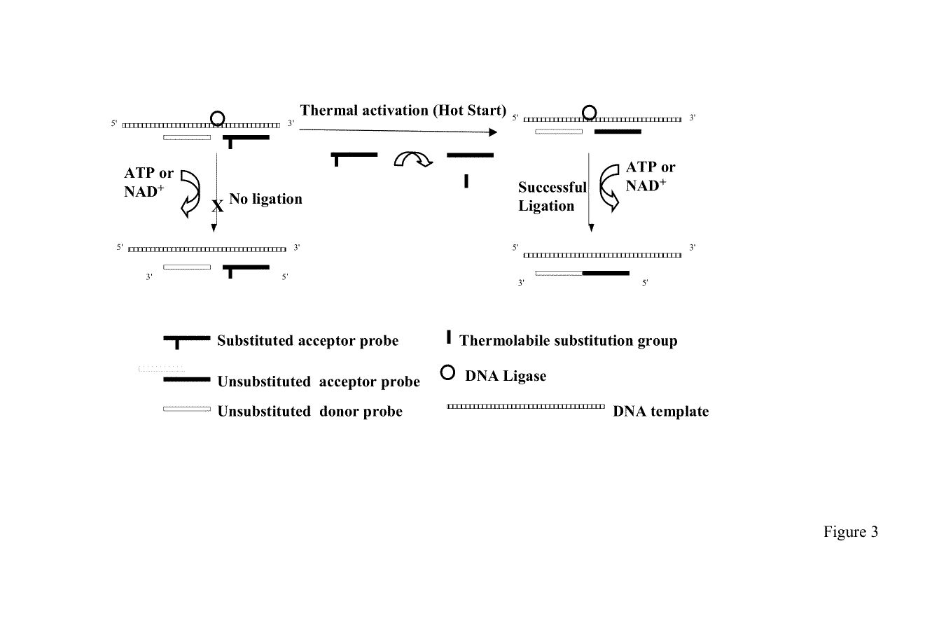

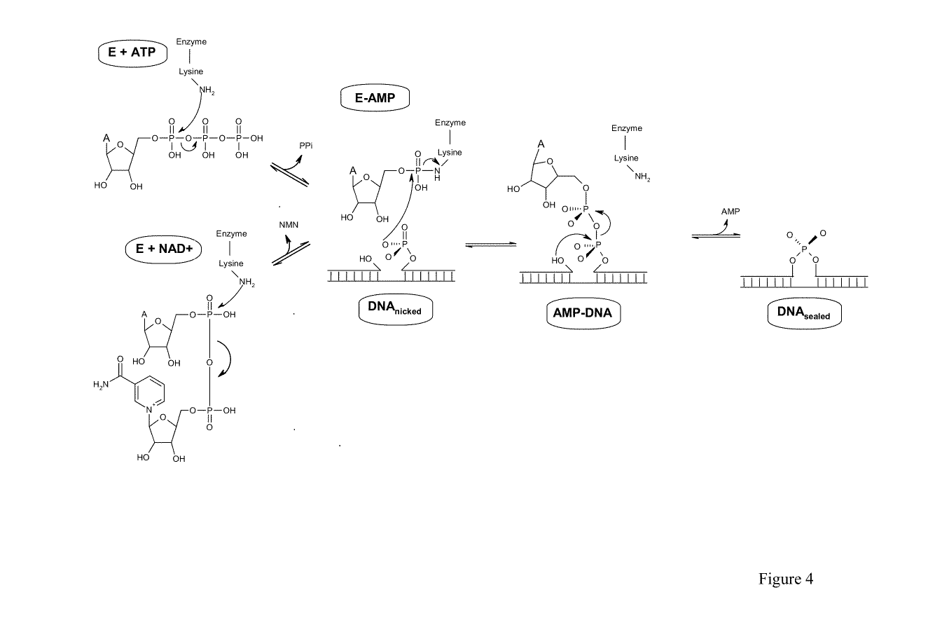

Ligase Chain Reaction (LCR) can be used to both amplify nucleic acid sequence and discriminate base pair mismatches. LCR generally includes the use of four oligonucleotides, e.g., two pairs of a donor probe and an acceptor probe. One donor probe and acceptor probe pair are adjacent oligonucleotides that hybridize to a first strand of a target DNA sequence. The second donor probe and acceptor probe pair are adjacent oligonucleotides that hybridize to the complementary strand of the target DNA sequence. LCR is based on a template-dependent sequence specific ligation of a pair of adjacent donor and acceptor probes on DNA template to form a ligation product, provided that there is complementarity at the junction of an oligonucleotide pair to the DNA strand. Once ligation occurs, the complex of DNA template and ligated product dissociates during the following heat denaturing step and each newly formed strand of ligated product can serve as a template for the next cycle of ligation allowing for exponential nucleic acid amplification (see FIG. 1).

DNA ligases, mesophili and thermophilic, have been found to tolerate a variety of nucleic acid substrate mismatches (see e.g., Wu et al., Gene 76, 245-254 (1989); Landegren, et al., Science 241, 1077-1080 (1988); Alexander, et al., Nucl. Acids Res. 31, 3208-3216 (2003)) and T4 DNA ligase (Showalter, et al., Chem. Rev. 106, 340-360 (2006)).

Small quantities of single stranded DNA (ssDNA) may be present in analytical samples due to reasons such as poor quality of starting DNA samples (see e.g., Wang, et al., J. Mol. Diagn. 9, 441-451 (2007)), sub-optimal performance of DNA isolation/purification procedures (see e.g., Ward, et al., Biochemistry, 24, 5803-5809 (1985); Tan et al., J. of Biomed. Biotechnology (2009) Article ID 574398, pp. 1-10) or a single stranded cDNA template generated by reverse transcription. These ssDNA segments can serve as mismatched templates for off-target ligation at low stringency conditions before LCR temperature cycling starts and can result in a “false-positive” signal.

DNA ligase has also been implicated in a number of atypical joining reactions, including intramolecular loop formation (Western et al., Nucl. Acids Res. 19, 809-813 (1991)), template-independent reactions (Barringer, et al., Gene 89, 117-122 (1990); Kuhn et al., FEBS J. 272, 5991-6000 (2005)), and joining of non-overlapping blunt-ended duplexes (Cao, Trends Biotechnol. 22, 38-44 (2004); Barringer, et al., Gene 89, 117-122 (1990)). Template independent ligation of blunt-ended duplexes of complementary acceptor and donor probes can serve as template for further ligation steps and result in accumulation of a “false-positive” signal (Abravaya, et al., Nucl. Acids Res. 23, 675-682 (1995)). Wild-type DNA ligases such as Thermus thermophilus (Tth) and Thermus aquaticus (Taq) may not have the specificity required for certain diagnostic detection assays (Barany, Proc. Natl. Acad. Sci. USA 88, 189-193 (1991)).

Ligase mediated nucleic acid amplification reaction, such as LCR, uses thermal cycling protocol similar to PCR. LCR involves several key steps (FIG. 1): (a) adenylation of the ligase enzyme using ATP or NAD+ cofactor; (b) heat denaturation step that separates two complementary strands of dsDNA; (c) annealing step for hybridization of a pair of donor and acceptor probes to a target nucleic acid followed by (d) transfer of the adenylate residue from adenylated lisase to the donor strand in ligation complex forming adenylated donor intermediate and (e) ligase mediated formation of phosphodiester linkage between adenylated donor and acceptor probe strands forming a ligated product which is a complementary copy of the target nucleic aid sequence of interest. Steps (a)-(e) of the first cycle are repeated in the second cycle and the ligated product formed in the first cycle of ligation during step (e) serves as novel template for a pair of donor and acceptor probes (FIG. 1). This repeating process provides exponential amplification of signal, analogous of PCR amplification.

Various approaches have been described for improving ligation fidelity. For example, Lebedev, et al., PCT/US2010/41069 disclose permanent chemical modification of donor oligonucleotides, acceptor oligonucleotides and ligase cofactors such as NAD+ and ATP. Luo, J., et al., 24 Nucleic Acids Res, 3079-3085 (1996) disclose modifying the third nucleotide upstream from the 3′-OH, acceptor with universal base 3-nitropyrrole and site directed mutagenesis of the ligase protein. Tong, J., et al., 27 Nucleic Acids Res, 788-794 (1999); Feng, H., et al., 43 Biochemistry, 12648-12659 (2004); Jeon, H., et al., 237 FEMS Microbiol Lett., 111-118 (2004); Lim, J., et al., 388 Arch Biochem Biophys., 253-260 (2001); and Luo, J., et al., 24 Nucleic Acids Res, 3071-3078 (1996) disclose mutating amino acid residues in the DNA ligase. Cao, W., 22 Trends Biotechnol., 38-44 (2004) disclose using an endonuclease in the ligation reaction. Egholm, M., et al., U.S. Pat. No. 6,297,016 disclose acceptor modifications. Fung, S., et al., U.S. Pat. No. 5,593,826 discloses 3′-NH₂ modified acceptor probes. Bandaru, R., et al., U.S. Pat. Nos. 6,811,986 and 6,635,425 discloses use of 5′-thiophosphates in the donor (5′-phosphate) strand. Jeng et al., J. Supramol. Struct., 448-468 (1975) disclose synthesis of 3′-arylazido ATP analogs and their use as photoaffinity labels for myosin ATPase. Similar compounds were prepared and tested in other ATPase systems (Schafer, et. al., 87 FEBS Lett., 318-322 (1978); Lunardi, et. al., 20 Biochemistry, 473-480 (1981)).

Fang, et al., Hum. Mutation. 6, 144-151 (1995); Barany, et al., WO 97/31256); Barany et al, U.S. Pat. No. 6,576,453) disclose DNA ligase added to a ligation mixture after the initial heat-denaturing step. In another approach (Skaug and Berg, U.S. Pat. No. 6,114,155; Balles et al., J. Mol. Gen. Genet. 245, 734-740 (1994)), a wax layer was used as a physical barrier to temporarily separate DNA ligase from all other components of the ligation mixture until an initial heat denaturing step melts the wax barrier.

Another variant of ligase mediated nucleic acid amplification reaction, such as LCR, uses a pre-formed adenylate-donor intermediate and does not require ligase cofactor. In this case, principal scheme of LCR experiment involves fewer key steps (a) heat denaturation step that separates two complementary strands of dsDNA; (b) annealing step for hybridization of the first pair of acceptor probe and adenylated donor intermediate to a target nucleic acid followed by (c) ligase mediated formation of phosphodiester linkage between adenylated donor and acceptor probe strands forming a ligated product which is a complementary copy of the target nucleic aid sequence. Steps (a)-(c) of the first cycle are repeated in the second cycle with exception that ligated product formed in the first cycle of ligation during step (c) serves as novel template for a pair of adenylated donor intermediate and acceptor probes. This repeating process provides exponential amplification of signal, analogous of PCR amplification.

During preparation of a LCR mixture, before LCR starts, the temperature conditions for hybridization of donor and acceptor strands to nucleic acid template are not stringent. Therefore, at these low stringency conditions a ligation of donor and acceptor probes or adenylate-donor intermediate and acceptor probe can occur even when there is a mismatch (or partial non-complementary) between the oligonucleotide probes and nucleic acid template or, sometimes, even in the absence of template nucleic acid.

Exemplary ligation methods suitable for use with the heat activatable SLCs provided herein include oligonucleotide ligation assay (OLA) (Landegren, U., et al. 241 Science, 1077-1080 (1988)), ligase chain reaction (LCR) (Wiedmann, M., et al. 3 Genome Biol, S51-64 (1994)), Ligase Mediated PCR (LM-PCR) (Mueller, P. R., et al. 246 Science, 780-786 (1989), Pfeifer, G. P., et al. 246 Science, 810-813 (1989)), PCR ligation detection reaction (PCR-LDR) (Cheng, Y. W., et al. 16 Genome Res, 282-289 (2006)), Padlock probes (Antson, D., et al. 28 Nucleic Acids Res, e58 (2000)), PCR oligonucleotide ligation assay (PCR-OLA) (Delahunty, C., et al. 58 Am J Hum Genet, 1239-1246 (1996)), gap LCR approach (Abravaya, K., et al. 23 Nucleic Acids Res, 675-682 (1995)), SNPlex (De la Vega, F. M., et al. 73 Mutat Res, 111-135 (2005), Livak, K. J. 14 Genet Anal, 143-149 (1999)), MLPA (multiplex ligation-dependent probe amplification) (Schouten, J. P., et al. 30 Nucleic Acids Res, e57 (2002)), GoldenGate Genotyping Assay (Fan, J. B., et al. 68 Cold Spring Harb Symp Quant Biol, 69-78 (2003), Oliphant, A., et al. Suppl Biotechniques, 56-58, 60-51 (2002), Shen, R., et al. 573 Mutat Res, 70-82 (2005)), and Molecular Inversion Probe Assay (Fodor, S. P., et al. 251 Science, 767-773 (1991), Matsuzaki, H. S., et al. 1 Nat Methods, 109-111 (2004), Matsuzaki, H., et al. 14 Genome Res, 414-425 (2004), Pease, A. C., et al. 91 Proc Natl Acad Sci USA, 5022-5026 (1994)), proximity ligation (Gustafsdottir, S., et al. 345 Anal Biochem, 2-9 (2005), Soderberg, O., et al. 28 Genet Eng (N Y), 85-93 (2007)), and next-generation sequencing by ligation.

Exemplary ligation-based approaches for sequence detection suitable for use with the heat activatable SLCs provided herein include those as described in Barany, F., et al. U.S. Pat. Nos. 7,244,831; 6,312,892 and the use of high fidelity thermostable ligases (U.S. Pat. No. 6,949,370), LDR and PCR coupling (Barany, F., et al. U.S. Pat. Nos. 7,097,980; 6,797,470; 6,268,148; 6,027,889; 7,166,434), ligation using an endonuclease (Barany, F., et al. U.S. Pat. Nos. 7,198,894; 7,014,994), OLA/PCR (Eggerding, F., U.S. Pat. Nos. 5,912,148; 6,130,073), ligation/amplification (Lao, K. Q. U.S. Pat. No. 7,255,994), stepwise ligation and cleavage (Brenner, S., et al. U.S. Pat. Nos. 5,714,330; 5,552,278), proximity ligation (Gustafsdottir, S., et al. 345 Anal Biochem, 2-9 (2005), Soderberg, O., et al. 28 Genet Eng (N Y), 85-93 (2007), Fredriksson, S., et al. 20 Nat Biotechnol, 473-477 (2002)), proximity ligation for pathogen detection (Gustafsdottir, S. M., et al. 52 Clin Chem, 1152-1160 (2006)), cytokines detection (Gullberg, M., et al. 101 Proc Natl Acad Sci USA, 8420-8424 (2004)), spore detection (Pai, S., et al. 33 Nucleic Acids Res, e162 (2005)), and cancer biomarker detection (Fredriksson, S., et al. 4 Nat Methods, 327-329 (2007)), and proximity ligation for measuring strength of protein-DNA interactions (Gustafsdottir, S., et al. 345 Anal Biochem, 2-9 (2005), Schallmeiner, E., et al. 4 Nat Methods, 135-137 (2007)).

Exemplary ligation-based diagnostic assays suitable for use with the heat activatable SLCs provided herein include detection of HIV drug resistant strains (Lalonde, M., et al. 45 J Clin Microbiol, 2604-2615 (2007)) multiplexed detection of allele-specific products (Macdonald, S. J., et al. 6 Genome Biol, R105 (2005)), SNP detection by ligation including oligonucleotide ligation assay (OLA) (Landegren, U., et al. 241 Science, 1077-1080 (1988)), ligase chain reaction (LCR) (Wiedmann, M., et al. 3 Genome Biol, S51-64 (1994)), SNP detection using combinations of ligation and PCR including Ligase Mediated PCR (LM-PCR) (Mueller, P. R., et al. 246 Science, 780-786 (1989), Pfeifer, G. P., et al. 246 Science, 810-813 (1989)), PCR ligation detection reaction (PCR-LDR) (Cheng, Y. W., et al. 16 Genome Res, 282-289 (2006)), Padlock probes (Antson, D., et al. 28 Nucleic Acids Res, e58 (2000)), PCR oligonucleotide ligation assay (PCR-OLA) (Delahunty, C., et al. 58 Am J Hum Genet, 1239-1246 (1996)), and a gap LCR approach (Abravaya, K., et al. 23 Nucleic Acids Res, 675-682 (1995)), SNPlex (De la Vega, F. M., et al. 573 Mutat Res, 111-135 (2005), Livak, K. J. 14 Genet Anal, 143-149 (1999)), MLPA (multiplex ligation-dependent probe amplification) (Schouten, J. P., et al. 30 Nucleic Acids Res, e57 (2002)), Illumina's GoldenGate Genotyping Assay (Fan, J. B., et al. 68 Cold Spring Harb Symp Quant Biol, 69-78 (2003), Oliphant, A., et al. Suppl Biotechniques, 56-58, 60-51 (2002), Shen, R., et al. 573 Mutat Res, 70-82 (2005)), and Molecular Inversion Probe Assay on Affymetrix GeneChip arrays (Fodor, S. P., et al. 251 Science, 767-773 (1991), Matsuzaki, H., et al. 1 Nat Methods, 109-111 (2004), Matsuzaki, H., et al. 14 Genome Res, 414-425 (2004), Pease, A. C., et al. 91 Proc Natl Acad Sci USA, 5022-5026 (1994)).

Additional exemplary ligation assays suitable for use with the heat activatable SLCs provided herein include traditional Sanger dideoxy sequencing (Sanger, F., et al. 74 Proc Natl Acad Sci USA, 5463-5467 (1977) and next generation sequencing assay such as 454 Sequencing System, the Illumina Genome Analyzer, Knome's KnomeCOMPLETE™ genome sequencing service, and the ABI SOLiD™ System sequencing technology and other sequencing by ligation assays (Ronaghi, M. 11 Genome Res, 3-11 (2001), Mirzabekov, A. 12 Trends Biotechnol, 27-32 (1994), Schmalzing, D., et al. 20 Electrophoresis, 3066-3077).

The methods and compositions provided herein will now be described in greater detail by reference to the following non-limiting examples.

### Example 1

### Synthesis of: 2′,3′-O-bis-[2-(tert-butoxy)]ethyl-adenosine-5′-triphosphate, 2′,3′-O-bis-tetrahydrofuranyl-adenosine-5′triphosphates and 2′,3′-O-bis-[2-(cyclohexyl)]ethyl-adenosine-5′triphosphates (TBE-, THF- and CHE-substituted ATP)

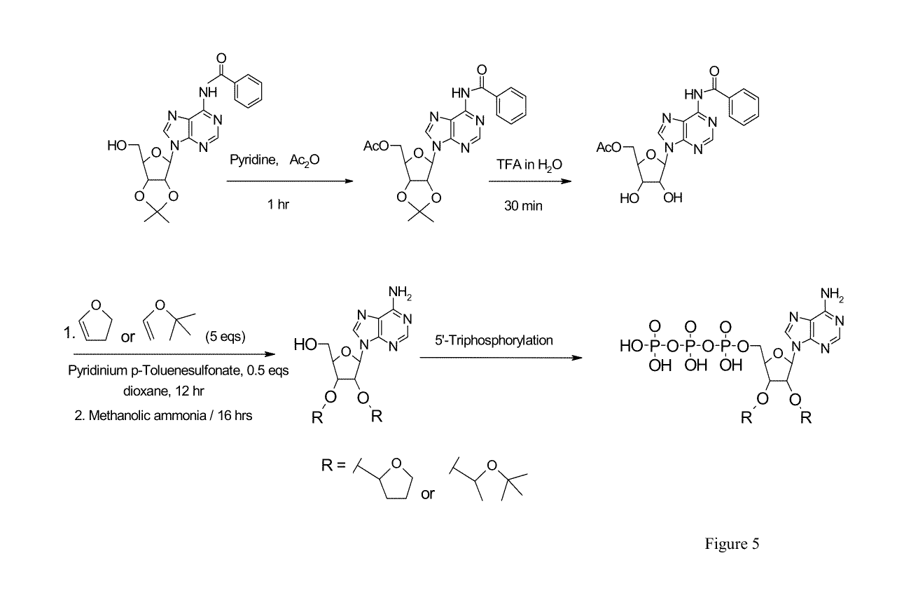

### Synthesis of 5′-O-acetyl-N⁶-benzoyl-adenosine (FIG. 5)

2′,3′-O-isopropylidene-N⁶-benzoyl-adenosine, 0.6 g (2.2 mmol), was co-evaporated with dry pyridine, dissolved in 5 mL of dry pyridine and treated with 2 mL of acetic anhydride for 1 hour. The excess of acetic anhydride was quenched by 2 mL of methanol, and after 5 min a saturated aqueous solution of sodium bicarbonate (20 mL) was added. The mixture was extracted twice with 30 mL of dichloromethane, organic layers were combined together, dried over Na₂SO₄, filtered, and evaporated to dryness. The residue was dissolved in dichloromethane and purified on silica gel column (20 g) using 3% methanol in dichloromethane as eluting solution.

Fractions containing 5′-O-acetyl-2′,3′-O-isopropylidene-N⁶-benzoyl-adenosine were pooled, dried down on rotary evaporator and treated with 10 mL of 80% trifluoroacetic acid in water for 0.5 hour. The reaction mixture was then dried down and co-evaporated on rotary evaporator with dichloromethane (30 mL) and toluene (20 mL). The residue was dissolved in dichloromethane (20 mL) and extracted twice with aqueous sodium bicarbonate (20 mL). Combined organic layers were dried over anhydrous sodium sulfate, filtered, concentrated on rotary evaporator and purified on silica gel column (15 g) using 5% methanol in dichloromethane as eluting solution. Fractions containing 5′-O-acetyl-N⁶-benzoyl-adenosine were pooled and dried down to obtain 0.140 g of product (35%).

### Synthesis of 2′,3′-O-bis-substituted adenosine (FIG. 5

Isolated 5′-O-acetyl-N-benzoyladenosine was reacted with 5 eqs of 2,3-dihydrofuran or tert-butyl vinyl ether or cyclohexyl vinyl ether in the presence of 0.5 eqs of pyridinium p-toluenesulfonate in dioxane for 16 hours at room temperature. Subsequent treatment with methanolic ammonia to remove 5′-O-acetyl and N⁶-benzoyl protecting group produced 2′,3′-O-bis-tetrahydrofuranyl (THF) or 2′,3′-O-bis-[2-(tert-butoxy)]ethyl (TBE) or 2′,3′-O-bis-[2-(cyclohexyl)]ethyl (CHE) derivatives of adenosine, respectively. The compounds were isolated on silica gel column using 0.1% TEA/2% MeOH/97.9% CH₂Cl₂ as eluting solution.

### Synthesis of 2′,3′-O-bis-substituted-adenosine-5′-triphosphate (FIG. 5

2′,3′-bis substituted adenosine 5′-triphosphates were prepared from corresponding 2′,3′-substituted adenosines according to the Ludwig-Eckstein procedure (Ludwig, J. Org. Chem., 54, 631-635 (1989)) as follows.

2′,3′-O-bis-substituted adenosine was reacted with 1.1 equiv. of 2-chloro-4H-1,3,2-benzodioxaphosphorin-4-one in dioxane-pyridine solution followed by reaction with 1.6 equiv. of tributylammonium pyrophosphate, subsequent oxidation of P(III) to P(V) with iodine solution in pyridine-water (98:2), and final treatment with aqueous 1M triethylammonium bicarbonate. The resulting 2′,3′-O-bis-substituted ATP derivatives were isolated and purified by a combination of anion-exchange and reverse-phase chromatography to obtain 98-99% pure 2′,3′-substituted ATP derivative as sodium salt. Structures of synthesized compounds were confirmed by proton and phosphorus NMR and mass-spectrometry.

### Example 2

### Synthesis of P¹-5′-{2′,3′-O-bis-[2-(tert-butoxy)]ethyl}-nicotinamide riboside-P²-5′-adenosine pyrohosphate and P¹-5′-{2′,3′-O-bis-[2-(cyclohexyl)]ethyl}-nicotinamide riboside-P²-5′-adenosine pyrohosphate (TBE- and CHE-substituted NAD

### Synthesis of 5′-O-acetyl-nicotinamide riboside

Nicotinamide riboside (2.0 mmol) is co-evaporated with dry pyridine (10 mL), dissolved in 5 mL of dry pyridine and treated with 250 mL (2.64 mmol) of acetic anhydride for 4 hours. The mixture is quenched by 10 mL of methanol and evaporated on rotary evaporator to an oil state. The residue is dissolved in 100 mM TEAB, pH 8.5, and purified on reverse phase column (47×300 mm) using gradient of acetonitrile in 100 mM TEAB, pH 8.5, as eluting solution. Fractions containing 5′-O-acetyl-nicotinamide riboside are pooled, dried down on rotary evaporator and co-evaporated with methanol to give pure material. The expected yield is 50%.

### Synthesis of 2′,3′-O-bis-substituted-nicotinamide riboside

Isolated 5′-O-acetyl-nicotinamid riboside (1 mmol) is reacted with 5 eqs of tert-butyl vinyl ether or cyclohexyl vinyl ether in the presence of 0.5 eqs of pyridinium p-toluenesulfonate in dioxane for 4 hours at room temperature. Subsequent treatment with methanolic ammonia to remove 5′-O-acetyl protecting group produces 2′,3′-O-bis-substituted-nicotinamide riboside (TBE-NAR or CHE-NAR, respectively). The mixture is evaporated on rotary evaporator; the residue is dissolved in 100 mM TEAB, pH 8.5, and purified on reverse phase column (47×300 mm) using gradient of acetonitrile in 100 mM TEAB, pH 8.5, as eluting solution. Fractions containing 2′,3′-O-bis-substituted-nicotinamide riboside are pooled, dried down on rotary evaporator and co-evaporated with methanol to give pure material. The expected yield of TBE-NAR and CHE-NAR is 0.5 mmol, 50%.

### Synthesis of 5′-O-phosphoryl-2,3′-O-bis-substituted-nicotinamide riboside

2′,3′-O-bis-substitutednicotinamide riboside 5′-phosphate is prepared from corresponding 2′,3′-O-bis-substitutednicotinamide riboside adenosine as follows. TBE-NAR and CHE-NAR (0.5 mmol) is reacted with 2.0 equiv. of pyridinium 2-cyanoethyl phosphate and DCC in pyridine for 24 hrs at room temperature. Equal volume of 50% pyridine in water is added and mixture is stirred for 20 hrs at room temperature. DCC-urea is filtered and the resulting 2′,3′-O-bis-substitutednicotinamide riboside 5′-phosphate is isolated and is purified by a combination of anion-exchange and reverse-phase chromatography to obtain 98-99% pure 2′,3′-O-bis-[2-(tert-butoxy)]ethyl-nicotinamide riboside 5′-phosphate (TBE-NRP) or 2′,3′-O-bis-[2-(cyclohexoxy)]ethyl-nicotinamide riboside 5′-phosphate (CHE-NRP as triethylammonium salt. The expected yield is 0.15 mmol, 30%.

### Synthesis of P¹-5′-{2′,3′-O-bis-[2-(tert-butoxy)ethyl]}-nicotinamide riboside-P²-5′-adenosine pyrophosphate and P¹-5′-{2′,3′-O-bis-[2-(cyclohexoxy)ethyl]}-nicotinamide riboside-P²-5′-adenosine pyrophosphate

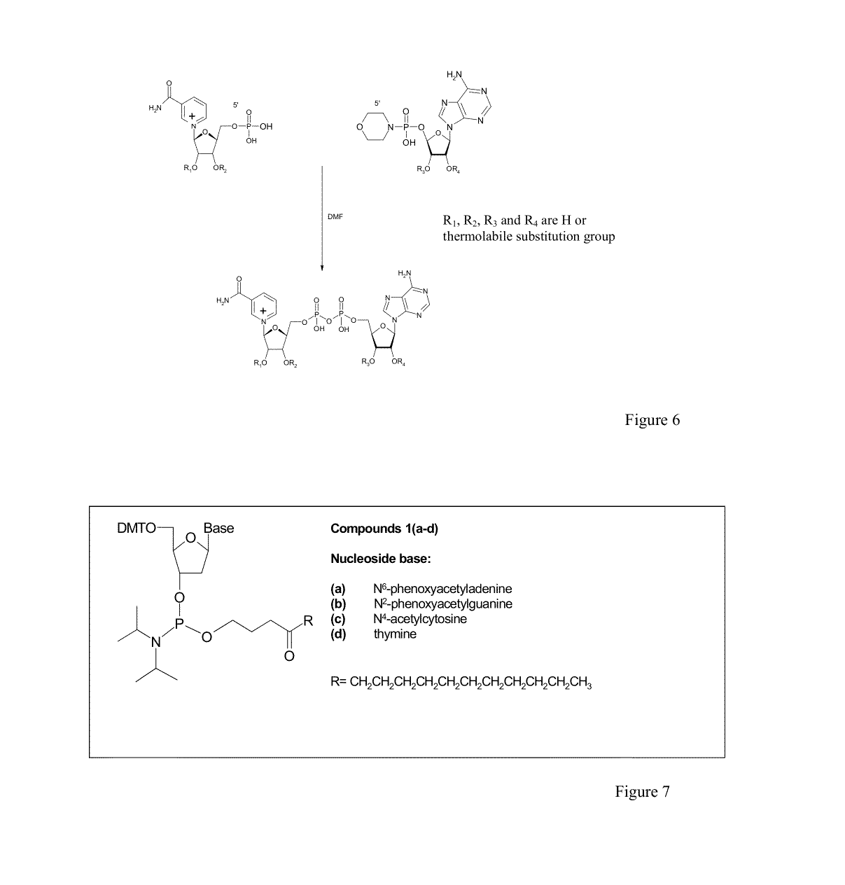

### Route 1 (FIG. 6)

Adenosine 5′-monophosphomorpholidate 4-morpholine-N,N′-dicyclohexylcarboxamidine salt (0.3 mmol) and TBE-NRP (or CHE-NRP) astriethylammonium salt (0.15 mmol) are dissolved in 2 mL of anhydrous DMF and kept at 30° C. for 3 days. The mixture is diluted with 30 mL of 100 mM TEAB, pH 8.5, and P¹-5′-[2′,3′-O-bis-substitutednicotinamide-riboside-P²-5′-adenosine pyphosphate is purified by anion exchange chromatography on column with DEAE Sephadex A25 using gradient of TEAB, pH 8.5. Fractions containing product are combined and carefully concentrated on rotary evaporator at reduced pressure (at temperature below 30° C.). Expected yield is 0.1 mmol, 35%.

### Route 2

Adenosine 5′-monophosphate, pridinium salt (0.2 mmol) is dissolved in 5 mL of methanol containing 1 eq of tri-n-octylamine The mixture is stirred for 3 hours, evaporated on rotary evaporator and co-evaporation with anhydrous pyridine (2×5 mL) and finally with anhydrous DMF (2×5 mL). The residue is dissolved in 1 mL of anhydrous DMF and carbonylimidazole (5 eq, 1.0 mmol) is added. The mixture is stirred for 6 hours. Excess carbonyldiimidazole is quenched by adding 75 uL of methanol and a solution of 2′,3′-O-bis-substitutednicotinamide riboside 5′-phosphate (0.15 mmol) in DMF (4.0 mL) containing tributylamine (0.35 mmol) is added. The reaction is stirred for 3 days at room temperature. The 100 mM TEAB (pH 8.5, 50 mL) is added and stirred for 30 min. P¹-5′-{2′,3′-O-bis-[2-(tert-butoxy)ethyl]}-nicotinamide riboside-P²-5′-adenosine pyphosphate is purified by anion exchange chromatography on column with DEAE Sephadex A25 using gradient of TEAB. Fractions containing product are combined and carefully concentrated on rotary evaporator at reduced pressure (at temperature below 30° C.). Expected yield is 0.1 mmol, 50%.

By following the same procedure but using substituted and/or substituted derivatives of AMP and nicotinamide riboside 5′-phosphate several other substituted NAD+ derivatives mentioned can be synthesized.

### Example 3

### Synthesis of 4-oxotetradecyl (OXT) Substituted Donor and Acceptor Probes and Adenylated Donor Intermediate at 1 μmol Scale: General Synthesis Procedure

Automated solid-phase synthesis of OXT-substituted oligonucleotide starts from an appropriate dT, dC, dA or dG CPG-support-filled column (1 μmol scale, Glen Research) using fast deprotecting 2-cyanoethyl phosphoramidite monomers of dA, dT, dC and dG (Glen Research) and manufacture recommended synthesis protocol. The OXT group is introduced at specific internucleotide position the oligonucleotide chain using the appropriate OXT phosphoramidite (FIG. 7) and synthesis is continued with 2-cyanoethyl phosphoramidites.

Allow 10 min for each OXT phosphoramidite coupling. The appropriate 2′-deoxynucleoside OXT-phosphoramidite (Glen Research) is dissolved in anhydrous acetonitrile at 0.1 M concentration, activated molecular sieves type 3 Å (0.1 g/mL of solution) are added and the mixture is stored overnight at room temperature under argon atmosphere before use.

After completion of the oligonucleotide synthesis the 5′-DMT group is removed after the last coupling/oxidation step on the synthesizer. The column is washed with acetonitrile (e.g., two times with 2 mL), and dried using argon gas flow for 5-10 min.

### OXT-SAP

For OXT-SAP proceed to deprotection/cleavage and HPLC isolation steps (see below).

### OXT-SDP and OXT-SADI

### 5′-Phosphitylation of Donor Probe on Solid Support Using Diphenyl Phosphite (Adapted from Zlatev et al. Organic Letters 12, 2190-2193 (2010))

2 mL of a 1 M solution of diphenyl phosphite (Aldrich) in anhydrous pyridine (Aldrich) is pushed manually through the 1.0 μmol column of the solid-supported 5′-OH OXT-substituted oligonucleotide using two syringes; the solution is pushed back and forth for 5 minutes at room temperature and then left to react for 30 minutes. The solution is pushed out; the column is washed with anhydrous acetonitrile (3 mL; Glen Research) and dried under a stream of argon. 2 mL of 100 mM aqueous TEAB (pH=8.0, Aldrich) is pushed back and forth through the column for 5 min using two syringes and is then left to react for 2 hours. The solution is pushed out; the column is washed twice with 5 mL of anhydrous acetonitrile and reverse-flushed with argon. It is placed under vacuum over P₂O₅ for 24 hours, and is then stored at −20° C.

The conversion rate of the oligonucleotide to its 5′ H-phosphonate monoester, using this method, is above 95%. The reaction is monitored by HPLC and MALDI/TOF MS after deprotection of an aliquot of the beads (using 50 mM potassium carbonate in methanol for 20 hrs at room temperature).

### Preparation of 5′-phosphoroimidazolidate Oligonucleotide on Support Using Amidative oxidation

Activated 4 Å molecular sieves (3 to 5 beads) are added to the solid-supported 5′-H-phosphonate oligonucleotide (1.0 μmol) in a synthesis column (see previous step). The column is closed and flushed with argon. The oxidation solution is then prepared as follows: 150 mg (2 mmol) of imidazole (Aldrich) are co-evaporated twice with anhydrous acetonitrile and then dried under vacuum over P₂O₅. The residue is re-dissolved in anhydrous acetonitrile (Glen Research, 0.8 mL), anhydrous carbon tetrachloride (Aldrich, 0.8 mL), anhydrous triethylamine (Sigma, 0.1 mL), and N,O-bis-trimethylsilyl acetamide (Aldrich, 0.4 mL). The resulting solution is dried over activated 4 Å molecular sieves for 1 hr, and then degassed with dry argon for 30 seconds. The solution is pushed back and forth through the above column containing the solid-supported 5′-H-phosphonate oligonucleotides using two syringes and then allowed to react for 5 hours at room temperature. The solution is removed from the column and the support is washed twice with methanol, then reverse-flushed with argon.

### Preparation of OXT-SDP Using 5′-phosphoroimidazolidate Oligonucleotide on Support

A mixture of pyridine-water (1:1, v/v; 1 mL) is pushed back and forth for 5 min through the column containing 1-μmol of the solid-supported 5′-phosphoroimidazolidate oligonucleotide using two syringes and then is allowed to react for 1 h at room temperature. The solution is removed and the support is washed with methanol (2×5 mL) and acetonitrile (2×5 mL), followed by a flush with argon. For completion of the preparation of OXT-SDP proceed to deprotection/cleavage and HPLC isolation steps (below).

### Preparation of OXT-SADI Using AMP and its Derivatives

0.25 M solution of substituted or unsubstituted AMP (tri-octylammonium salt) in anhydrous DMF (Aldrich) is prepared and stored over activated 3 Å molecular sieves for 2 days at 4° C. 1 mL of that solution is added to the solid-supported 5′-phosphorylated donor oligonucleotide in a 1-μmol column. The solution is pushed back and forth through the synthesis column for 5 min, then left to react for 24 h at room temperature. The solution is removed and the support is washed with methanol (2×5 mL) and acetonitrile (2×5 mL), followed by a flush with argon. For completion of preparation of OXT-SADI proceed to deprotection/cleavage and HPLC isolation steps (below).

Deprotection and Cleavage of OXT-Substituted Oligonucleotide from Solid Support

The solid support is transferred from column into 8 mL screw-capped vial and 6.0 mL of 50 mM potassium carbonate in methanol is added. The vial is placed on rotary mixer (2-4 rpm) for 16-20 hr at room temperature. The solution is transferred to a new screw cap 8 mL vial and support is washed with 2.0 mL of 1 M TEAA. The washes are combined with a supernatant solution. The solution is cooled for 20 min in freezer at −80° C. (or for 40 min at −20° C.) and the cold vial is placed in Speedvac concentrator for 1-2 hr at high vacuum to reduce the total volume to 1-2 mL by removing most of the methanol. A typical crude yield for synthesis of 25-30 mer OXT-oligonucleotide is 250 O.D. units for 1.0 μmol scale.

### Reverse Phase HPLC Isolation and SepPak Desalting of OXT-Substituted Oligonucleotide

HPLC system with C₁₈ reverse phase DeltaPak column (19×300 mm) is used. The column is washed with 100 mL of acetonitrile and equilibrated with 250 mL of 50 mM TEAB, pH 8.5; flow rate 9 mL/min. Sample from deprotection procedure (see previous steps above) is diluted with acetonitrile to an appropriate volume, if necessary. Injections of sample volumes of 5 mL or less are recommended for preparative HPLC. The gradient of acetonitrile (0-100% over 80 min) in 50 mM TEAB is used; flow rate 9 mL/min.

Retention time for OXT oligonucleotide depends on the primary structure and length of the oligonucleotide. A partial loss of one or two OXT groups from single or double OXT substituted oligonucleotide is observed after this deprotection procedure.

Appropriate fractions containing pure OXT-substituted oligonucleotide are combined and concentrated in Speedvac concentrator to approximately 5 mL (to remove most of the acetonitrile). The concentrated fractions are diluted with 5× volume of 50 mM TEAB, pH 8.5 and desalted immediately.

Sample is applied to SepPak C₁₈ cartridge with a flow rate of 2 mL/min. The cartridge is rinsed with 10 mL of 50 mM TEAB, pH 8.5 over 2 min followed by 20 mL of water over 2 min. 1 mL of 50% acetonitrile in water (v/v) is placed in 1 mL syringe and this solution is pushed through cartridge (1-2 drop/sec) collecting ˜100 μL fractions while keeping fractions on ice. Concentration of OXT-oligonucleotide in collected fractions is determined by UV measurement at 260 nm. Appropriate fractions are combined, transferred into 1.5 mL plastic tubes (˜100-200 μL per tube) and placed at −80° C. in freezer for 15 min or at −20° C. for 40 min.

The cold tubes are placed in Speedvac concentrator and concentrated under high vacuum to remove acetonitrile (0.5 mm of Hg or lower is recommended). Typically, this step takes 30 min or less to bring the final volume in tube to 20-40 μL. If needed, the concentration of OXT oligonucleotide is adjusted with addition of water. Recommended concentration of OXT-substituted oligonucleotide is 250 μM.

Control and maintain temperature in Speedvac concentrator below 35° C. during evaporation. Do not dry out sample. A high level of precaution is recommended during this procedure since OXT primers are not stable in aqueous media at room temperature.

### Example 4

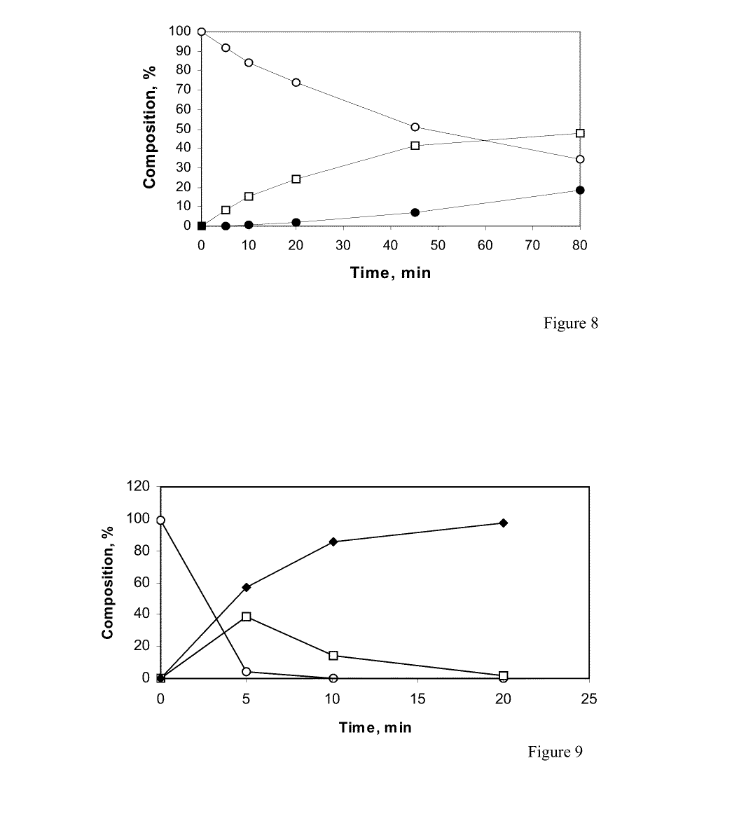

### Kinetics of Thermal Conversion of THF- and TBE-Substituted ATP Cofactors to Unsubstituted ATP (FIGS. 8 and 9)

Conversion of the 2′,3′-substituted ATP to the corresponding unsubstituted ATP was investigated in ligase buffer (pH 7.5, Table 1) at 95° C. The reactions were monitored by analysis of the aliquot of incubated mixture by reverse-phase and anion-exchange HPLC. The resultant graph of formation of ATP versus time is presented in FIGS. 8 and 9. The estimated concentration of the ATP that formed from 2′,3′-substituted ATP after 5, 10, 20, 45 and 80 min of incubation at 95° C. are presented in Table 1.

TABLE 1
Estimated concentration of unsubstituted ATP forming in 1 mM solution
of 2′,3′-substituted ATP during incubation at 95° C. in ligase
buffer (50 mM Tris-HCl, 10 mM MgCl₂, 10 mM dithiothreitol,
25 μg/ml bovine serum albumin (at 25° C. buffer pH is 7.5).

| 2′,3′-substitution | Concentration of unsubstituted ATP, μM | | | | |
|---|---|---|---|---|---|
| group | 5 min | 10 min | 20 min | 45 min | 80 min |
| —O(TBE) | 569 | 857 | 970 | — | — |
| —O(THF) | 2.8 | 4.6 | 17.3 | 72.7 | 182.2 |

### Example 5

### Kinetics of Thermal Conversion of OXT-SAP to the Corresponding Unsubstituted Acceptor Probe

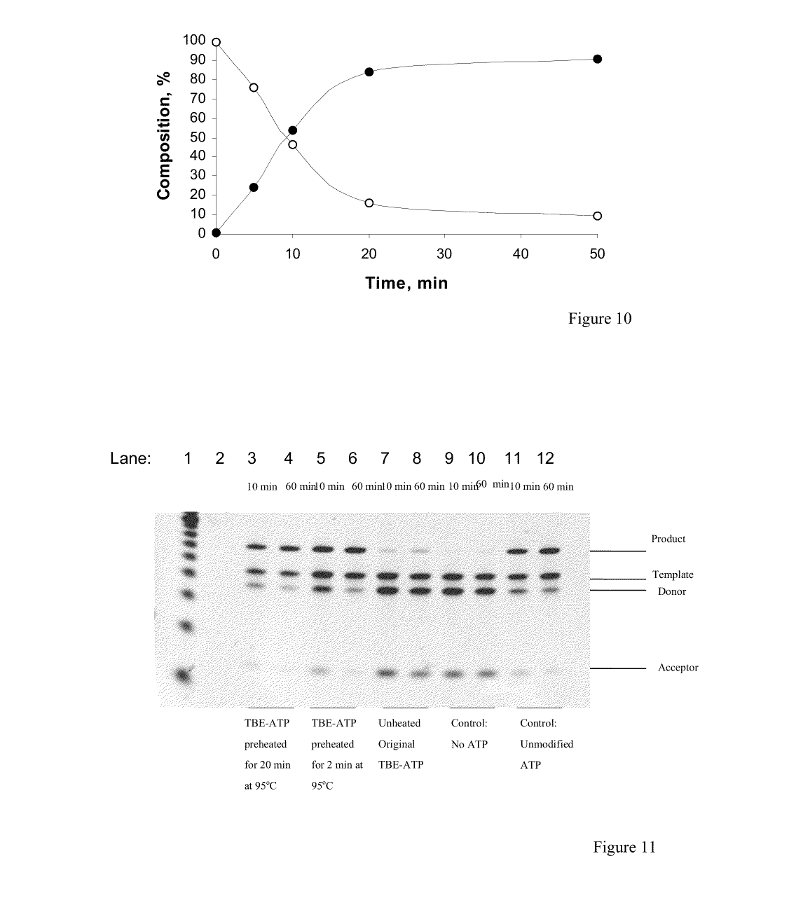

Conversion of the OXT-SAP to corresponding unsubstituted probe was investigated in ligase buffer (pH 7.5, Table 1) at 95° C. The reactions were monitored by analysis of the aliquot of incubated mixture by reverse-phase HPLC. The resultant graph of formation of unsubstituted probe versus time is presented in FIG. 10. The estimated concentration of the unsubstituted probe formed from OXT-substituted probe after 5, 10, 20, 40 and 80 minutes of incubation at 95° C. are presented in Table 2.

TABLE 2
Estimated concentration of unsubstituted acceptor probe forming in
1.0 μM solution of OXT-substituted probe during incubation at 95° C. in
ligase buffer (50 mM Tris-HCl, 10 mM MgCl₂, 10 mM dithiothreitol,
25 μg/ml bovine serum albumin (at 25° C. buffer pH is 7.5).

| | |
|---|---|
| | Concentration of unsubstituted |
| Substitution | acceptor probe, μM |

| group | 5 min | 10 min | 20 min | 50 min |
|---|---|---|---|---|
| OXT | 0.25 | 0.58 | 0.84 | 0.88 |

TABLE 3
Sequences of OXT-substututed and unsubstituted acceptor and
donor probes and matched and mismatched DNA targets used in
Examples 5-7 (all oligonucleotides are 2′-deoxyribo series;
OXT: 4-oxo-tetradecyl phosphotriester internucleotide linkage)

| Acceptor probe | Sequence (5′ > 3′) |
|---|---|
| OXT-SAP | CCCTGTTCCAGCGTCGGTGTTGCGT(OXT)T |
| Unsubstituted acceptor probe | CCCTGTTCCAGCGTCGGTGTTGCGTT |
| DNA Target | Sequence (3′ > 5′) |
| Matched target (A-T) | GGGACAAGGTCGCAGCCACAACGCAATCAACAGTAT |
| | CAAACTAGGAGATCAGACCC |
| Mismatched target (G-T) | GGGACAAGGTCGCAGCCACAACGCAGTCAACAGTAT |
| | CAAACTAGGAGATCAGACCC |
| Donor probe | Sequence (5′ > 3′) |
| Unsubstituted donor probe | pAGTTGTCATAGTTTGATCCTCTAGTCTGGGAGTATT |
| | CTAGGCGACTGGTAC |

### Example 6

### Detection of Ligase Reaction with Unsubstituted Probes and 2′,3′-Bis-TBE Substituted ATP Cofactor Using T4 DNA Ligase and PAGE Analysis

The ability of T4 DNA ligase to join an unsubstituted acceptor and donor probes was assessed on a complementary template (see Table 3 for the sequences employed) in the presence of 2′,3′-TBE-substituted ATP cofactor. Each 20 μL reaction was performed in ligase buffer containing 50 mM Tris-HCl (pH 7.5), 10 mM MgCl₂, 10 mM dithiothreitol, 25 μg/ml bovine serum albumin and 1 mM 2′,3′-TBE-substituted ATP or unsubstituted ATP in control experiment. The unsubstituted donor and acceptor probes and DNA template were at equimolar 0.1 μM concentration. To prevent premature uncontrollable cleavage of TBE groups from 2′,3′-TBE-substituted ATP, the preparation of the ligation mixture was performed in three steps.

First, a ternary template-acceptor-donor complex was prepared by mixing the template, acceptor and donor strands in ligation buffer, heating the mixture at 95° C. for 2 min, slowly cooling to 4° C. over 15 min, and keeping mixture at 4° C. for 1 hour.

Second, three “thermally treated” solutions 2′,3′-TBE-substituted ATP in ligation buffer were prepared. The first and second solutions were prepared by incubating 2′,3′-TBE-substituted ATP at 95° C. for 2 and 20 min, respectively, to partially convert 2′,3′-TBE-substituted ATP to unmodified ATP. As it follows from the kinetic data (FIG. 9, Table 1), after 2 min about 20% of 2′,3′-TBE-substituted ATP cofactor molecules were converted to the unmodified ATP, while a 20 min incubation resulted in more than 85% conversion of 2′,3′-TBE-substituted ATP to unmodified ATP. The third, control solution, was not incubated at 95° C. and therefore it contained original 2′,3′-TBE-substituted ATP with traces of unmodified ATP (0.1%).

At the third step, solution of the “thermally treated” 2′,3′-TBE-substituted ATP or control solution was added to the ternary oligonucleotide complex at 20° C. Ligation was initiated by adding 10 units of T4 DNA ligase (New England Biolabs) to each reaction mixture. Ligation proceeded at room temperature for 10 or 60 min and was terminated by adding 2×TBE-Urea inactivation buffer (Invitrogen) and heating the reaction to 65° C. for 10 minutes. The reactions were analysed on 6% TBE-Urea Novex polyacrylamide gel (Invitrogen). The products were visualized by staining gels with SYBR Gold nucleic acid stain (Invitrogen) according to manufacturer's protocol (FIG. 11).

An ample formation of 80(+)-mer ligation product was observed in experiments that employed control unsubstituted ATP or “thermally treated” sample of 2′,3′-TBE-substituted ATP. On the contrary, only traces of ligation product were detected for “thermally untreated” (original) 2′,3′-TBE-substituted ATP and for control mixture that did not contain added ATP cofactor.

### Example 7

### Detection of Ligase Reaction with OXT-SAP and Unsubstituted NAD+ Cofactor Using Thermophilic DNA Ligase and PAGE Analysis

The ability of Tth DNA ligase to join an OXT-substituted acceptor and donor probes was assessed on a matched and mismatched DNA template in the presence of NAD+ cofactor (see Table 3 for the sequences employed). Each 20 μL reaction was performed in ligase buffer containing 50 mM Tris-HCl (pH 7.5), 10 mM MgCl₂, 10 mM dithiothreitol, 25 μg/ml bovine serum albumin and 1 mM NAD+. The OXT-SAP, unsubstituted donor probe and DNA template were at equimolar 0.1 μM concentration. To prevent premature uncontrollable cleavage of the OXT groups from OXT-SAP, the preparation of the ligation mixture was performed in three steps.

First, a binary template-donor complex was prepared by mixing the DNA template and donor strands in ligation buffer, heating the mixture at 95° C. for 2 min, slowly cooling to 4° C. over 15 min, and keeping mixture at 4° C. for 1 hour.

Second, the OXT-SAP was added to the mixture and allowed to stay at 4° C. for 12 hours.

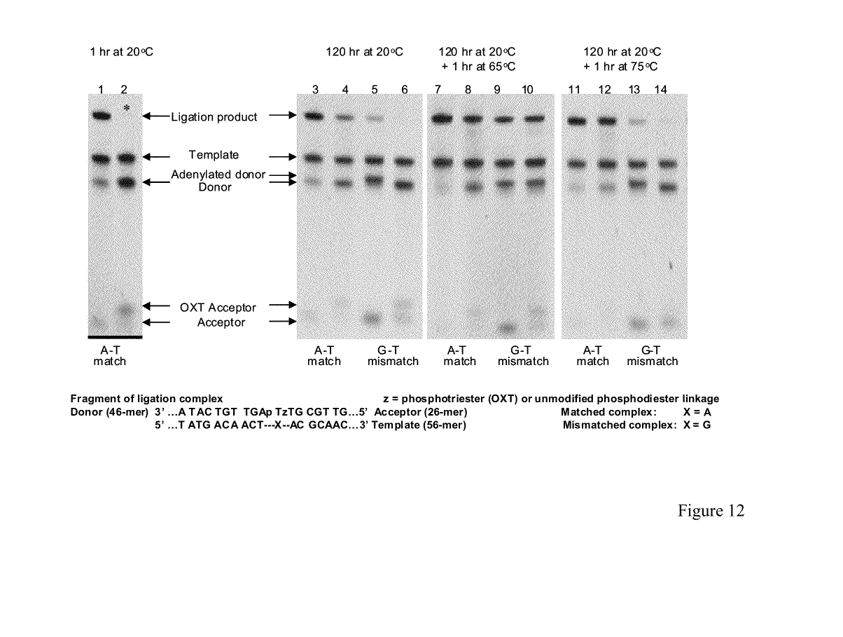

Third, a solutions of NAD+ in ligation buffer was added and ligation was initiated by adding 2 units of Tth DNA ligase to reaction mixture. Ligation proceeded at room temperature for 1 hr and analyzed by PAGE on TBE-Urea denaturing 15% gel (FIG. 12).

Ligation on matched template with unsubstituted acceptor probe resulted in efficient formation of ligation product. On the contrary, no ligation product formed after 1 hr in ligation reaction with OXT-SAP and only small amount of product was detected after 120 hrs.

On mismatched DNA template after 120 hrs of incubation a small amount of ligation product was formed with unsubstituted acceptor probe but no product was detected with OXT-SAP. When this ligation mixture was additionally incubated for 1 h at 75° C. the substantial accumulation of ligation product was detected only for unsubstituted acceptor probe. On the contrary, for matched template and both unsubstituted and OXT-SAPs the ample formation of ligation product was observed.

### Example 8

### General Procedure for HS LCR Using Thermophilic DNA Ligase and Substituted Probe, or SADI, or SC

HS LCR with OXT-Substituted Probes

The reaction mixture (50 uL) is prepared on ice by mixing 10 uL of 5×LCR buffer {250 mM Tris-HCl (pH 7.5), 50 mM MgCl₂, 5 mM dithiothreitol, 125 μg/ml bovine serum albumin}, 5 uL of 10 mM cofactor (ATP or NAD+), 5 uL of 10 uM of each OXT-substituted or unsubstituted probes, 5 uL of DNA target (variable copy number), 15 uL of water and 5 uL (400 U/mL; 2 U total) of thermostable DNA ligase. The reaction mixture is overlaid with oil, and the reaction is activated by placing tube into a thermocycler at 95° C. for 5 min and incubated for 60 cycles consisting of 20 s at 95° C. and 30 s at 55°-65° C. The exact ligation temperature depends on the length and composition of the oligonucleotide probes. The final LCR mixture is analyzed by denaturing PAGE in 7M Urea-TBE at 60-70° C. The bands on the gel are detected by staining with SYBR gold intercalating dye.

HS LCR with SCs

The reaction mixture (50 uL) is prepared on ice by mixing 10 uL of 5×LCR buffer {250 mM Tris-HCl (pH 7.5), 50 mM MgCl₂, 5 mM dithiothreitol, 125 μg/ml bovine serum albumin}, 5 uL of 10 uM of each donor and acceptor probes, 15 uL of water, 5 uL of DNA target (variable copy number), 5 uL (400 U/mL; 2 U total) of thermostable DNA ligase and 5 uL of 10 mM SC (substituted ATP or NAD+). The reaction mixture is overlaid with oil, and reaction is activated by placing tube into a thermocycler at 95° C. for 5 min and incubated for 60 cycles consisting of 20 s at 95° C. and 30 s at 55°-65° C. The exact ligation temperature depends on the length and composition of the oligonucleotide probes. The final LCR mixture is analyzed by denaturing PAGE in 7M Urea-TBE at 60-70° C. The bands on the gel are detected by staining with SYBR gold intercalating dye.

HS LCR with OXT-SADI

The reaction mixture (50 uL) is prepared on ice by mixing 10 uL of 5×LCR buffer {250 mM Tris-HCl (pH 7.5), 50 mM MgCl₂, 5 mM dithiothreitol, 125 μg/ml bovine serum albumin}, 5 uL of 10 uM of acceptor probe, 5 uL of OXT-SADI, 5 uL of DNA target (variable copy number), 20 uL of water and 5 uL (400 U/mL; 2 U total) of thermostable DNA ligase. The reaction mixture is overlaid with oil, and reaction is activated by placing tube into a thermocycler at 95° C. for 5 min and incubated for 60 cycles consisting of 20 s at 95° C. and 30 s at 55°-65° C. The exact ligation temperature depends on the length and composition of the oligonucleotide probes. The final LCR mixture is analyzed by denaturing PAGE in 7M Urea-TBE at 60-70° C. The bands on the gel are detected by staining with SYBR gold intercalating dye.

### Example 9

### Synthesis of Cystic Fibrosis (CF) Oligonucleotides for HS LR and HS LCR (Table 4)

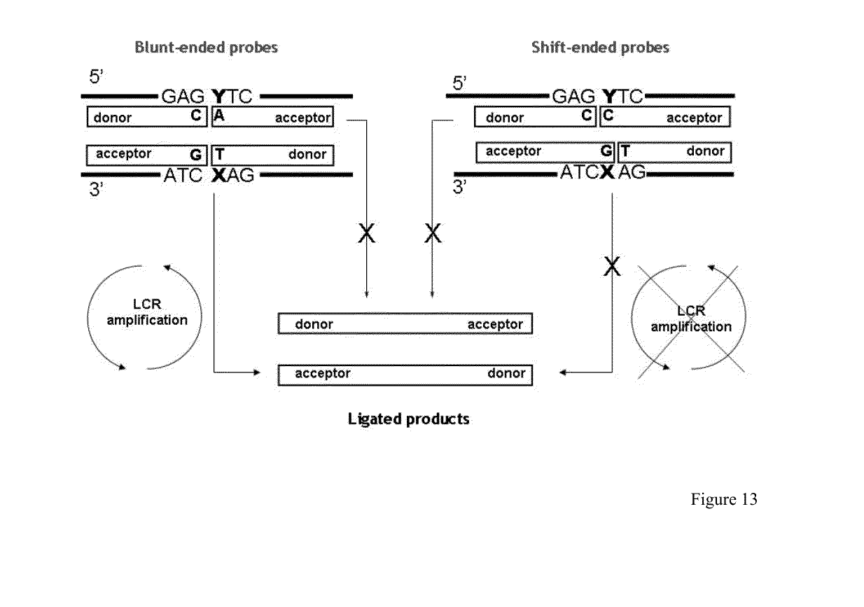

A number of OXT-modified acceptor probes as well as corresponding unmodified control phosphodiester (PDE) probes and donor probes, shown in Table 4, were synthesized according to the published procedure (Lebedev, et al. Nucl. Acids Res. 2008, 36(20): e131) to demonstrate the efficacy of the Hot Start method for conventional ligase reactions and LCRs. All oligonucleotide sequences were designed for detection of cystic fibrosis (CF) mutation G₅₅₁D (Fang, P. et al. Hum. Mutat. 1995, 6:144-151). Donor and acceptor probes were designed in two geometries: blunt-ended (probes 1, 2, 8, 9, 12, 13, 14, 15 and 16) and shift-ended (probes 3, 4, 5 and 7) for comparison in LCR (Table 4 and FIG. 13). Blunt-ended probes were used to compare the present results with those obtained by Fang et al.

### Example 10

### PAGE Analysis of Conventional Ligase Reaction Mixtures with Matched and Mismatched Synthetic CF DNA Templates

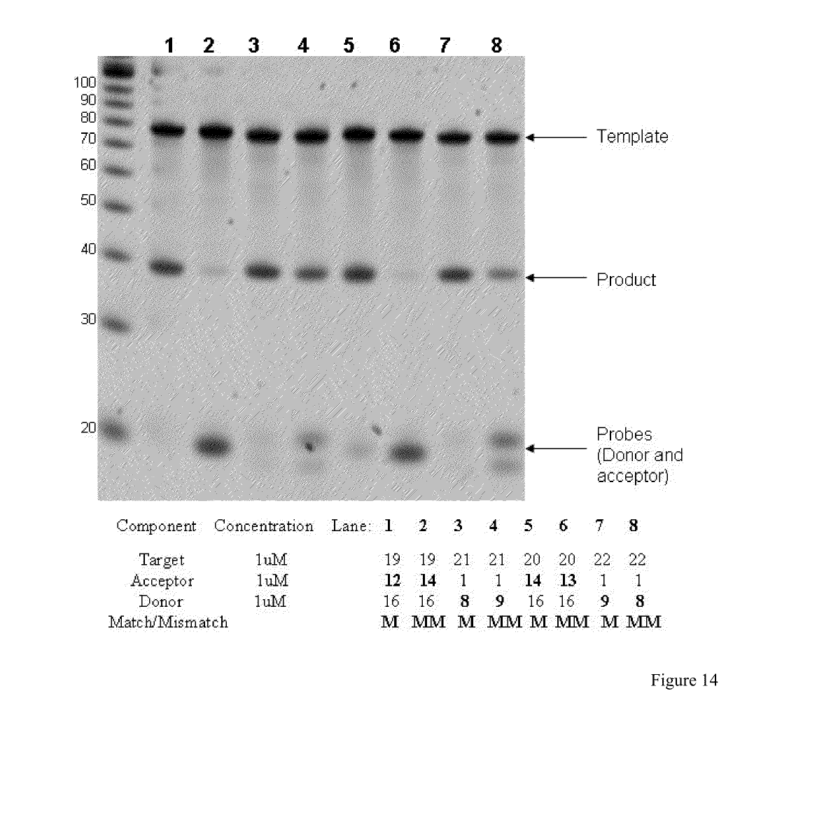

A PAGE analysis was performed on conventional ligase reaction mixtures with matched and mismatched synthetic cystic fibrosis DNA templates. It is known that enzymes exhibit greater discrimination on the 3′ end of the ligation nick in comparison with 5′-side of the nick. This notion was corroborated with the CF G₅₅₁D model system using standard DNA ligase reaction conditions (FIG. 14). The ability of Taq DNA ligase to join unsubstituted (PDE) acceptors and donor probes on wild type template and CF G₅₅₁D mutant template was assessed using equimolar ratios of acceptor:donor:template. Approximately, 1 μM of each acceptor, donor and template oligonucleotides were combined with 40 U Taq DNA ligase in a 12 μL volume and heated for 1 hour at 60° C. The ligation was performed on both templates with corresponding matched and mismatched probes. All probes (1+9), (1+8), (13+16) and (14+16) producted ligated products on matched (M) templates (19, 20, 21 and 22). The probes (1+9) and (1+8) directed against the 5′-end of the donor probe also produced ligated product on mismatched (MM) templates (lanes 4 and 8, respectively), while probes (14+16) and (13+16), directed against the 3′-end of the acceptor probe, produced significantly lower amounts of ligated product on mismatched templates (lanes 2 and 6, respectively).

TABLE 4
Sequences of acceptor and donor probes for CF G₅₅₁D system. OXT- modified
probe (the nucleosides between which the OXT-modified internucleotide
linkage is positioned are underlined); PDE - unmodified phosphodiester
probe; WT - wild type DNA template; G₅₅₁D - mutant DNA template.

| G551D mutated CF DNA system | | |
|---|---|---|
| 16a | OXT Donor | 3′-AC CTT AGT GTG ACT CAC CTCp |
| 15 | OXT Acceptor | TAG T⁻G CTC GTT CTT AAA G-5′ |
| 16 | PDE Donor | 3′-AC CTT AGT GTG ACT CAC CTCp |
| 14 | PDE Acceptor | TAG T⁻G CTC GTT CTT AAA G-5′ |
| 20 | G551D, Sense strand | 5′-ATA TAG TTC TTG GAG AAG GTG GAA TCA |
| | | CAC ⁻GA GTG GAG ATC AAC GAG CAA GAA |
| | | TTT CTT TAG CAA GGT GAA TAA CT |
| 22 | G551D, Antisense | 3′-TAT ATC AAG AAC CTC TTC CAC CTT AGT |
| | strand | GTG ACT CAC CTC TAG TTG CTC GTT CTT |
| | | AAA GAA ATC G⁻T CCA CTT ATT GA |
| 1 | PDE Acceptor | 5′-TG GAA TCA CAC TGA GTG GAG |
| 9 | PDE Donor | pATC AAC GAG CAA GAA TTT C-3′ |
| 2 | OXT Acceptor | 5′-TG GAA TCA CAC TGA GTG GAG |
| 7 | Shifted PDE Donor | pTC AAC GAG CAA GAA TTT C-3′ |
| 5 | Shifted PDE Acceptor | 5′-TG GAA TCA CAC TGA GTG GAG A |
| 6 | Shifted OXT Acceptor | 5′-TG GAA TCA CAC TGA GTG GAG A |
| 9a | OXT Donor | pATC AAC GAG CAA GAA TT⁻ C-3′ |
| 7a | Shifted OXT Donor | pTC AAC GAG CAA GAA TTT C-3′ |
| Wild Type DNA system | | |
| 16a | OXT Donor | 3′-AC CT⁻ AGT GTG ACT CAC CTCp |
| 13a | 2OXT Acceptor | CAG TTG CTC GTT CTT AAA G-5′ |
| 13 | OXT Acceptor | CAG TTG CTC GTT CTT AAA G-5′ |
| 16 | PDE Donor | 3′-AC CT⁻ AGT GTG ACT CAC CTCp |
| 12 | PDE Acceptor | CAG TTG CTC GTT CTT AAA G-5′ |
| 19 | WT, Sense strand | 5-ATA TAG TTC TTG GAG AAG GTG GAA TCA |
| | | CAC TGA GTG GAG GTC AAC GAG CAA GAA |
| | | TTT CTT TAG CAA GGT GAA TAA CT |
| 21 | WT, Antisense strand | 3′-TAT ATC AAG AAC CTC TTC CAC CTT AGT |
| | | GTG ACT CAC CTC CAG TTG CTC GTT CTT |
| | | AAA GAA ATC G⁻T CCA CTT ATT GA |
| 1 | PDE Acceptor | 5′-TG GAA TCA CAC TGA GTG GAG |
| 8 | PDE Donor | pGTC AAC GAG CAA GAA TTT C-3′ |
| 2 | OXT Acceptor | 5′-TG GAA TCA CAC TGA GTG GAG |
| 2a | 2OXT Acceptor | 5′-TG GAA TCA CAC TGA GTG GAG |
| 3 | Shifted PDE Acceptor | 5′-TG GAA TCA CAC TGA GTG GAG G |
| 4 | Shifted OXT Acceptor | 5′-TG GAA TCA CAC TGA GTG GAG G |
| 7 | Shifted PDE Donor | pTC AAC GAG CAA GAA TTT C-3′ |
| 7a | Shifted OXT Donor | pTC AAC GAG CAA GAA TTT C-3′ |
| 8a | OXT Donor | pGTC AAC GAG CAA GAA TTT C-3′ |

### Example 11

### General Procedure for Hot Start Real-Time LCR Using Thermophilic DNA Ligase and OXT Substituted Acceptor Probe, and/or TBE Substituted Cofactor with SYBR® Green Detection

General Temperature Cycling Protocol for Hot Start Real-Time LCR with Substituted Probes and/or Cofactors and Product Detection

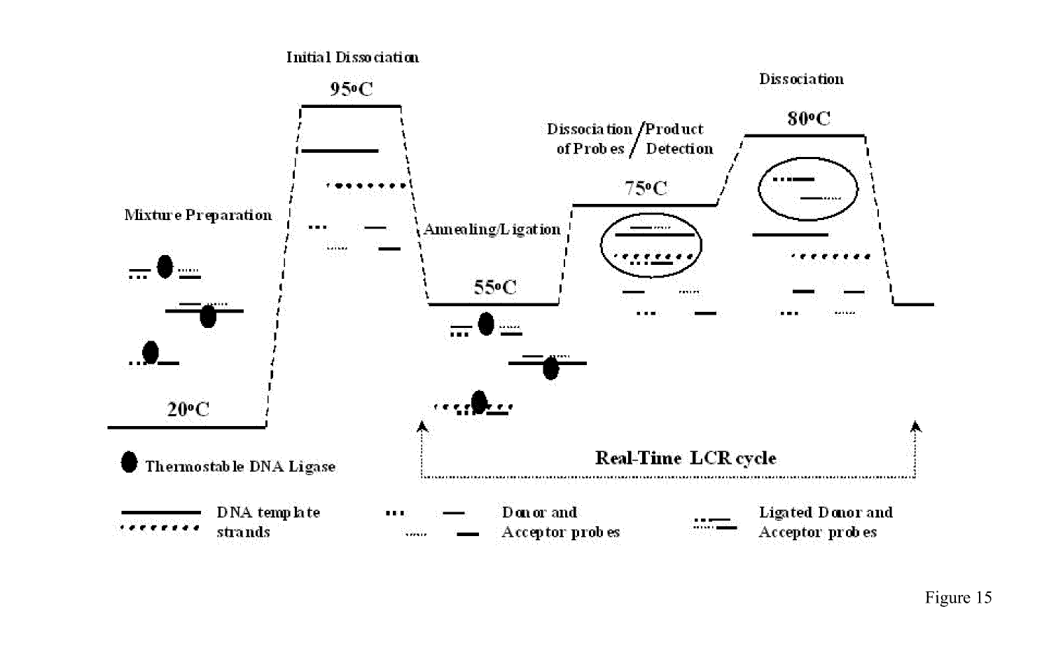

One example of a typical temperature cycling protocol for Real-Time LCR with Hot Start activation utilizing substituted probes and cofactors is shown in FIG. 15. The actual temperatures and length of time for each step will depend on the particular LCR being performed and can be routinely optimized by one skilled in art. A “Product Detection” step is performed within Real-Time LCR procedure to detect and quantify the ligated product. During this step, SYBR® Green dye (Invitrogen, Inc. San Diego, Calif.) intercalates into the double stranded ligation product resulting in a fluorescent signal that is proportional to the product's concentration.

General Hot Start Real-Time LCR with OXT-Substituted Acceptor Probes and Unsubstituted Cofactors

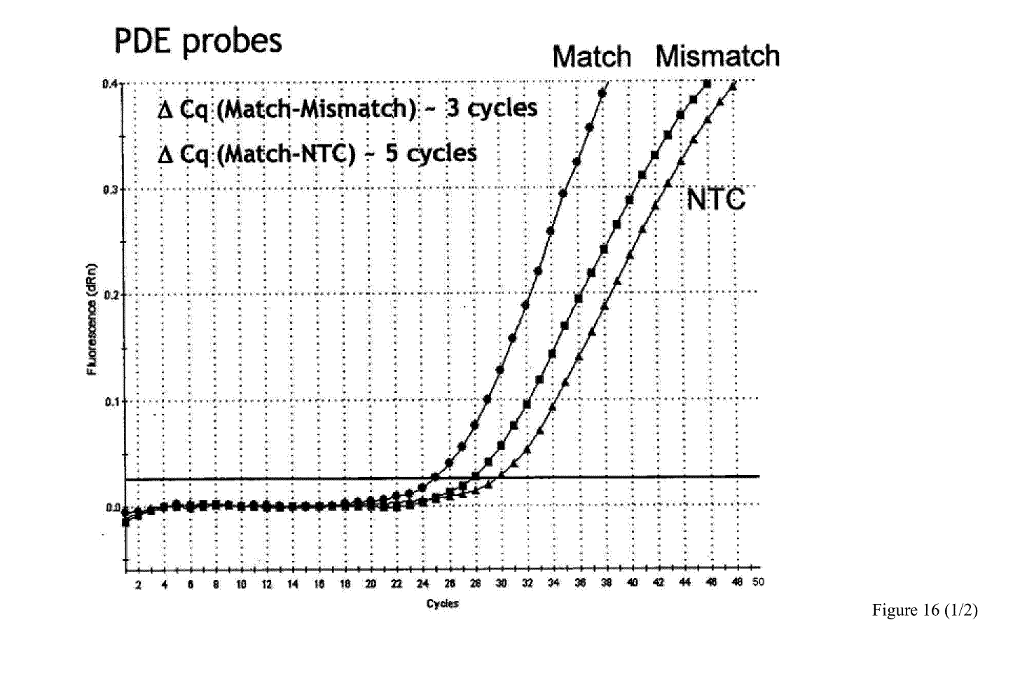

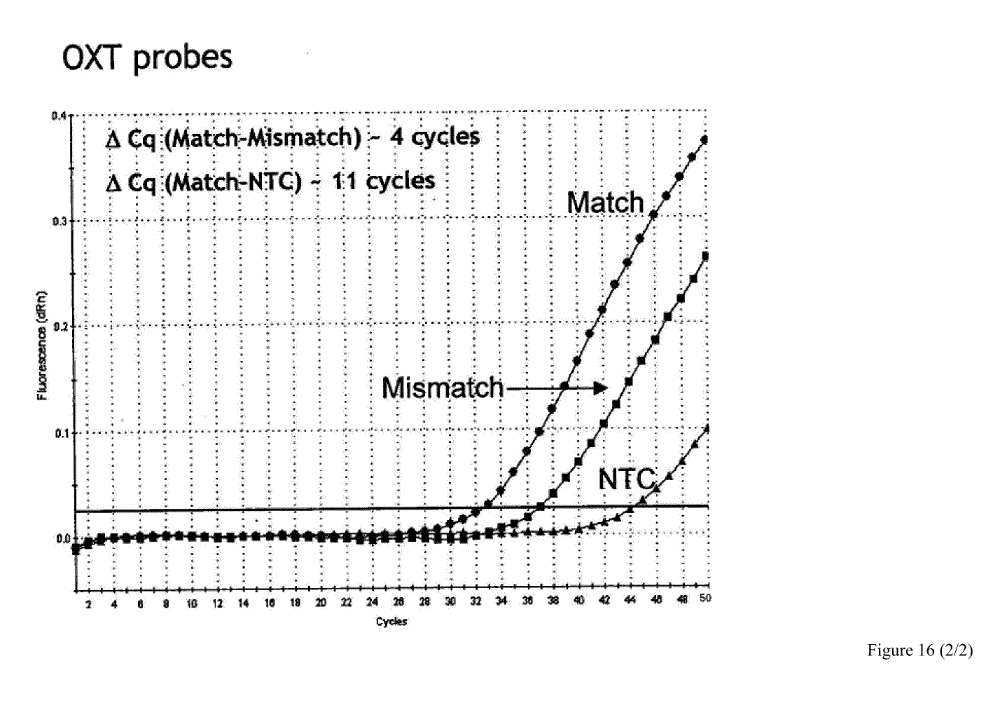

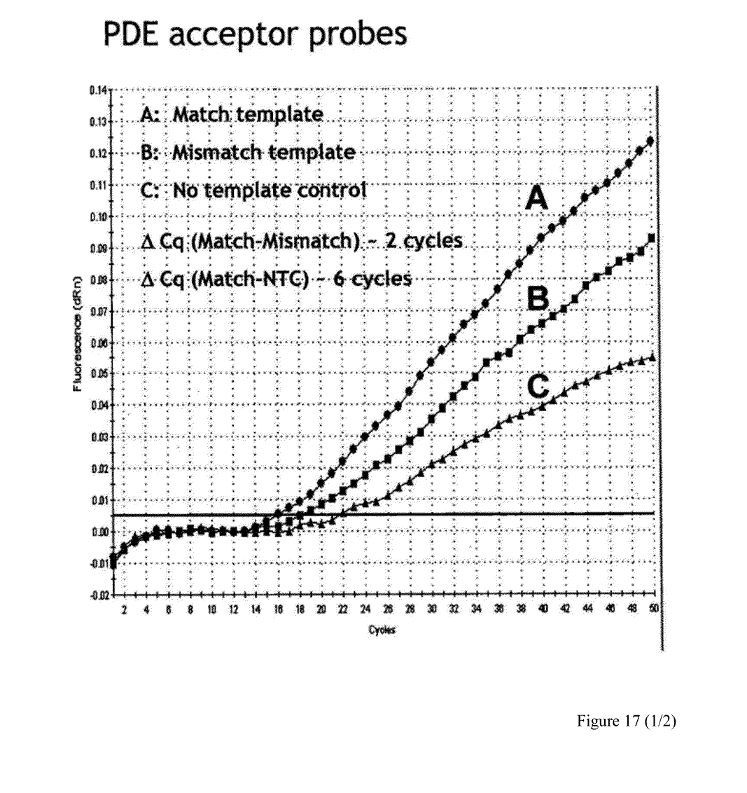

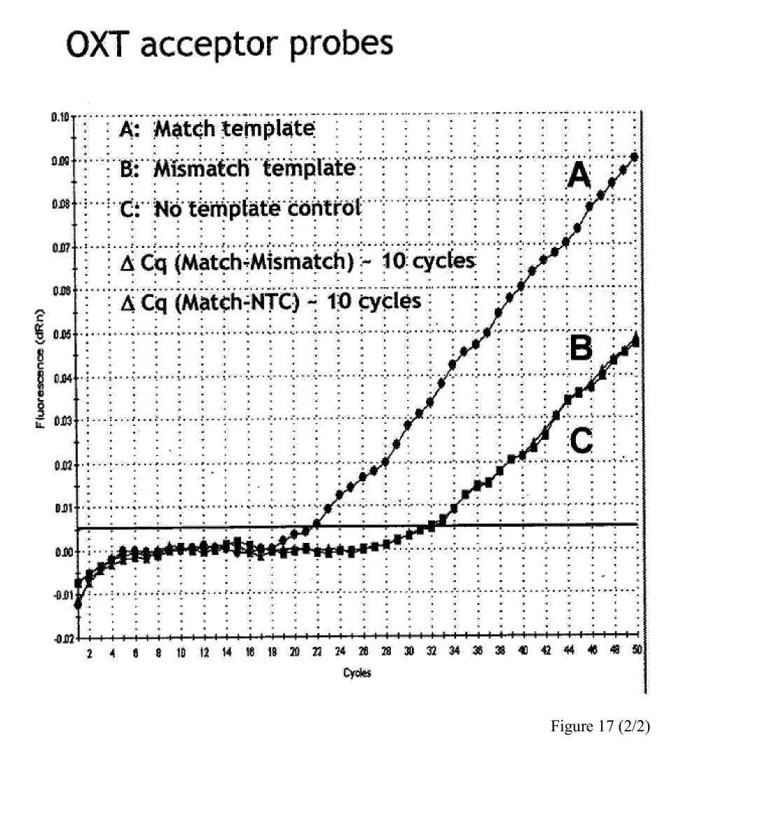

The final reaction mixture (20 μL) contained 0.1 μM of donor and acceptor probes, 1.25× ligase buffer (New England Biolabs, Ipswich, Mass.), synthetic 77-mer dsDNA target (19+21, WT or 20+22 CF G₅₅₁D mutant; variable copy number: from 3×10³ to 3×10⁶ copies and no-template control (NTC)), 5×SYBR® Green dye and 1-20 Units of thermostable DNA ligase. The reaction was activated by placing the reaction mixture into a thermocycler at 95° C. for 2-10 minutes and incubated for 50-70 cycles (consisting of 30 seconds at 95° C., 5-30 seconds at 55°, and 30 seconds at 75° C. (FIG. 15). The actual ligation time at 55° C. varied (see Table 5). Quantitation of the ligation product was performed at 73-75° C. during the “Product Detection” step (FIG. 15). An example of Real-Time LCR using the blunt-ended probe geometry (1+8+12+16, PDE set; 2+8+13+16, OXT set) is shown in FIG. 16. An example of Real-Time LCR using shift-ended probe geometry (3+7+12+16, PDE set; 4+7+13+16, OXT set) is shown in FIG. 17. These examples demonstrate that OXT-substituted acceptor probes substantially improve Real-Time LCR performance by delaying mismatch and NTC ligation.

The amplification curves for the wild type (WT) template, CF G₅₅₁D template, and NTC using PDE probes are relatively close together (see FIG. 16). The differences in Cq values between match/mismatch (M/MM) and M/NTC were 3 and 5 cycles respectively. However, all three amplification curves for the OXT probes were significantly distanced from one other (ΔCq for M/MM was 4 cycles; ΔCq for M/NTC was 11 cycles).

The signals for both the CF G₅₅₁D template and NTC were detected at later cycles than for the WT template when shift-ended PDE probes were used (FIG. 17). Importantly, the curve for mismatched CF G₅₅₁D template coincided with that for NTC, appearing about 10 cycles later than the signal for the matched WT template when shift-ended OXT acceptor probes were used. These findings showed a significant reduction of “false-positive” product formation on the mismatched template by suppressing mismatched ligation in shifted complexes (note: NTC was delayed but not eliminated).

General Hot Start Real-Time LCR with TBE-Substituted Cofactors and PDE Probes

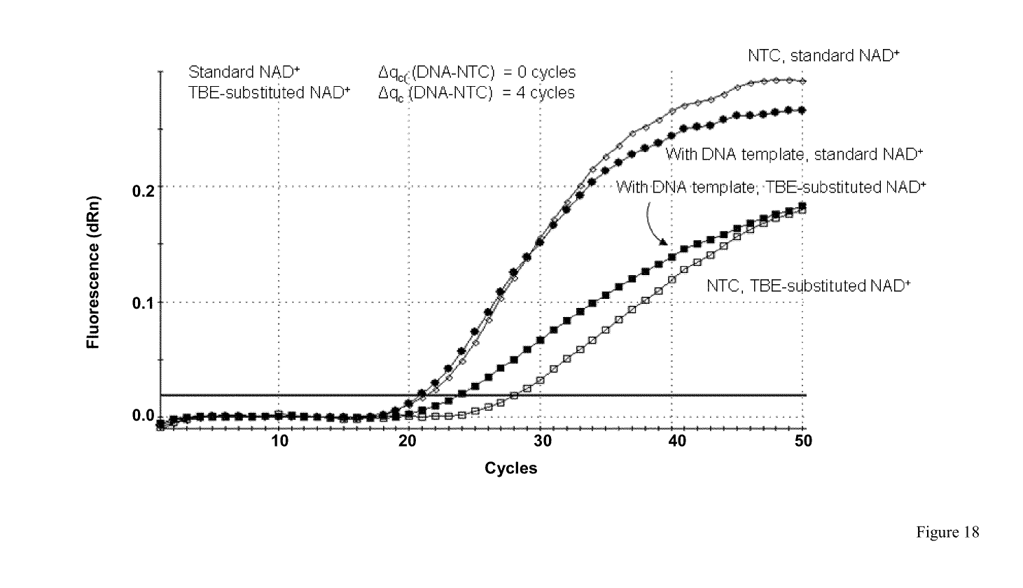

The final reaction mixture (20 μL) contained 0.1 μM of unmodified donor and acceptor probes (1+8+12+16, PDE set), 1.25× ligase buffer (New England Biolabs, Ipswich, Mass.), without cofactor, 1 mM TBE-ATP or TBE-NAD⁺ cofactor, synthetic 77-mer dsDNA target (19+21, WT or 20+22 CF G₅₅₁D mutant; variable copy number: from 3×10³ to 3×10⁶ copies and no-template control (NTC)), 5×SYBR® Green dye and 1-20 Units of thermostable DNA ligase. The reaction was activated by placing the reaction mixture into a thermocycler at 95° C. for 5 minutes and incubated for 50-70 cycles (consisting of 30 seconds at 95° C., 5-30 seconds at 55°, and 30 seconds at 74-75° C., see FIG. 15. An example of Real-Time LCR with TBE-substituted NAD⁺ is shown in FIG. 18. The detection and quantitation of the ligation product was performed at 74° C. during the “Product Detection Step (FIG. 15). The actual ligation time was 5 sec at 55° C. The graph demonstrates that the use of a TBE-substituted NAD⁺ cofactor substantially reduces template independent ligation (TIL).

General Real-Time HS LCR with TBE-Substituted Cofactors, OXT-Substituted Acceptor Probes and PDE Donor Probes.

The final reaction mixture (20 μL) contains 0.1 μM of donor and acceptor probes (2+8+13+16, OXT set), 1× ligase buffer (New England Biolabs, Ipswich, Mass.), without cofactor, 1 mM TBE-ATP or TBE-NAD⁺ cofactor, synthetic 77-mer dsDNA target (19+21, WT or 20+22 CF G₅₅₁D mutant; variable copy number: from 3×10³ to 3×10⁶ copies and NTC), 5×SYBR® Green dye and 1-20 Units of thermostable DNA ligase. The reaction is activated by placing the reaction mixture into a thermocycler at 95° C. for 10 minutes and incubated for 50-70 cycles (consisting of 30 seconds at 95° C., 5-30 seconds at 55°, and 30 seconds at 75° C.) (see FIG. 15). The actual ligation time was varied at 55° C. (Table 5). The detection and quantitation of the ligation product is performed at 75° C. during the “Product Detection Step (FIG. 15).

### Example 12

### Optimizing Hot Start Real-Time Ligase Chain Reaction Parameters Using Thermophilic DNA Ligase and OXT-Substituted Acceptor Probe

The parameters of the Hot Start Real-Time LCR were varied in order to optimize performance conditions so that strict discrimination between match, mismatch and NTC reactions could be achieved. These parameters included cycling times and temperatures (activation, annealing, ligation and detection), concentration and type of enzyme, concentration of probes and cofactor, buffer composition as well as structural features of LCR probes and cofactors. The difference in Cq values between matched and mismatched templates and between matched template and NTC was always greater for blunt-ended OXT-substituted acceptor probes (2+8+13+16) than for blunt-ended PDE acceptor probes (1+8+12+16, see Table 5).

TABLE 5
Dependence of ΔCq values on selected Real-Time LCR parameters for PDE
unmodified (1 + 8 + 12 + 16) and OXT-modified (2 + 8 + 13 + 16) probe sets

| | | Probe | Hot Start | Annealing/ | | 1 = Cq^{NTC} − | | 2 = Cq^{Mismatch} − | |
|---|---|---|---|---|---|---|---|---|---|
| DNA | Units | conc, | Activation | Ligation | | Cq^{Match} | | Cq^{Match} | |
| Ligase | per rxn | μM | time, min | time, sec | temp, ° C. | PDE | OXT | PDE | OXT |
| Taq | 10 | 0.1 | 2 | 30 | 55 | 1.3 | 5.7 | | |
| | 20 | 0.1 | 5 | 30 | 55 | 6.3 | 10.0 | | |
| | 20 | 0.1 | 5 | 30 | 50 | 2.5 | 5.1 | 2.0 | 3.0 |
| | 20 | 0.1 | 5 | 30 | 55 | 4.2 | 7.7 | 2.9 | 4.2 |
| | 20 | 0.1 | 5 | 30 | 60 | 5.5 | 8.6 | 3.7 | 4.6 |
| | 20 | 0.1 | 5 | 30 | 65 | 1.4 | 12.0 | 3.0 | 3.7 |
| | 20 | 0.1 | 5 | 5 | 55 | 2.9 | 7.0 | 2.4 | 5.7 |
| | 20 | 0.1 | 5 | 5 | 60 | 3.3 | 5.4 | 3.6 | 7.4 |
| | 20 | 0.1 | 10 | 30 | 55 | 4.4 | 7.5 (11)* | 1.8 | 2.7 (6.3)* |
| Amp** | 1.25 | 0.1 | 2 | 30 | 55 | 9.5 | 18.3 | | |
| | 2.5 | 0.1 | 2 | 30 | 55 | 0.6 | 8.3 | | |
| | 5 | 0.1 | 2 | 30 | 55 | 2.5 | 5.0 | | |
| | 10 | 0.1 | 2 | 30 | 55 | 2.0 | 4.5 | | |
| | 5 | 0.05 | 2 | 30 | 55 | 11.9 | 15.8 | | |
| | 5 | 0.05 | 5 | 30 | 55 | 15.5 | 22.5 | | |
| | 5 | 0.1 | 2 | 30 | 55 | 6.6 | 8.4 | | |
| | 5 | 0.25 | 2 | 30 | 55 | 2.5 | 4.9 | | |

*Numbers in parentheses are given for doubly OXT substituted acceptor probes 2a and 13a.
**Amp stands for Ampligase DNA Ligase

### Example 13

### Blocking Template Independent Ligation (TIL) with OXT-Substituted Acceptor Probes Prior Hot Start Activation in Real-Time LCR

The ability of OXT-substituted acceptor probes to block TIL prior to Hot Start activation was determined using blunt-ended and shift-ended probes. Each NTC reaction which contained a set of four corresponding LCR probes (either PDE or OXT sets) and Taq DNA ligase was pre-incubated at room temperature for different amounts of time, prior to being placed into the thermocycler. The results are presented in Table 6. The data demonstrates that the longer the PDE probes were incubated with enzyme at room temperature prior to start of LCR, the more efficiently TIL occurred during LCR, resulting in progressively smaller Cq value. However, the Cq value were largely unaffected by the pre-incubation time using OXT-substituted acceptor probes. These results indicate that OXT-substituted acceptor probes suppressed “cold” non-specific ligation prior to the start of LCR significantly reducing template independent formation of “false-positive” products. This effect was even more pronounced when SAPs contained two OXT-groups at the 3′-end (Table 6).

TABLE 6
Dependence of Cq values for no-template controls (NTC) on pre-LCR
incubation time.

| | | Cq | | | | |
|---|---|---|---|---|---|---|
| | | Blunt-ended | | | Shift-ended | |
| Incubation time at RT | | PDE | 1xOXT | 2xOXT | PDE | 1xOXT |
| 15 | min | 22 | 27 | 34 | 22 | 31 |
| 5 | hours | 17 | 26 | 34 | 19 | 30 |

### Example 14

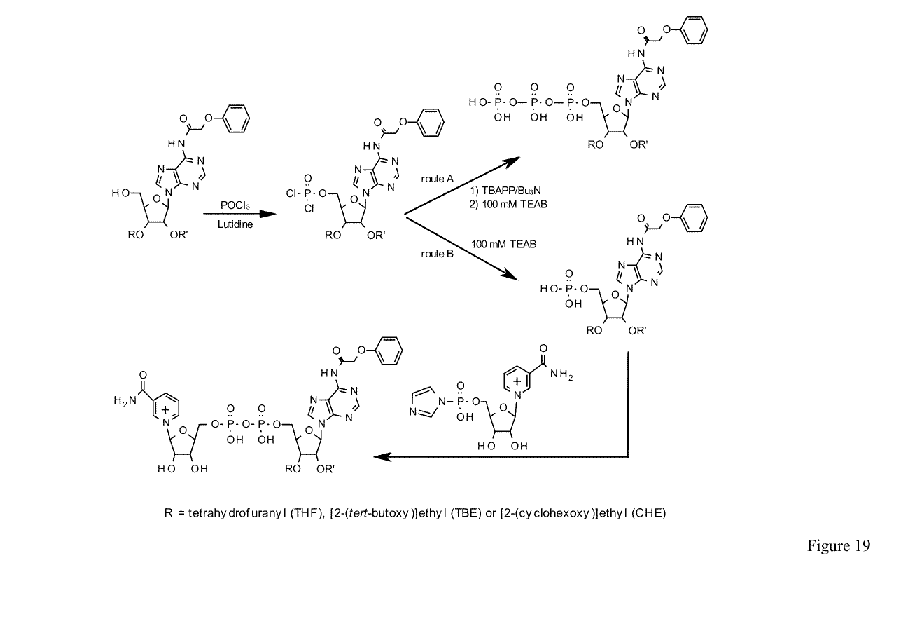

### Synthesis of 2′,3′-O-bis-substituted-N⁶-phenoxyacetyl adenosine-5′-triphosphates (FIG. 19)

### Synthesis of 5′-O-TBDMS-N⁶-phenoxyacetyl-adenosine

Approximately 0.6 g (2.0 mmol) of N⁶-phenoxyacetyl-adenosine (Cat# PM-6001 ChemGenes, Wilmington, Mass.) is co-evaporated with dry pyridine (10 mL), re-dissolved in 5 mL of dry pyridine and treated with 2.0 mmol of dimethyl-tert-butylsilyl chloride (TBDMS-Cl) for 24 hours at room temperature. Saturated aqueous solution of sodium bicarbonate (20 mL) is added and the mixture is extracted twice with 20 mL of dichloromethane. Organic layers are combined, dried over Na₂SO₄, filtered, and evaporated to remove solvents. The residue is dissolved in ethyl acetate and purified on silica gel column (20 g) using 5% methanol in ethyl acetate as eluting solution. Fractions containing 5′-O-TBDMS-N⁶-phenoxyacetyl-adenosine are pooled and dried on rotary evaporator.

### Synthesis of 2′,3′-O-bis-substituted-N⁶-phenoxyacetyl-adenosine

5′-O-TBDMS-N⁶-phenoxyacetyl-adenosine is treated with 5 eqs of 2,3-dihydrofuran (or tert-butyl vinyl ether or cyclohexyl vinyl ether) in the presence of 0.5 eqs of pyridinium p-toluenesulfonate in dioxane for 16 hours at room temperature. The reaction mixture is then evaporated and co-evaporated on rotary evaporator with dichloromethane (30 mL) and toluene (20 mL). The residue is dissolved in dichloromethane (20 mL) and extracted twice with aqueous sodium bicarbonate (20 mL). Organic layers are combined and dried over anhydrous sodium sulfate, filtered and concentrated on rotary evaporator. Subsequent treatment of the residue with 4 mmol of tetra-n-butylammonium fluoride in 10 mL of tetrahydrofurane:pyridine:water (8:1:1, v/v) for two hours at room temperature produces 2′,3′-O-bis-tetrahydrofuranyl (THF) (or 2′,3′-O-bis-[2-(tert-butoxy)]ethyl (TBE) or 2′,3′-O-bis-[2-(cyclohexoxy)]ethyl (CHE) derivatives of N⁶-phenoxyacetyl-adenosine, respectively. The compounds are isolated on a silica gel column using 2% MeOH in CH₂Cl₂ as eluting solution. Fractions containing 2′,3′-O-bis-substituted-N⁶-phonoxyacetyl-adenosine are pooled and dried. Product yield was about 30-50%.

### Synthesis of 2,3′-O-bis-substituted-N⁶-phenoxyacetyl-adenosine-5′-triphosphate

2′,3′-O-bis-substituted N⁶-phenoxyacetyl-adenosine (0.33 mmol) is reacted with 2.0 equivalents of POCl₃ in 1.7 mL of trimethylphosphate:lutidine (5:1, v/v) at 0° C. for 30 minutes followed by reaction with 5 equiv. of tributylammonium pyrophosphate and 4 equiv. of tributylamine. After stirring mixture for 3 minutes at room temperature 30 mL of aqueous 1M triethylammonium bicarbonate (pH 7.5) is added to quench reaction. The resulting 2′,3′-O-bis-substituted N⁶-phenoxyacetyl derivatives of ATP are isolated and purified by a combination of anion-exchange and reverse-phase chromatography to obtain 98-99% pure 2′,3′-substituted N⁶-phenoxyacetyl derivatives ATP as sodium salt.

### Example 15

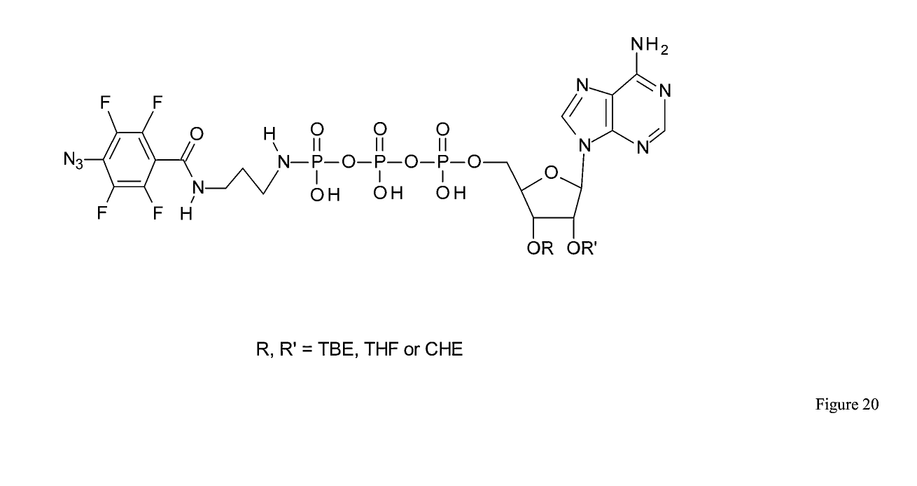

### Synthesis of 2′,3′-O-bis-substituted adenosine 5′-gamma-{1-[3-(4-azido-2,3,5,6-tetrafluorobenzoyl)aminopropyl]amido}-triphosphate (FIG. 20)

Approximately 5 μL, of aqueous solution of 2′,3′-bis-substituted ATP (sodium salt; 0.5M) is mixed with 110 μL, of DMSO solution of α,α-dipyridyldisulfide (0.55M) and triphenylphosphine (0.55M). The mixture is stirred for 15 minutes at room temperature and the reaction product is precipitated with 1 mL of diethyl ether. The mixture is centrifuged, the precipitate is washed with 1 mL of diethyl ether and 1 mL of 0.05M aqueous solution of 3-[(4-azido-2,3,5,6-tetrafluorobenzoyl)amino]propylamine is added. The mixture is kept in dark for 70 minutes at room temperature and the product is precipitated with 10 mL of 6% LiClO₄ in acetone. The precipitate is collected by centrifugation, washed with 1 mL of acetone, 1 mL of diethyl ether and dried under vacuum. The 2′,3′-O-bis-substituted adenosine 5′-gamma-{1-[3-(4-azido-2,3,5,6-tetrafluorobenzoyl)aminopropyl]amido}-triphosphate is isolated and purified with reverse phase HPLC as sodium salt.

### Example 16

### Synthesis of P¹-5′-nicotinamide riboside-P²-5′-[2′,3′-O-bis-substituted]-N⁶-phenoxyacetyl-adenosine pyrophosphate (FIG. 19)

### Synthesis of 5′-imidazolylphosphoryl-nicotinamide riboside

Nicotinamide riboside 5′-phosphate (0.33 mmol) was suspended in dry DMF, 1,1′-carbonyldiimidazole (CDI; 1.6 mmol) and the mixture was stirred for 1 hour at room temperature until the solution turned yellowish in color. Methanol (140 uL) was added and incubated for 30 minutes to quench excess CDI.

### Synthesis of 2,3′-O-bis-substituted-N⁶-phenoxyacetyl-adenosine-5′-monophosphate

2′,3′-O-bis-substituted N⁶-phenoxyacetyl-adenosine (0.66 mmol) is reacted with 2.0 equivalents of POCl₃ in 3.3 mL of trimethylphosphate:lutidine (5:1, v/v) at 0° C. for 30 minutes. Approximately 30 mL of 1M triethylammonium bicarbonate solution (pH 7.5) is added to quench the reaction. The resulting 2′,3′-O-bis-substituted N⁶-phenoxyacetyl derivatives of AMP are isolated and purified by anion-exchange chromatography to obtain 95% pure 2′,3′-bis-substituted N⁶-phenoxyacetyl derivatives AMP as triethylammonium salt.

### Synthesis of P¹-5′-nicotinamide riboside-P²-5′-[2′,3′-O-bis-substituted-N⁶-phenoxyacetyl]-adenosine pyrophosphate

Approximately 1.0 mL of a 0.2M solution of 2′,3′-bis-substituted N⁶-phenoxyacetyl AMP (triethylammonium salt) is added to a solution of 5′-imidazolylphosphoryl-nicotinamide riboside and the mixture is stirred for 3 days at room temperature. The P¹-5′-nicotinamide riboside-P²-5′-{[2′,3′-O-bis-substituted]-N⁶-phenoxyacetyl}-adenosine pyrophosphate is isolated and purified by preparative reverse phase HPLC on a C18 column (47×300 mm) using a gradient of acetonitrile in 100 mM TEAB, pH 8.0. Fractions are pooled, dried on rotary evaporator and co-evaporated with methanol to give 95% pure P¹-5′-nicotinamide riboside-P²-5′-[2′,3′-O-bis-substituted-N⁶-phenoxyacetyl]-adenosine pyrophosphate. Typical yield is 30-50%.

### Example 17

### Synthesis of P¹-5%[2′,3%0-bis-substituted-N⁶-phenoxyacetyl]-adenosine-P¹-(4-oxotetradecyl)-P²-5′-nicotinamide riboside-pyrophosphate

### Synthesis of 4-oxotetradecyl-phosphate

4-oxotetradecyl alcohol (1.0 mmol) is reacted with 2.0 equivalents of POCl₃ in 3.0 mL of trimethylphosphate at 0° C. for 60 minutes. Approximately 30 mL of 1M triethylammonium bicarbonate (pH 7.5) is added to quench the reaction. The resulting 4-oxotetradecyl-phosphate is isolated and purified by anion-exchange chromatography using a conductivity detector to obtain 95% pure 4-oxotetradecyl phosphate as triethylammonium salt.

### Synthesis of nicotinamide riboside 5′-(4-oxotetradecyl)-phosphate

A mixture of 1.5 mmol 4-oxotetradecyl phosphate and 3 mmol of 2,4,6-mesitylene sulfonyl chloride in 5 ml of absolute pyridine is incubated for 2 hours at room temperature. The mixture is added to 1 mmol of nicotinamide riboside (Yang, T. et al. J. Med. Chem., 2007, 50 (26), pp 6458-6461) and incubated for 40 minutes. The reaction is quenched with 20 mL of 1M TEAB (pH 7.5) for 5 hours and then evaporated to a solid residue. The product is isolated by anion exchange chromatography and re-purified by reverse phase chromatography to obtain 95% pure nicotinamide riboside 5′-(4-oxotetradecyl)-phosphate as triethylammonium salt.

### Synthesis of P¹-5′-[2,3′-O-bis-substituted-N⁶-phenoxyacetyl]-adenosine-P¹-(4-oxotetradecyl)-P²-5′-nicotinamide riboside-pyrophosphate

A mixture of 0.5 mmol of 2′,3′-O-bis-substituted-N⁶-phenoxyacetyl-adenosine-5′-monophosphate and 0.75 mmol of 2,4,6-mesitylene sulfonyl chloride in 2.5 mL of DMF:pyridine (4:1, v/v) is incubated for 1 hour. Approximately 0.5 mmol of nicotinamide riboside 5′-(4-oxotetradecyl)-phosphate in 2.5 mL of DMF is added and after 5 minutes the mixture is poured into 100 mL of diethyl ether. Precipitate is washed with diethyl ether (2×50 mL) and dried under vacuum. The P¹-5′-(2′,3′-O-bis-substituted-N⁶-phenoxyacetyl]adenosine-P¹-(4-oxotetradecyl)-P²-5′-nicotinamide riboside-pyrophosphate is purified by reverse phase HPLC. Product yield is approximately 30%.

### Example 18

### UV-Melting Temperature Experiments on Complexes of OXT-Substituted and Unsubstituted Acceptor Probes with Complementary DNA Target Sequence

All melting temperature experiments were performed using a Beckman DU 800 Spectrophotometer (Beckman Coulter, Brea, Calif.). Samples contained a 2 μM concentration of oligonucleotides in buffer containing NaCl (137 mM), KCl (2.7 mM), Na₂HPO₄ (10 mM), KH₂PO₄ (2 mM) at pH 7.4. Temperature was increased from 20° C. to 95° C. at a rate of 1.5° C./minute. Samples were in a 340 μL UV-cell and melting curves were monitored at 260 nm.

The data presented in Table 7 demonstrates that OXT-substitution on acceptor probes impairs hybridization to a complementary target. This is reflected in the reduction of Tm values for OXT-substituted acceptor probes. In addition, two OXT substitutions on a single acceptor probe have a stronger effect on reducing Tm values compared to a single OXT substitution.

TABLE 7
Influence of OXT substitution group on stability of complementary
complexes of acceptor probe and target oligonucleotide

| Oligo | | Tm, | ΔTm, ° C. |
|---|---|---|---|
| (5′→3′) | All are 2′-deoxyribonucleotides | ° C. | (PDE - PTE) |
| Target | AGC AAT AGT TGT GTG GAC CAT AGT A | | |
| sequence | | | |
| Acceptor | | | |
| probes | | | |
| Unsubstituted | TAC TAT GGT CCA CAC AAC TAT TGC T | 68.9 | N/A |
| Single OXT1 | TAC TAT GGT CCA CAC AAC TAT TGC (OXT)T | 67.5 | −1.4 |
| Single OXT2 | TAC TAT GGT CCA CAC AAC TAT TG(OXT)C T | 66.6 | −2.3 |
| Double OXT12 | TAC TAT GGT CCA CAC AAC TAT TG(OXT)C (OXT)T | 66.2 | −2.6 |
| Double OXT23 | TAC TAT GGT CCA CAC AAC TAT T(OXT)G(OXT)C T | 65.4 | −3.5 |

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

What is claimed is:

1. A method of ligase mediated nucleic acid replication comprising:
replicating a nucleic acid using at least one substituted ligase component comprising a thermally labile substitution group,
wherein said substituted ligase component is one or more ligase components selected from the group consisting of a substituted ligase cofactor, a substituted donor probe, a substituted adenylated donor intermediate and a substituted acceptor probe.

2. A method of ligase mediated nucleic acid replication for determining the presence or absence or identity of nucleic acid residues in specified positions of a target nucleic acid comprising:
incubating said target nucleic acid in a reaction mixture comprising a nucleic acid ligase and an acceptor probe; and
a ligase cofactor and a donor probe, or
an adenylated donor intermediate;
wherein at least one of said ligase cofactor, said donor probe, said adenylated donor intermediate or said acceptor probe is a substituted ligase component comprising a thermally labile substitution group; and
monitoring ligation,
wherein the amount of ligation is indicative of the presence or identity of said nucleic acid residue in said specified position of said target nucleic acid or
wherein the absence of ligation is indicative of the absence of said nucleic acid residue in said specified position of said target nucleic acid.

3. The method according to claim 2, wherein said nucleic acid residue in said specified position of said target nucleic acid is a single nucleotide polymorphism (SNP) site.

4. The method according to claim 1, wherein said substituted ligase component prevents or inhibits ligation prior to an initial heat denaturation step.

5. The method according to claim 1, wherein the presence of said substituted ligase component reduces or eliminates formation of off-target ligation products as compared with the corresponding natural or unsubstituted ligase component.

6. The method according to claim 1, wherein said thermally labile substitution group dissociates or cleaves during an initial heat denaturation step.

7. The method according to claim 2, wherein the ligase mediated replication reaction comprises two or more different substituted ligase components, each independently comprising one or more thermally labile substitution groups wherein said thermally labile substitution groups are the same thermally labile substitution group or are different thermally labile substitution groups.

8. The method according to claim 7, wherein at least two of said different thermally labile substitution groups dissociate or cleave to form the corresponding unsubstituted ligase components at different rates.

9. The method according to claim 7, wherein at least two of said different thermally labile substitution groups dissociate to form the corresponding unsubstituted ligase components at different temperatures.

10. The method according to claim 2, wherein said ligase mediated nucleic acid replication is selected from the group consisting of a ligase chain reaction (LCR), an Allele-specific LCR, an Assembly LCR or Ligase Cycling Assembly (LCA), an Asymmetric LCR, a Colony LCR, an Emulsion LCR, a Fast LCR, a Gap Extension Ligation PCR (GEXL-PCR), a Gap Ligation Chain Reaction (Gap LCR), a Hot Start LCR, a Ligation-mediated PCR, a Linear-After-The-Exponential-LCR (LATE-LCR), a Methylation-specific LCR (MSL), a Multiplex Ligation-dependent Probe Amplification, (MLPA), a Multiplex LCR, a Nested LCR, a Quantitative LCR (Q-LCR), a Quantitative real-time LCR (QRT-LCR), a Real-Time LCR, a Reverse Transcription LCR(RT LCR), a Single molecule amplification LCR(SMA LCR), a Touchdown LCR, a nucleic acid ligation, and a ligase mediated DNA sequencing.

11. The method according to claim 2, wherein said ligase mediated replication of nucleic acid is a Hot Start ligase reaction or Hot Start ligase chain reaction.

12. The method according to claim 2, wherein said reaction mixture comprises DNA ligase or RNA ligase and one or more enzymes selected from the group consisting of DNA dependent DNA polymerases, RNA dependent DNA polymerases, DNA dependent RNA polymerases, RNA dependent RNA polymerases, synthetases, nucleases, topoisomerases, transferases, phosphatases, pyrophosphatases, triphosphatases, kinases, glycosylases and restrictases.

13. The method according to claim 1, wherein said nucleic acid is DNA, RNA, LNA, PNA, HNA or a combination thereof.

14. The method according to claim 1, wherein said substituted ligase component further comprises one or more detectable labels.

15. The method according to claim 1, wherein said substituted ligase cofactor is an inactive cofactor and does not support or impedes the transfer of a substituted or unsubstituted adenylate moiety, or equivalent thereof, to said nucleic acid ligase that impedes ligation.

16. The method according to claim 1, wherein said substituted ligase cofactor is an enzyme inactivating cofactor able to transfer a substituted adenylate moiety, or equivalent thereof, to said nucleic acid ligase forming an inactive substituted adenylate-enzyme intermediate that impedes ligation.

17. The method according to claim 16, wherein said enzyme inactivating cofactor is substituted ATP or substituted NAD+.

18. The method according to claim 1, wherein said substituted ligase cofactor is a donor inactivating cofactor that supports a two step transfer of a substituted adenylate moiety, or equivalent thereof, to a 5′-phosphate of donor probe forming an inactive substituted adenylated donor probe that impedes ligation.

19. The method according to claim 18, wherein said donor inactivating cofactor is a substituted ATP or substituted NAD+ comprising one or more thermally labile substitution groups.

20. The method according to claim 19, wherein said substituted ATP cofactor comprises one or more thermally labile substitution groups on its sugar and/or adenine base and/or 5′-triphosphate chain.

21. The method according to claim 19, wherein said substituted ATP has the structure of Formula I: 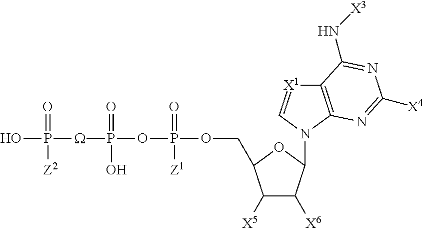
wherein:
X¹ is selected from the group consisting of C—X² and N;
X² is selected from the group consisting of hydrogen, and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
Z¹ is selected from group consisting of OH, OR¹, SH, SR¹, CH₃, CH₂CH₃, Phenyl, BH₃⁻, NH₂, NHR¹, and NR¹R³;
Z² is selected from the group consisting of OH, OR¹, SH, SR¹, NHR¹, NR¹R², F, phosphate, substituted phosphate, substituted polyphosphate, substituted phosphonate, sulfate, sulphonate, O-acyl, S-acyl, NH-acyl, and NR¹-acyl,
wherein Z² is optionally a thermally labile substitution group;
Ω is selected from the group consisting of O, CR¹R², NR¹, and N—OR³;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl,
wherein X³ is optionally a thermally labile substitution group;
X⁴ is selected from the group consisting of hydrogen, NH₂, NHR¹, OH, OR¹, SH, SR¹ and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
X⁵ and X⁶ are each independently selected from the group consisting of hydrogen, OH, 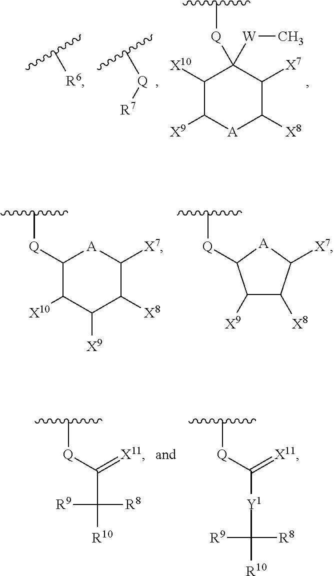
wherein X⁵ and X⁶ are each optionally a thermally labile substitution group;
Q is selected from group consisting O, S, NH, NR¹, NOR¹, CHR¹, and CR¹R²;
R⁶ is selected from the group consisting of inorganic acid residue, or derivative thereof, with the exception of carbonic acid, where the derivatives may include but are not limited to halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R³, R⁷, R⁸, R⁹ and R¹⁰ is independently selected from the group consisting of hydrogen, and a straight or branched optionally substituted hydrocarbyl group having from 1-20 carbon atoms, preferably 1-10 carbon atoms, preferably 1-6 carbon atoms, wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, heterocycle, hydrocarbyl, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting O, S, NH, NR¹, NOR¹, CHR¹, and CR¹R²; and
each A, Y¹ and W is independently selected from the group consisting of O, S, NH, NR¹, NOR¹, CHR¹, and CR¹R²;
wherein at least one of Z², X³, X⁵ and X⁶ are each independently a thermally labile substitution group.

22. The method according to claim 19, wherein the substituted NAD+ has the structure of Formula II: 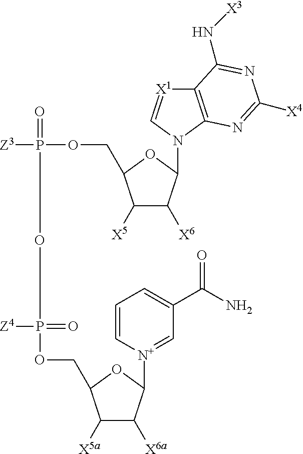
wherein:
X¹ is selected from the group consisting of C—X² and N;
X² is selected from the group consisting of hydrogen, and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
Z³ and Z⁴ are each independently selected from the group consisting of OH, OR¹, SH, SR¹, NHR¹, NR¹R², F, phosphate, substituted phosphate, substituted polyphosphate, substituted phosphonate, sulfate, sulphonate, O-acyl, S-acyl, NH-acyl, NR¹-acyl, CH₃, and BH₃⁻;
wherein Z³ and Z⁴ are each optionally a thermolabile substitution group;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl,
wherein X³ is optionally a thermally labile substitution group;
X⁴ is selected from the group consisting of hydrogen, NH₂, NHR¹, and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁵, X⁶, X^{5a} and X^{6a} is independently selected from the group consisting of hydrogen, OH, 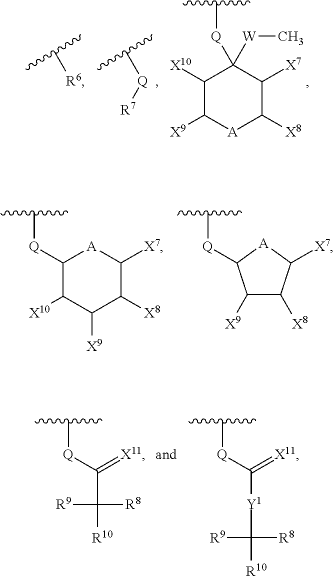
wherein X⁵, X⁶, X^{5a} and X^{6a} are each optionally a thermally labile substitution group;
Q is selected from group consisting O, S, NH, NR¹, NOR¹, CHR¹, and CR¹R²;
R⁶ is selected from the group consisting of inorganic acid residue, or derivative thereof, with the exception of carbonic acid, where the derivatives may include but are not limited to halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R⁷, R⁸, R⁹ and R¹⁰ is independently selected from the group consisting of hydrogen, and a straight or branched optionally substituted hydrocarbyl group having from 1-20 carbon atoms, preferably 1-10 carbon atoms, preferably 1-6 carbon atoms, wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, heterocycle, hydrocarbyl, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting O, S, NH, NR¹, NOR¹, CHR¹, and CR¹R²; and
each A, Y¹ and W is independently selected from the group consisting of O, S, NH, NR¹, NOR¹, CHR¹, and CR¹R²;
wherein at least one of Z³, Z⁴, X³, X⁵, X⁶, X^{5a} or X^{6a} are each independently a thermally labile substitution group.

23. The method according to claim 22, wherein the substituted NAD+ comprises one or more thermally labile groups at the 2′ and/or 3′ positions of adenosine sugar and/or at 2″ and/or 3″ positions of nicotine amide riboside sugar.

24. The method according to claim 19, wherein said thermally labile substitution group is attached to the N⁶ adenine residue of ATP or NAD+ and is selected from the group consisting of methoxycarbonyl, ethoxycarbonyl, 9-fluorenylmethoxycarbonyl, 2,2,2-trichloroethoxycarbonyl, 2-trimethylsylylethoxycarbonyl, tert-butoxycarbonyl, allyloxycarbonyl, benzyloxycarbonyl, phenyloxycarbonyl, p-nitrophenyloxycarbonyl, cyclohexyloxycarbonyl, phenoxyacetyl, methoxyacetyl, benzoyl, acetyl, dimethoxytrityl, monomethoxytrityl, trityl, N,N-dimethylaminomethylidene, N,N-diphenylaminomethylidene, and N,N-dibenzylaminomethylidene.

25. The method according to claim 19, wherein said thermally labile substitution group is attached to the polyphosphate residue of ATP or NAD+ and is selected from group consisting NH-methyl, NH-ethyl, NH-propyl, NH-butyl, NH-phenyl, NH-p-nitrophenyl, NH-o-nitrophenyl, NH-m-nitrophenyl, NH [(4-azido-2,3,5,6-tetrafluorobenzoyl)amino]propyl, imidazolyl, triazolyl, O-2-cyanoethyl, 0-p-nitrophenyl, O-o-nitrophenyl, O-m-nitrophenyl, S-2-cyanoethyl, S-p-nitrophenyl, S-o-nitrophenyl, S-m-nitrophenyl, O-Acetyl, O-benzoyl, O-2,4,6-trimethylcarbonyl, O-phosphoryl, and O-pyrophosphoryl.

26. The method according to claim 1, wherein said substituted donor probe, substituted acceptor probe and/or substituted adenylated donor intermediate have the structures of Formulas III, IV and V, respectively: 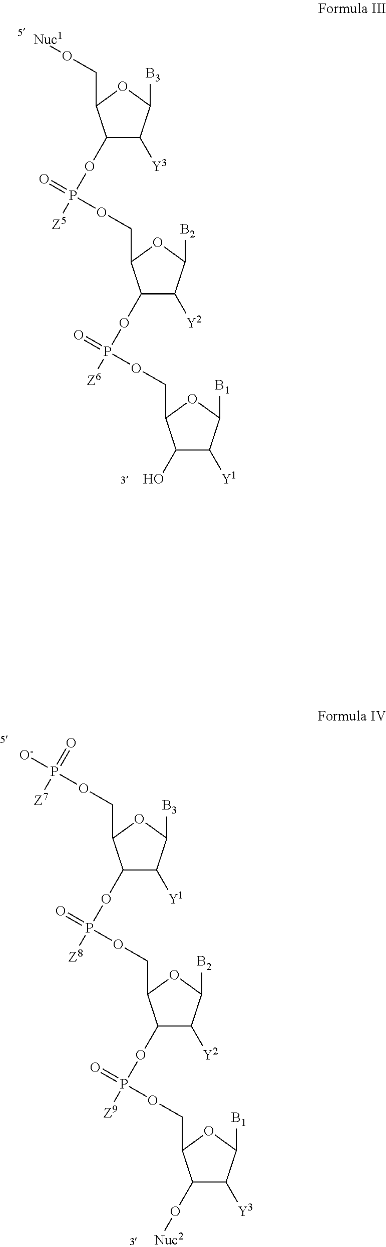 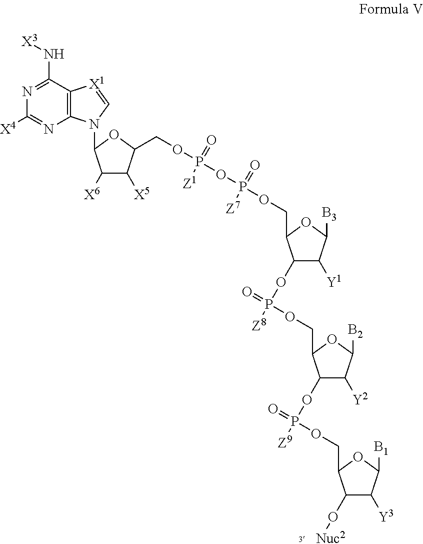
wherein:
B¹, B², and B³ are each independently selected from the group consisting of a substituted or non-substituted purine or pyrimidine, any aza or deaza derivative thereof, and any “universal base” or “degenerate base” of any nucleoside analog
Y¹, Y², and Y³ are each independently selected from the group consisting of H, F, OH and OCH₃; and:
for an acceptor probe (Formula III) Nuc¹ is an oligonucleotide residue within the probe sequence;
for a donor probe (Formula IV) and for adenylate-donor intermediate (Formula V) Nuc² is an oligonucleotide residue within the probe sequence;
at least one of Z¹, Z⁵, Z⁶, Z⁷, Z⁸, Z⁹, X³, X⁵ and X⁶ is independently a thermally labile substitution group;
X¹ is selected from the group consisting of C—X² and N;
X² is selected from the group consisting of hydrogen, and a straight or branched optionally substituted hydrocarbyl group having from 1-20 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl when X¹ is C;
Z¹ and Z⁷ are each independently selected from group consisting of OH, OR¹, SH, SR¹, CH₃, BH₃⁻, NH₂, NHR¹, NR¹R², F, phosphate, substituted phosphate, substituted polyphosphate, substituted phosphonate, sulfate, sulphonate, O-acyl, S-acyl, NH-acyl, and NR¹-acyl;
X³ is selected from the group consisting of hydrogen, acyl, trityl, substituted trityl, alkoxycarbonyl and a straight or branched optionally substituted hydrocarbyl group having from 1-20 carbon atoms, preferably 1-10 carbon atoms, preferably 1-6 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl,
wherein X³ is optionally a thermally labile substitution group;
X⁴ is selected from the group consisting of hydrogen, NH₂, NHR¹, OH, OR¹, SH, SR¹ and a straight or branched optionally substituted hydrocarbyl group having from 1-20 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
X⁵ and X⁶ are each independently selected from the groups consisting of hydrogen, OH, 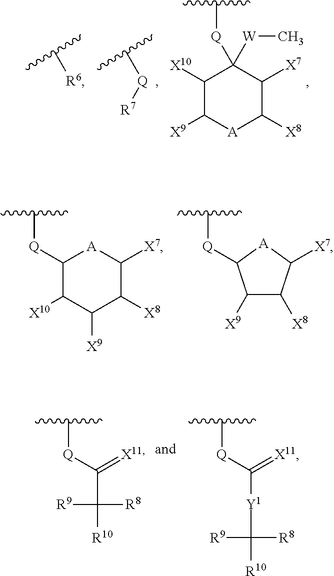
wherein X⁵ and X⁶ are each optionally a thermally labile substitution group;
Q is selected from group consisting of O, S, NH, NR¹, NOR¹, CHR¹, and CR¹R²;
R⁶ is selected from the group consisting of inorganic acid residue, or derivative thereof, with the exception of carbonic acid, where the derivatives are selected from the group consisting of halogen, sulfonate, thio-sulfonate, seleno-sulfate, seleno-sulfonate, sulfate ester, sulfate thioester, sulphite, sulphinate, sulphinic ester, nitrate, nitrite, phosphorus, selenium and boron containing acids;
each R¹, R², R⁷, R⁸, R⁹ and R¹⁰ is independently selected from the group consisting of hydrogen, and a straight or branched optionally substituted hydrocarbyl group having from 1-20 carbon atoms,
wherein the hydrocarbyl is alkyl, alkenyl or alkynyl which may optionally include at least one substituent selected from the group consisting of halo, oxo, thio, hydroxyl, alkoxy, amino, amido, cycloalkyl, heterocycloalkyl, aryl, aryloxy, and heteroaryl;
each X⁷, X⁸, X⁹ and X¹⁰ is independently selected from the group consisting of any substituted or unsubstituted group consisting of acyl, acyloxy, alkenyl, alkenylaryl, alkenylene, alkyl, lower alkyl, alkylene, alkynyl, alkynylaryl, alkoxy, lower alkoxy, alkylaryl, alkylcarbonylamino, alkylsulfinyl, alkylsulfonyl, alkylsulfonylamino, alkylthio, alkynylene, amido, amidino, amino, arylalkynyl, aralkyl, aroyl, arylalkyl, aryl, arylcarbonylamino, arylene, aryloxy, arylsulfonylamino, carbamate, dithiocarbamate, cycloalkenyl, cycloalkyl, cycloalkylene, guanidinyl, halo, halogen, heteroaryl, heteroarylcarbonylamino, heteroaryloxy, heteroarylsulfonylamino, heterocycle, heterocycle, hydrocarbyl, hydrocarbyl, hydrocarbylcarbonyl, hydrocarbyloxycarbonyl, hydrocarbylcarbonyloxy, hydrocarbylene, organosulfinyl, hydroxyl, organosulfinyl, organosulfonyl, sulfinyl, sulfonyl, sulfonylamino, and sulfuryl;
X¹¹ is independently selected from the group consisting O, S, NH, NR¹, NOR¹, CHR¹, and CR¹R²;
each A, Y¹ and W is independently selected from the group consisting of O, S, NH, NR¹, NOR¹, CHR¹, and CR¹R²;
R¹¹ and R¹² are each independently hydrogen or optionally substituted straight or branched hydrocarbyl having from 1-20 carbon atoms, wherein each may independently include at least one substituent selected from halo, oxo, hydroxyl, alkoxy, aryloxy, amino, amido or a detectable label; and
Z⁸ and Z⁹ are each independently OH, SH or a thermally labile substitution group having the structure U-Φ;
wherein U is selected from group consisting of O, S, Se, NR¹¹, and CR¹¹R¹²; and Φ is one or more groups selected from the group consisting of: 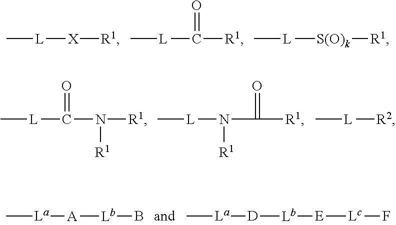
wherein:
L is a straight or branched hydrocarbylene group having between 1-10 carbon atoms;
X is O, S, S(O), S(O)₂, C(O), C(S) or C(O)NH; and
R¹ is hydrogen or a straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl;
k is an integer from 0-2;
R² is an optionally substituted carbocycle, heterocycle, aryl or heteroaryl having between 5-10 atoms;
L^{a}, L^{b} and Le are each independently selected from a bond or a straight or branched hydrocarbylene group having between 1-8 carbon atoms;
A is O, S, S(O), S(O)₂, Se, CR³R⁴, NR³, C(O), C(S) or CNR³;
B is C(O)R³, C(S)R³, C(O)NR³R⁴, OR³ or SR³; and
R³ and R⁴ are each independently hydrogen or straight or branched hydrocarbylene group having from 1-20 carbon atoms, which may optionally include at least one substituent selected from the group consisting of halo, oxo, hydroxyl, alkoxy, amino, amido, cycloalkyl, heterocycloalkyl, aryl, aryloxy, and heteroaryl; and
D is O, S, S(O), S(O)₂, CR⁵R⁶ or NR^{S};
E is O, S, S(O), S(O)₂, CR⁵R⁶ or NR⁶;
F is hydrogen, C(O)R⁷, C(S)R⁷, C(O)NR⁷R⁸, OR⁷ or SR⁷;
R⁵ and R⁶ can each independently be hydrogen, aryl, alkyl, halo, oxo, hydroxyl, alkoxy, aryloxy or amino, or R⁵ and R⁶ can cooperate to form a mono or bicyclic ring consisting 5-10 atoms and including D, R⁵, R⁶, E and L^{b}, provided that when R⁵ and R⁶ cooperate to form a ring; and
R⁷ and R⁸ are each independently selected from the group consisting of aryl, alkyl, halo, oxo, hydroxyl, alkoxy, aryloxy, amino, amido, optionally substituted cycloalkyl, optionally substituted heterocycloalkyl, optionally substituted aryl, optionally substituted aryloxy, and optionally substituted heteroaryl.

27. The method according to claim 26, wherein Φ comprises one or more chemical formulas selected from the group consisting of 4-oxo-1-hexyl, 4-oxo-1-pentyl, 4-oxo-1-tetradecyl, 4-oxo-1-hexadecyl, 4-oxo-1-octadecyl, 4-oxo-1-decadecyl, 5-oxo-1-hexyl, 6-oxo-1-heptyl, 1-methyl-4-oxo-pentyl, 4-methylthio-1-butyl, 5-methyl-4-oxo-hexyl, 1-ethyl-4-oxo-pentyl, 2-phthalimide-1-ethyl, 3-(N-tert-butylcarboxamido)-1propyl, 2-(N-formyl-N-methyl)aminoethyl, and 2-(N-acetyl-N-methyl)aminoethyl.

28. The method according to claim 26, wherein said substituted acceptor probe, substituted donor probe and/or substituted adenylate-donor intermediate impairs hybridization to a complementary target nucleic acid sequence.

29. The method according to claim 2, wherein the presence of said substituted ligase component reduces or eliminates template independent formation of ligation products as compared with the corresponding natural or unsubstituted ligase component.


SEQUENCE LISTING (37 sequences)

SEQ ID NO: 1 - DNA, 18 bp, Artificial Sequence
  synthetic donor-acceptor fragment of ligation complex
gttgcgttagttgtcata

SEQ ID NO: 2 - DNA, 18 bp, Artificial Sequence
  synthetic template fragment of ligation complex
tatgacaactracgcaac

SEQ ID NO: 3 - DNA, 26 bp, Artificial Sequence
  synthetic 4-oxo-tetradecyl (OXT) phosphotriester internucleotide linkage-substituted acceptor probe (OXT-SAP)
ccctgttccagcgtcggtgttgcgtt

SEQ ID NO: 4 - DNA, 26 bp, Artificial Sequence
  synthetic unsubstituted acceptor probe
ccctgttccagcgtcggtgttgcgtt

SEQ ID NO: 5 - DNA, 56 bp, Artificial Sequence
  synthetic matched DNA target (A-T)
cccagactagaggatcaaactatgacaactaacgcaacaccgacgctggaacaggg

SEQ ID NO: 6 - DNA, 56 bp, Artificial Sequence
  synthetic mismatched DNA target (G-T)
cccagactagaggatcaaactatgacaactgacgcaacaccgacgctggaacaggg

SEQ ID NO: 7 - DNA, 51 bp, Artificial Sequence
  synthetic unsubstituted donor probe
agttgtcatagtttgatcctctagtctgggagtattctaggcgactggtac

SEQ ID NO: 8 - DNA, 20 bp, Artificial Sequence
  synthetic 4-oxo-tetradecyl (OXT) phosphotriester internucleotide linkage-modified donor probe (OXT Donor)
ctccactcagtgtgattcca

SEQ ID NO: 9 - DNA, 19 bp, Artificial Sequence
  synthetic 4-oxo-tetradecyl (OXT) phosphotriester internucleotide linkage-modified acceptor probe (OXT Acceptor)
gaaattcttgctcgttgat

SEQ ID NO: 10 - DNA, 20 bp, Artificial Sequence
  synthetic unmodified phosphodiester donor probe (PDE Donor)
ctccactcagtgtgattcca

SEQ ID NO: 11 - DNA, 19 bp, Artificial Sequence
  synthetic unmodified phosphodiester acceptor probe (PDE Acceptor)
gaaattcttgctcgttgat

SEQ ID NO: 12 - DNA, 77 bp, Artificial Sequence
  synthetic G551D mutated CF DNA system sense strand
atatagttcttggagaaggtggaatcacactgagtggagatcaacgagcaagaatttctttagcaaggtgaataact

SEQ ID NO: 13 - DNA, 77 bp, Artificial Sequence
  synthetic G551D mutated CF DNA system antisense strand
agttattcaccttgctaaagaaattcttgctcgttgatctccactcagtgtgattccaccttctccaagaactatat

SEQ ID NO: 14 - DNA, 20 bp, Artificial Sequence
  synthetic unmodified phosphodiester acceptor probe (PDE Acceptor)
tggaatcacactgagtggag

SEQ ID NO: 15 - DNA, 19 bp, Artificial Sequence
  synthetic unmodified phosphodiester donor probe (PDE Donor)
atcaacgagcaagaatttc

SEQ ID NO: 16 - DNA, 20 bp, Artificial Sequence
  synthetic 4-oxo-tetradecyl (OXT) phosphotriester internucleotide linkage-modified acceptor probe (OXT Acceptor)
tggaatcacactgagtggag

SEQ ID NO: 17 - DNA, 18 bp, Artificial Sequence
  synthetic shifted unmodified phosphodiester donor probe (Shifted PDE Donor)
tcaacgagcaagaatttc

SEQ ID NO: 18 - DNA, 21 bp, Artificial Sequence
  synthetic shifted unmodified phosphodiester acceptor probe (Shifted PDE Acceptor)
tggaatcacactgagtggaga

SEQ ID NO: 19 - DNA, 21 bp, Artificial Sequence
  synthetic shifted 4-oxo-tetradecyl (OXT) phosphotriester internucleotide linkage-modified acceptor probe (Shifted OXT Acceptor)
tggaatcacactgagtggaga

SEQ ID NO: 20 - DNA, 19 bp, Artificial Sequence
  synthetic 4-oxo-tetradecyl (OXT) phosphotriester internucleotide linkage-modified donor probe (OXT Donor)
atcaacgagcaagaatttc

SEQ ID NO: 21 - DNA, 18 bp, Artificial Sequence
  synthetic shifted 4-oxo-tetradecyl (OXT) phosphotriester internucleotide linkage-modified donor probe (OXT Donor)
tcaacgagcaagaatttc

SEQ ID NO: 22 - DNA, 19 bp, Artificial Sequence
  synthetic 2 4-oxo-tetradecyl (OXT) phosphotriester internucleotide linkage-modified acceptor probe (2OXT Acceptor)
gaaattcttgctcgttgac

SEQ ID NO: 23 - DNA, 19 bp, Artificial Sequence
  synthetic 4-oxo-tetradecyl (OXT) phosphotriester internucleotide linkage-modified acceptor probe (OXT Acceptor)
gaaattcttgctcgttgac

SEQ ID NO: 24 - DNA, 19 bp, Artificial Sequence
  synthetic unmodified phosphodiester acceptor probe (PDE Acceptor)
gaaattcttgctcgttgac

SEQ ID NO: 25 - DNA, 77 bp, Artificial Sequence
  synthetic wild type CF DNA system sense strand
atatagttcttggagaaggtggaatcacactgagtggaggtcaacgagcaagaatttctttagcaaggtgaataact

SEQ ID NO: 26 - DNA, 77 bp, Artificial Sequence
  synthetic wild type CF DNA system antisense strand
agttattcaccttgctaaagaaattcttgctcgttgacctccactcagtgtgattccaccttctccaagaactatat

SEQ ID NO: 27 - DNA, 19 bp, Artificial Sequence
  synthetic unmodified phosphodiester donor probe (PDE Donor)
gtcaacgagcaagaatttc

SEQ ID NO: 28 - DNA, 20 bp, Artificial Sequence
  synthetic 2 4-oxo-tetradecyl (OXT) phosphotriester internucleotide linkage-modified acceptor probe (2OXT Acceptor)
tggaatcacactgagtggag

SEQ ID NO: 29 - DNA, 21 bp, Artificial Sequence
  synthetic shifted unmodified phosphodiester acceptor probe (Shifted PDE Acceptor)
tggaatcacactgagtggagg

SEQ ID NO: 30 - DNA, 21 bp, Artificial Sequence
  synthetic shifted 4-oxo-tetradecyl (OXT) phosphotriester internucleotide linkage-modified acceptor probe (Shifted OXT Acceptor)
tggaatcacactgagtggagg

SEQ ID NO: 31 - DNA, 19 bp, Artificial Sequence
  synthetic 4-oxo-tetradecyl (OXT) phosphotriester internucleotide linkage-modified donor probe (OXT Donor)
gtcaacgagcaagaatttc

SEQ ID NO: 32 - DNA, 25 bp, Artificial Sequence
  synthetic target oligonucleotide sequence
agcaatagttgtgtggaccatagta

SEQ ID NO: 33 - DNA, 25 bp, Artificial Sequence
  synthetic unsubstituted acceptor probe
tactatggtccacacaactattgct

SEQ ID NO: 34 - DNA, 25 bp, Artificial Sequence
  synthetic 4-oxo-tetradecyl (OXT) phosphotriester internucleotide linkage-modified single OXT1 acceptor probe
tactatggtccacacaactattgct

SEQ ID NO: 35 - DNA, 25 bp, Artificial Sequence
  synthetic 4-oxo-tetradecyl (OXT) phosphotriester internucleotide linkage-modified single OXT2 acceptor probe
tactatggtccacacaactattgct

SEQ ID NO: 36 - DNA, 25 bp, Artificial Sequence
  synthetic 4-oxo-tetradecyl (OXT) phosphotriester internucleotide linkage-modified double OXT12 acceptor probe
tactatggtccacacaactattgct

SEQ ID NO: 37 - DNA, 25 bp, Artificial Sequence
  synthetic 4-oxo-tetradecyl (OXT) phosphotriester internucleotide linkage-modified double OXT23 acceptor probe
tactatggtccacacaactattgct